(19) United States
(12) Patent Application Publication
(10) Pub. No.: US 2023/0374468 A1
(43) Pub. Date: Nov. 23, 2023

(54) FORMULATIONS FOR VIRAL VECTORS

(71) Applicant: LONZA LTD, Visp (CH)

(72) Inventors: Ahmed BESHEER, Basel (CH); Jonas KOST, Basel (CH); Eddy BERTHIER, Basel (CH); Hanns-Christian MAHLER, Loerrach (DE)

(21) Appl. No.: 17/999,775

(22) PCT Filed: May 28, 2021
(86) PCT No.: PCT/US2021/034943

§ 371 Date: Nov. 23, 2022

Related U.S. Application Data
(60) Provisional application No. 63/031,436, filed on May 28, 2020.

(51) Int. Cl. C12N 7/00; C12N 15/86

(52) U.S. Cl. CPC C12N 7/00; C12N 15/86; C12N 2750/14143; C12N 2750/14151

(57) ABSTRACT

Provided are viral vector formulations comprising a globular protein such as an albumin Also provided are viral vector formulations comprising a polysaccharide such as an hyaluronic acid. The provided viral vector formulations have increased stability and reduced viral vector aggregation.

### REFERENCE TO SEQUENCE LISTING SUBMITTED ELECTRONICALLY

The contents of the electronic sequence listing (0250-0002US1_ST25.txt; Size: 1 KB; and Date of Creation Jul. 24, 2023) submitted herewith, is herein incorporated by reference in its entirety.

### FIELD OF THE INVENTION

The present disclosure relates generally to formulations comprising viral vectors. The formulations comprise agents that prevent aggregation of the viral vectors providing stable formulations that are easily administered.

### BACKGROUND

Viral vectors are vehicles for delivery of therapeutic nucleic acids in vaccines and gene therapies. To produce viral vectors, production cells are grown and transfected, typically a plasmid. The viral vector is then harvested from the cells and formulated for use. Achieving high vector concentrations without aggregation is a significant technical bottleneck in manufacturing viral vectors.

Viral particles can form aggregates, which increases their resistance to resist environmental stress and degradation by disinfectants. Aggregation is influenced by cell type and cell-related impurities, the type of virus, biochemical properties of the virus (e.g. virus size and shape, isoelectric point, etc.), physiochemical factors (e.g. pH, ionic strength) as well as operational factors (e.g. process temperature). While research on viral aggregation began in the 1950s, the phenomenon continues to be studied and viewed as one of the main challenges in viral vector manufacturing.

Viral aggregation affects downstream processing of therapeutics and their suitability for administration. Viral aggregation occurs due to electrostatic and hydrophobic interactions and aggregated virus particles can cause complications in downstream processing, leading to significant vector losses and decreased yields during membrane-based processes, such as filtration, and subsequent purification steps. Furthermore, aggregates impact readouts of virus infectivity that provide an indicator of the final product quality.

### BRIEF SUMMARY OF THE INVENTION

The present disclosure is directed to a formulation comprising: a viral vector; a buffer; and a globular protein. In embodiments, the globular protein is albumin, alpha-fetoprotein, vitamin D-binding protein, afamin a globin protein, alpha globulin, beta globulin, gamma globulin, or combinations thereof. In embodiments, the formulation is a liquid. In embodiments, the formulation comprises from about 0.1% to about 5.0% globular protein. In embodiments the formulation comprises from about 0.5% to about 2.0% globular protein. In embodiments, the formulation comprises from about 0.75% to about 1.5% globular protein. In embodiments, the formulation comprises from about 0.8% to about 1.2% globular protein. In embodiments, the formulation comprises about 1.0% globular protein.

The present disclosure is also directed to a formulation comprising: a viral vector; a buffer; and an albumin. In embodiments, the albumin is human serum albumin, bovine serum albumin, ovalbumin or lactalbumin. In embodiments, the formulation is a liquid. In embodiments, the formulation comprises from about 0.1% to about 5.0% albumin. In embodiments, the formulation comprises from about 0.5% to about 2.0% albumin. In embodiments, the formulation comprises from about 0.75% to about 1.5% albumin. In embodiments, the formulation comprises from about 0.8% to about 1.2% albumin. In embodiments, the formulation comprises about 1.0% albumin.

The present invention is also directed to a formulation comprising: a viral vector; a buffer; and a polysaccharide. In embodiments, the polysaccharide is a glycosaminoglycan. In embodiments, the polysaccharide is sodium hyaluronate, heparan, heparan sulfate, heparin, chondroitin, chondroitin sulfate, dermatan, dermatan sulfate, keratan, keratan sulfate, algin, chitosan, chitosan sulfate, dextran, dextran sulfate, or combinations thereof. In embodiments, the polysaccharide is sodium hyaluronate. In embodiments, the formulation is a liquid. In embodiments, the formulation comprises from about 0.01 ng/mL to about 1 mg/mL polysaccharide. In embodiments, the formulation comprises from about 0.05 ng/mL to about 0.5 mg/mL polysaccharide. In embodiments, the formulation comprises from about 0.1 ng/mL to about 0.3 mg/mL polysaccharide. In embodiments, the formulation comprises from about 0.15 ng/mL to about 0.25 mg/mL polysaccharide. In embodiments, the formulation comprises about 0.2 ng/mL polysaccharide. In embodiments, the formulation comprises from about 0.01 ng/mL to about 1 mg/mL sodium hyaluronate. In embodiments, the formulation comprises from about 0.05 ng/mL to about 0.5 mg/mL sodium hyaluronate. In embodiments, the formulation comprises from about 0.1 ng/mL to about 0.3 mg/mL sodium hyaluronate. In embodiments, the formulation comprises from about 0.15 ng/mL to about 0.25 mg/mL sodium hyaluronate. In embodiments, the formulation comprises about 0.2 ng/mL sodium hyaluronate.

In embodiments, the present disclosure also provides formulations comprising both a globular protein and a polysaccharide. In embodiments, the present disclosure provides formulations comprising both albumin and a polysaccharide. In embodiments of these formulations, the polysaccharide is sodium hyaluronate.

In embodiments of the formulations herein, the formulation is a liquid. In embodiments of the liquid formulation, the formulation comprises from about 2 mM to about 100 mM buffer. In embodiments of the liquid formulation, the formulation comprises from about 5 mM to about 50 mM buffer. In embodiments of the liquid formulation, the formulation comprises from about 15 mM to about 25 mM buffer. In embodiments of the liquid formulation, the formulation comprises about 20 mM buffer.

In embodiments of the formulations herein, the buffer is sodium phosphate, L-histidine, tris, succinate, sodium citrate or a combination thereof.

In embodiments, the formulations herein further comprise a sugar. In embodiments of the formulations comprising a sugar, the formulation is a liquid. In embodiments of the liquid formulation comprising a sugar, the formulation comprises from about 50 mM to about 500 mM sugar. In embodiments of the liquid formulation comprising a sugar, the formulation comprises from about 100 mM to about 400 mM sugar. In embodiments of the liquid formulation comprising a sugar, the formulation comprises from about 250 mM to about 350 mM sugar. In embodiments of the liquid formulation comprising a sugar, the formulation comprises about 290 mM sugar. In embodiments of the formulations comprising a sugar, the sugar is sucrose, lactose, glucose, trehalose, or combinations thereof. In embodiments, the sugar is sucrose.

In embodiments, the formulations herein further comprise a surfactant. In embodiments of the formulations comprising a surfactant, the formulation is a liquid. In embodiments of the liquid formulation comprising a surfactant, the formulation comprises from about 0.01% to about 0.1% surfactant. In embodiments of the liquid formulation comprising a surfactant, the formulation comprises from about 0.015% to about 0.025% surfactant. In embodiments of the liquid formulation comprising a surfactant, the formulation comprises about 0.02% surfactant. In embodiments of the formulations comprising a surfactant, the surfactant is polysorbate 80, polysorbate 20 or Kolliphor P188.

In embodiments, the formulations herein further comprise sodium chloride. In embodiments of the formulations comprising sodium chloride, the formulation is a liquid. In embodiments of the liquid formulations comprising sodium chloride, the formulation comprises from about 10 mM to about 500 mM sodium chloride. In embodiments of the liquid formulations comprising sodium chloride, the formulation comprises from about 50 mM to about 300 mM sodium chloride. In embodiments of the liquid formulations comprising sodium chloride, the formulation comprises from about 100 mM to about 200 mM sodium chloride. In embodiments of the liquid formulations comprising sodium chloride, the formulation comprises about 150 mM sodium chloride.

In embodiments of the formulations disclosed herein, the formulation is a freeze-dried solid. In embodiments of the freeze dried formulations, the buffer is sodium phosphate, L-histidine, sodium citrate or a combination thereof In embodiments of the freeze dried formulations, the formulations further comprise a sugar. In embodiments, the sugar is sucrose, lactose, glucose, trehalose, or combinations thereof. In embodiments, the sugar is sucrose. In embodiments of the freeze dried formulations, the formulations further comprise a surfactant. In embodiments, the surfactant is polysorbate 80, polysorbate 20 or Kolliphor P188. In embodiments of the freeze dried formulations, the formulations further comprise sodium chloride.

In embodiments of any of the formulations herein, the formulation has a Z-average of less than or equal to about 50 nm. In embodiments, the formulation has a Z-average of less than or equal to about 40 nm. In embodiments, the formulation has a Z-average of less than or equal to about 31 nm. In embodiments, the formulation has a Z-average of less than or equal to about 25 nm. In embodiments, the formulation has a Z-average of less than or equal to about 20 nm.

In embodiments of any of the formulations herein, the formulation has a polydispersity index of less than or equal to about 0.5. In embodiments, the formulation has a polydispersity index of less than or equal to about 0.35. In embodiments, the formulation has a polydispersity index of less than or equal to about 0.3.

In embodiments of any of the formulations herein, the viral vector is an adeno-associated viral vector, an adenoviral vector, a lentiviral vector, a retroviral vector, a herpes simplex viral vector or a hybrid vector. In embodiments, the viral vector is an adenoviral vector. In embodiments, the viral vector is an adeno-associated viral vector.

In embodiments of any of the formulations herein, the formulation is formulated for administration to a human.

The present disclosure also provides a method for reducing aggregation of a viral vector in a formulation, comprising formulating the viral vector in any of the formulations disclosed herein.

In embodiments, the present disclosure also provides a formulation comprising: from about 10×10⁸ vg/mL to about 10×10¹³ vg/mL of a viral vector; from about 5 mM to about 40 mM sodium phosphate; from about and 200 mM to about 400 mM sucrose; and from about 0.1% to about 5.0% of an albumin. In embodiments, the albumin is human serum albumin, bovine serum albumin or combinations thereof. In embodiments, the formulation comprises from about 10×10⁹ vg/mL to about 10×10¹¹ vg/mL of the viral vector. In embodiments, the viral vector is an adeno-associated viral vector, an adenoviral vector, a lentiviral vector, a retroviral vector, a herpes simplex viral vector or a hybrid vector.

In embodiments, the present disclosure also provides a formulation comprising: from about 10×10⁸ vg/mL to about 10×10¹³ vg/mL of a viral vector; from about 5 mM to about 40 mM sodium phosphate; from about and 200 mM to about 400 mM sucrose; and from about 0.05 mg/mL to about 0.4 mg/mL sodium hyaluronate. In embodiments, the formulation comprises from about 10×10⁹ vg/mL to about 10×10¹¹ vg/mL of the viral vector. In embodiments, the viral vector is an adeno-associated viral vector, an adenoviral vector, a lentiviral vector, a retroviral vector, a herpes simplex viral vector or a hybrid vector.

### BRIEF DESCRIPTION OF DRAWINGS

The following drawings form part of the present specification and are included to further demonstrate exemplary embodiments of certain aspects of the present invention.

FIG. 1 shows a bar plot of viral genome titers for the impact of DNase I digestion and thermal capsid opening step on the AAV2 titration with qPCR as described in Example 5.1.

FIG. 2 shows a bar plot of AAV2 titer quantification of mixed samples compared to not mixed samples upon 24 h storage in Eppendorf tubes as described in Example 5.1.

FIGS. 3A and 3B show transmission electron microscopy (TEM) images of AAV2 samples as described in Example 5.1: (A) AAV2 sample with a titer of 1·10¹⁰ vg/mL prepared with the standard sample preparation protocol and (B) AAV2 sample with a titer of 1·10¹¹ vg/mL prepared with the modified sample preparation protocol.

FIGS. 4A-F show plots of results obtained with dynamic light scattering (DLS) and multiple angle dynamic light scattering (MADLS) as described in Example 5.1: (A) Particle size distribution with back-scatter of four replicates with a titer of 1.0·10¹¹ vg/mL, (B) Correlation curve of back-scatter measurements, (C) Back-scatter, (D) side-scatter, (E) Forward-scatter and (F) MADLS.

FIGS. 5A and 5B are plots of the zeta potential measurement described in Example 5.1: (A) Phase plot and (B) Zeta potential measurement of an AAV2 formulation with a titer of 1·10¹¹vg/mL.

FIG. 6 is a plot of the U2OS-HTB-96 cell viability assay with alamarBlue reagent described in Example 5.1.

FIG. 7 is a bar plot of the fluorescence of U2OS cells after infection with a MOI of 10⁵ and 10⁶ vg/cell of AAV2-CMV-GFP (ex: 488 nm em: 520 nm) as described in Example 5.1.

FIG. 8 is a bar plot of the fluorescence intensity of U2OS-HTB-96 cells after 24, 48, 72, 96 h and 7 days when transduced with an AAV2 MOI of 10³, 10⁴, 10⁵, 10⁶ vg/cell (ex: 460 nm em: 515 nm) as described in Example 5.1.

FIG. 9 is a bar plot of AAV2-CMV-GFP titration results measured with qPCR of formulation study 1 at T0 as described in Example 5.2.

FIG. 10 is a bar plot of fluorescence detected from a GFP expression assay of Formulation study 1 at T0 as described in Example 5.2. The fluorescence of U2OS cells upon transduction with AAV2-CMV-GFP formulations is measured after 24, 48 and 72 h.

FIG. 11 shows an image of the Lyophilization cakes of formulation 3, 4, 6 and 7 (Left to Right) as described in Example 5.3. Formulation 3 and 4 showed smaller cracks in the cake.

FIG. 12 is a bar plot with a summary of all titer quantifications measured with qPCR during Formulation study 2 as described in Example 5.3.

FIG. 13 is a bar plot of the fluorescence in U2OS cells after transduction with different AAV2 formulations stored at 2-8° C. for one or three months compared to T0 and frozen samples stored at −20 and −80° C. for three months as described in Example 5.3.

FIG. 14 is a bar plot of the fluorescence in U2OS cells after transduction with different AAV2 formulations stored at 25° C. for one month compared to T0 and frozen samples stored at −20 and −80° C. for three months as described in Example 5.3.

FIG. 15 is a bar plot of GFP expression in U2OS cells after exposure to freeze-thaw cycles, horizontal agitation stress as well as exposure to 40° C. as described in Example 5.3.

FIGS. 16A-H are plots of particle size distribution with back-scattering of all formulations at T0 as described in Example 5.3: (A) Formulation 1, (B) Formulation 2, (C) Formulation 3, (D) Formulation 4, (E) Formulation 5, (F) Formulation 6, (G) Formulation 7, and (H) Formulation 8.

FIG. 17(A) is a bar plot of the Z-Averages of all formulations during all measured pull points and FIG. 17(B) is a bar plot of the polydispersity indexes of all formulations during all measured pull points as described in Example 5.3.

FIGS. 18A-H are bar plot of sub-visible particles having different sizes as described in Example 5.3: (A) counts of sub-visible particles ≥2 μm in all formulations, (B) counts of sub visible particles ≥2 μm in all placebos, (C) counts of sub-visible particles ≥5 μm in all formulations, (D) counts of sub visible particles ≥5 μm in all placebos, (E) counts of sub-visible particles ≥10 μm in all formulations, (F) counts of sub visible particles ≥10 μm in all placebos, (G) counts of sub-visible particles ≥25 μm in all formulations, (H) counts of sub visible particles ≥25 μm in all placebos.

FIG. 19 is a bar plot of a summary of all zeta potentials measured in Formulation study 2 as described in Example 5.3.

FIGS. 20A and 20B are bar plots of summary pH measurements as described in Example 5.3 for: (A) all formulations and (B) placebo.

FIG. 21 is a bar plot of viral titer showing the impact of 150 mM sodium chloride on AAV2 titer recovery after sterile filtration with different 0.2 μm sterile PVDF filters as described in Example 5.4.

FIGS. 22A-C are plots of particle size distributions measured by DLS of an AAV2 formulation containing no sodium chloride compared to a formulation provided with 150 mM NaCl as described in Example 5.4. (A) formulation without any NaCl, (B) formulation supplemented with 150 mM NaCl, (C) formulation with 150 mM NaCl and sterile filtration through a 0.2 μm Millex PVDF sterile filter.

FIG. 23 is a plot showing the thermal Shift of all samples supplemented with different dyes and different concentrations as described in Example 5.4.

FIGS. 24A and 24B are plots of the (A) fluorescence intensities of AAV2 samples supplemented with 1×, 5× and 10× SYPRO-Orange (B) fluorescence intensities of AAV2 samples supplemented with 1×, 5× and 10× SYBR-Gold as described in Example 5.4.

FIG. 25 shows TEM images used for quantification of empty and full AAV2 capsids as described in Example 5.1. 9 images were taken with 11 kV, one was taken with 20 kV and one was taken with 37 kV.

FIGS. 26A-H show plots of the particle size distribution with back-scattering of all placebos at TO as described in Example 5.1: (A) placebo 1, (B) placebo 2, (C) placebo 3, (D) placebo 4, (E) placebo 5, (F) placebo 6, (G) placebo 7, and (H) placebo 8.

FIGS. 27A-H show plots of the formulations with back-scattering after one month at 25° C. as described in Example 5.1: (A) formulation 1, (B) formulation 2, (C) formulation 3, (D) formulation 4, (E) formulation 5, (F) formulation 6, (G) formulation 7, and (H) formulation 8.

### DETAILED DESCRIPTION OF THE INVENTION

Described herein are viral vector formulations having improved stability and reduced aggregation of viral vectors. The increased stability and reduced aggregation makes the formulations easier to store, transport and administer. Also described herein are methods for increasing stability and reducing viral vector aggregation of a viral vector formulation.

As used herein, “a” or “an” may mean one or more. As used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the method/device being employed to determine the value, or the variation that exists among the study subjects. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% or higher variability, depending on the situation. In embodiments, one of skill in the art will understand the level of variability indicated by the term “about,” due to the context in which it is used herein. It should also be understood that use of the term “about” also includes the specifically recited value.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited elements or method steps.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

### Globular Protein Formulations

The disclosure provides viral vector formulations comprising a globular protein having improved stability and reduced vector aggregation. In embodiments, the disclosure provides a formulation comprising: a viral vector; a buffer; and a globular protein.

In embodiments, the globular protein can be any globular protein suitable to reduce viral aggregation in a viral vector formulation. In embodiments, the formulation comprises more than one type of globular protein. The term globular protein refers to water soluble proteins which are relatively spherical in shape. In some embodiments, the term globular protein refers to a protein with a substantial number of α-helices and/or β-sheets, e.g., 2, 3, 4, 5, 6, 7 or 8 α-helices and/or 2, 3, 4, 5, 6, 7 or 8 β-sheets. In some embodiments, the a-helices and/or β-sheets are folded into a compact structure. In some embodiments, the globular protein comprises hydrophobic amino acid side chains and hydrophilic amino acid side chains, wherein the majority of hydrophobic amino acid side chains are buried in the interior of the globular protein (i.e., not capable of interacting with water) and wherein the majority of hydrophilic amino acid side chains lie on the surface of the globular protein (i.e., capable of interacting with water). In some embodiments, the globular protein comprises hydrophobic amino acid side chains, wherein 60%, 70%, 80% or 90% of the hydrophobic amino acid side chains are buried in the interior of the globular protein. In some embodiments, the globular protein comprises hydrophilic amino acid side chains, wherein 60%, 70%, 80% or 90% hydrophilic amino acid side chains are buried in the interior of the globular protein. In some embodiments, the globular protein has 1, 2, 3 or more disulfide bonds. In some embodiments, the globular protein comprises more than one polypeptide, e.g., the globular protein comprises 2, 3, or 4 polypeptides which firm a three-dimensional structure. In embodiments, the globular protein has not been substantially denatured, i.e., the globular protein substantially retains its three-dimensional structure. Thus, in some embodiments, the disclosure provides a formulation comprising: a viral vector; a buffer; and a globular protein wherein the formulation is suitable for maintaining the globular protein in its native state, i.e., the globular protein is not substantially denatured.

In some embodiments, the globular protein has a molecular weight of 10 kDa to 1200 kDa, about 20 kDa to about 1200 kDa, about 30 kDa to about 1000 kDa, about 30 kDa to about 700 kDa, about 40 kDa to about 500 kDa, about 50 kDa to about 250 kDa, about 50 kDa to about 200 kDa, about 50 kDa to about 150 kDa, or about 50 kDa to about 100 kDa. In some embodiments, the globular protein has a molecular weight of 700 kDa to 1200 kDa, 800 kDa to 1200 kD, 900 kDa to 1200 kDa, or 1000 kDa to 1200 kDa.

In some embodiments, the globular protein has a solubility of about 10 mg/mL to about 150 mg/mL, about 20 mg/mL to about 120 mg/mL, about 30 mg/mL to about 100 mg/mL, about 40 mg/mL to about 90 mg/mL, about 40 mg/mL to about 80 mg/mL, or about 40 mg/mL to about 70 mg/mL.

In embodiments, the globular protein is albumin, alpha-fetoprotein, vitamin D-binding protein, afamin, a globin protein, alpha globulin, beta globulin, gamma globulin or combinations thereof. In embodiments, the globular protein is a water soluble globular protein.

In embodiments, the globular protein is an albumin. In embodiments, the globular protein is a serum albumin. In embodiments, the albumin is human serum albumin, bovine serum albumin, ovalbumin or lactalbumin. In embodiments, the albumin is human serum albumin, bovine serum albumin or combinations thereof. In embodiments, the albumin is human serum albumin. In embodiments, the albumin is bovine serum albumin.

In embodiments, the formulation comprises from about 0.1% to about 5.0% globular protein. In embodiments, the formulation comprises from about 0.5% to about 2.0% globular protein. In embodiments, the formulation comprises from about 0.75% to about 1.5% globular protein. In embodiments, the formulation comprises from about 0.8% to about 1.2% globular protein. In embodiments, the formulation comprises about 1.0% globular protein. In embodiments, the formulation comprises about 0.5%, 1.0%, 1.5%, 2.0%, 2.5%, 3.0%, 3.5%, 4.0%, 4.5% or 5.0% globular protein.

In embodiments, the formulation is a liquid or a gel. In embodiments, the formulation is a liquid. In embodiments, the formulation is lyophilized from a liquid or a gel. In embodiments, the formulation is reconstituted from a lyophilized form.

In embodiments, the disclosure provides a formulation comprising: a viral vector; a buffer; and an albumin.

In embodiments, the formulation comprises more than one type of albumin. In embodiments, the albumin is human serum albumin, bovine serum albumin, ovalbumin or lactalbumin. In embodiments, the albumin is human serum albumin, bovine serum albumin or combinations thereof. In embodiments, the albumin is human serum albumin. In embodiments, the albumin is bovine serum albumin.

In embodiments, the formulation comprises from about 0.1% to about 5.0% albumin. In embodiments, the formulation comprises from about 0.5% to about 2.0% albumin. In embodiments, the formulation comprises from about 0.75% to about 1.5% albumin. In embodiments, the formulation comprises from about 0.8% to about 1.2% albumin. In embodiments, the formulation comprises about 1.0% albumin. In embodiments, the formulation comprises about 0.5%, 1.0%, 1.5%, 2.0%, 2.5%, 3.0%, 3.5%, 4.0%, 4.5% or 5.0% albumin.

In embodiments, the formulation comprising albumin is a liquid or a gel. In embodiments, the formulation is a liquid. In embodiments, the formulation is lyophilized from a liquid or a gel. In embodiments, the formulation is reconstituted from a lyophilized form.

### Polysaccharide Formulations

In other embodiments, the disclosure provides a formulation comprising: a viral vector; a buffer; and a polysaccharide.

In embodiments, the polysaccharide can be any polysaccharide suitable to reduce viral aggregation in a viral vector formulation. In embodiments, the formulation comprises more than one polysaccharide. In embodiments, the polysaccharide is sodium hyaluronate, heparan, heparan sulfate, heparin, chondroitin, chondroitin sulfate, dermatan, dermatan sulfate, keratan, keratan sulfate, algin, chitosan, chitosan sulfate, dextran, dextran sulfate or combinations thereof. In embodiments, the polysaccharide is glycosaminoglycan. In embodiments, the polysaccharide is keratan sulfate. In embodiments, the polysaccharide is sodium hyaluronate.

As used herein, the term “sodium hyaluronate” includes both the conjugate base form hyaluronate and the acid form hyaluronic acid.

In embodiments, the formulation comprises from about 0.01 ng/mL to about 1 mg/mL polysaccharide. In embodiments, the formulation comprises from about 0.05 ng/mL to about 0.5 mg/mL polysaccharide. In embodiments, the formulation comprises from about 0.1 ng/mL to about 0.3 mg/mL polysaccharide. In embodiments, the formulation comprises from about 0.15 ng/mL to about 0.25 mg/mL polysaccharide. In embodiments, the formulation comprises about 0.2 ng/mL polysaccharide. In embodiments, the formulation comprises about 0.05 ng/mL, 0.1 ng/mL, 0.15 ng/mL, 0.2 ng/mL, 0.25 ng/mL, 0.3 ng/mL, 0.35 ng/mL, 0.4 ng/mL, 0.45 ng/mL or 0.5 ng/mL polysaccharide.

In embodiments, the formulation comprises from about 0.01 ng/mL to about 1 mg/mL glycosaminoglycan. In embodiments, the formulation comprises from about 0.05 ng/mL to about 0.5 mg/mL glycosaminoglycan. In embodiments, the formulation comprises from about 0.1 ng/mL to about 0.3 mg/mL glycosaminoglycan. In embodiments, the formulation comprises from about 0.15 ng/mL to about 0.25 mg/mL glycosaminoglycan. In embodiments, the formulation comprises about 0.2 ng/mL glycosaminoglycan. In embodiments, the formulation comprises about 0.05 ng/mL, 0.1 ng/mL, 0.15 ng/mL, 0.2 ng/mL, 0.25 ng/mL, 0.3 ng/mL, 0.35 ng/mL, 0.4 ng/mL, 0.45 ng/mL or 0.5 ng/mL glycosaminoglycan.

In embodiments, the formulation comprises from about 0.01 ng/mL to about 1 mg/mL sodium hyaluronate. In embodiments, the formulation comprises from about 0.05 ng/mL to about 0.5 mg/mL sodium hyaluronate. In embodiments, the formulation comprises from about 0.1 ng/mL to about 0.3 mg/mL sodium hyaluronate. In embodiments, the formulation comprises from about 0.15 ng/mL to about 0.25 mg/mL sodium hyaluronate. In embodiments, the formulation comprises about 0.2 ng/mL sodium hyaluronate. In embodiments, the formulation comprises about 0.05 ng/mL, 0.1 ng/mL, 0.15 ng/mL, 0.2 ng/mL, 0.25 ng/mL, 0.3 ng/mL, 0.35 ng/mL, 0.4 ng/mL, 0.45 ng/mL or 0.5 ng/mL sodium hyaluronate.

In embodiments, the formulation is a liquid or a gel. In embodiments, the formulation is a liquid. In embodiments, the formulation is lyophilized (i.e., freeze-dried) from a liquid or a gel. In embodiments, the formulation is reconstituted from a lyophilized form.

### Combination Formulations

In embodiments, the disclosure provides a formulation comprising: a viral vector; a buffer; a globular protein; and a polysaccharide. In embodiments, the formulation comprises both a globular protein at a concentration disclosed above and a polysaccharide at a concentration disclosed above. In embodiments, the formulation comprises both albumin and a polysaccharide. In embodiments, the formulation comprises both albumin at a concentration disclosed above and a polysaccharide at a concentration disclosed above.

In embodiments, the formulation comprises both albumin and a glycosaminoglycan. In embodiments, the formulation comprises both albumin at a concentration disclosed above and a glycosaminoglycan at a concentration disclosed above.

In embodiments of the combination formulation, the polysaccharide is sodium hyaluronate. In embodiments, the formulation comprises both albumin and sodium hyaluronate. In embodiments, the formulation comprises both albumin at a concentration disclosed above and sodium hyaluronate at a concentration disclosed above.

### Liquid and Gel Formulations

In embodiments, the formulation comprises a buffer. In embodiments, the formulation is a liquid and comprises a buffer. In embodiments, the formulation is a gel and comprises a buffer. In embodiments, the formulation comprises from about 2 mM to about 100 mM buffer. In embodiments, the formulation comprises from about 5 mM to about 50 mM buffer. In embodiments, the formulation comprises from about 15 mM to about 25 mM buffer. In embodiments, the formulation comprises about 20 mM buffer. In embodiments, the formulation comprises from about 5 mM to about 75 mM buffer. In embodiments, the formulation comprises from about 10 mM to about 30 mM buffer. In embodiments, the formulation comprises about 5, 10, 15, 20, 25, 30, 35, 40, 45 or 50 mM buffer.

In embodiments, the formulation is a liquid and the formulation comprises from about 2 mM to about 100 mM buffer. In embodiments, the formulation is a liquid and the formulation comprises from about 5 mM to about 50 mM buffer. In embodiments, the formulation is a liquid and the formulation comprises from about 15 mM to about 25 mM buffer. In embodiments, the formulation is a liquid and the formulation comprises about 20 mM buffer. In embodiments, the formulation is a liquid and the formulation comprises from about 5 mM to about 75 mM buffer. In embodiments, the formulation is a liquid and the formulation comprises from about 10 mM to about 30 mM buffer. In embodiments, the formulation is a liquid and the formulation comprises about 5, 10, 15, 20, 25, 30, 35 or 40 mM buffer.

In embodiments, the buffer is a phosphate buffer, a histidine buffer, a citrate buffer, a TRIS buffer, a HEPES buffer, a tricine buffer, a tetraborate buffer, a MPOS buffer, a glycine buffer or an imidazole buffer. In embodiments, the buffer is sodium phosphate, L-histidine, sodium citrate or a combination thereof.

In embodiments, the formulation further comprises a sugar. In embodiments, the formulation is a liquid and further comprises a sugar. In embodiments, the formulation is a gel and further comprises a sugar. In embodiments, the formulation comprises from about 50 mM to about 500 mM sugar. In embodiments, the formulation comprises from about 100 mM to about 400 mM sugar. In embodiments, the formulation comprises from about 250 mM to about 350 mM sugar. In embodiments, the formulation comprises about 290 mM sugar. In embodiments, the formulation comprises from about 50 mM to about 400 mM sugar. In embodiments, the formulation comprises from about 100 mM to about 340 mM sugar. In embodiments, the formulation comprises from about 200 mM to about 340 mM sugar. In embodiments, the formulation comprises from about 240 mM to about 340 mM sugar. In embodiments, the formulation comprises about 210 mM, 220 mM, 230 mM, 240 mM, 250 mM, 260 mM, 270 mM, 280 mM, 290 mM, 300 mM, 310, mM, 320 mM, 330 mM, 340 mM, 350 mM, 360 mM, 370 mM, 380 mM, 390 mM or 400 mM sugar.

In embodiments, the formulation is a liquid and the formulation comprises from about 50 mM to about 500 mM sugar. In embodiments, the formulation is a liquid and the formulation comprises from about 100 mM to about 400 mM sugar. In embodiments, the formulation is a liquid and the formulation comprises from about 250 mM to about 350 mM sugar. In embodiments, the formulation is a liquid and the formulation comprises about 290 mM sugar. In embodiments, the formulation is a liquid and the formulation comprises from about 50 mM to about 400 mM sugar. In embodiments, the formulation is a liquid and the formulation comprises from about 100 mM to about 340 mM sugar. In embodiments, the formulation is a liquid and the formulation comprises from about 200 mM to about 340 mM sugar. In embodiments, the formulation is a liquid and the formulation comprises from about 240 mM to about 340 mM sugar. In embodiments, the formulation is a liquid and the formulation comprises about 210 mM, 220 mM, 230 mM, 240 mM, 250 mM, 260 mM, 270 mM, 280 mM, 290 mM, 300 mM, 310, mM, 320 mM, 330 mM, 340 mM, 350 mM, 360 mM, 370 mM, 380 mM, 390 mM or 400 mM sugar.

In embodiments, the sugar is a monosaccharide, disaccharide or trisaccharide. In embodiments, the sugar is dextrose, fructose, galactose, glucose, lactose, maltose, ribose, mannose, sucrose, trehalose, cellobiose, chitobiose, or combinations thereof. In embodiments, the sugar is sucrose, lactose, glucose, trehalose, or combinations thereof. In embodiments, the sugar is sucrose.

In embodiments, the formulation further comprises a surfactant. In embodiments, the formulation is a liquid and further comprises a surfactant. In embodiments, the formulation is a gel and further comprises a surfactant. In embodiments, the formulation comprises from about 0.01% to about 0.1% surfactant. In embodiments, the formulation comprises from about 0.015% to about 0.025% surfactant. In embodiments, the formulation comprises about 0.02% surfactant. In embodiments, the formulation comprises from about 0.05% to about 0.50% surfactant. In embodiments, the formulation comprises from about 0.05% to about 0.3% surfactant. In embodiments, the formulation comprises from about 0.1% to about 0.3% surfactant. In embodiments, the formulation comprises about 0.01%, 0.05%, 0.1%, 0.15%, 0.2%, 0.25% or 0.3% surfactant.

In embodiments, the formulation is a liquid and the formulation comprises from about 0.01% to about 0.1% surfactant. In embodiments, the formulation is a liquid and the formulation comprises from about 0.015% to about 0.025% surfactant. In embodiments, the formulation is a liquid and the formulation comprises about 0.02% surfactant. In embodiments, the formulation is a liquid and the formulation comprises from about 0.05% to about 0.50% surfactant. In embodiments, the formulation is a liquid and the formulation comprises from about 0.05% to about 0.3% surfactant. In embodiments, the formulation is a liquid and the formulation comprises from about 0.1% to about 0.3% surfactant. In embodiments, the formulation is a liquid and the formulation comprises about 0.01%, 0.05%, 0.1%, 0.15%, 0.2%, 0.25% or 0.3% surfactant.

Various surfactants as known in the art, and can include anionic surfactants, nonionic surfactants, cationic surfactants, or zwitterionic surfactants.

Some commonly encountered surfactants of each type include:

Ionic surfactants: i.) anionic surfactants (typically based on sulfate, sulfonate or carboxynate anions) e.g. α-olefin sulphate, ammonium octyl/decyl ether sulphate, sodium sulfosuccinates, sodium tridecyl ether sulphate, triethanolamine lauryl sulphate, sodium glyocholate, sodium taurocholate, sodium taurodeoxycholate, N-lauroylsarcosine, alkyl sulfate salts, especially alkali metal or earth alkali metal alkyl sulfate salts, like sodium dodecyl sulfate, lithium dodecyl sulfate etc. or ammonium lauryl sulfate; sodium laureth sulfate, alkyl benzene sulfonate, deoxycholic acid alkali or earth alkali salts or deoxycholic acids, phosphoric acid esters- and salts, ii.) cationic surfactants e.g. based on quaternary ammonium cations like cetyltrimethylammonium bromide or other alkyl trimethylammonium salts, alkyl amine salts like stearyl amine acetate or coconut alkyl amine acetate, benzalkonium chlorides and bromides, for example benzethonium chloride or methylbenzethonium chloride, stearylaminepolyglycolether or oleylaminepolyglycolether.

Zwitterionic (amphoteric) surfactants, like dodecylbetain, dodecyldimethylaminoxid, CHAPS, CHAPSO, BigCHAP, EMPIGEN BB (N-Dodecyl-N,N-dimethylglycine), Lauryldimethylamineoxid, zwittergent 3-08, zwittergent 3-10, zwittergent 3-12, zwittergent 3-14, zwittergent 3-16, etc. or non-ionic surfactants, like alkylpoly ethylene oxid, alkyl polyglycoside, including: octyl glycoside and decyl maltoside, e.g. nonidet P10 or nonidet P40 surfactants, MEGA-8, -9 or -10, Triton X 100 and related surfactants or surfactants of the Tween family, like Tween 20, Tween 40, Tween 60, Tween 80, APO-10, APO-12, C₈E₆, Ci₀E₆, Ci₂E₆, C₂E₈, Ci₂E₉, Ci₂E₁₀, Ci₆Ei₂, Ci₆E₂₁, Heptane-1,2,3-triol, lubrol PX, genapol family, n,-Dodecyl-b-D-glucopyranoside, thesit, pluronic family, etc.

In some embodiments, the surfactant is a combination of at least two surfactants. Preferably one surfactant is a cationic surfactant while the at least one further surfactant is a non-ionic surfactant. In some embodiments, the surfactant is a combination of cetyltrimethylammonium bromide as cationic surfactant and polysorbate, e.g. Tween 20 or Tween 80, as non-ionic surfactant. In some embodiments, the surfactant is not cetyltrimethylammonium bromide

In some embodiments, the surfactant is an anionic surfactant. In embodiments, the surfactant is polysorbate 80, polysorbate 20 or Kolliphor P188.

In embodiments, the formulation further comprises sodium chloride. In embodiments, the formulation is a liquid and further comprises sodium chloride. In embodiments, the formulation is a gel and further comprises sodium chloride. In embodiments, the formulation comprises from about 10 mM to about 500 mM sodium chloride. In embodiments, the formulation comprises from about 50 mM to about 300 mM sodium chloride. In embodiments, the formulation comprises from about 100 mM to about 200 mM sodium chloride. In embodiments, the formulation comprises about 150 mM sodium chloride. In embodiments, the formulation comprises from about 50 mM to about 250 mM sodium chloride. In embodiments, the formulation comprises from about 125 mM to about 175 mM sodium chloride. In embodiments, the formulation comprises about 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM or 250 mM sodium chloride.

In embodiments, the formulation is a liquid and the formulation comprises from about 10 mM to about 500 mM sodium chloride. In embodiments, the formulation is a liquid and the formulation comprises from about 50 mM to about 300 mM sodium chloride. In embodiments, the formulation is a liquid and the formulation comprises from about 100 mM to about 200 mM sodium chloride. In embodiments, the formulation is a liquid and the formulation comprises about 150 mM sodium chloride. In embodiments, the formulation is a liquid and the formulation comprises from about 50 mM to about 250 mM sodium chloride. In embodiments, the formulation is a liquid and the formulation comprises from about 125 mM to about 175 mM sodium chloride. In embodiments, the formulation is a liquid and the formulation comprises about 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM or 250 mM sodium chloride.

### Freeze-Dried (Lyophilized) Formulations

In embodiments, the formulation is a freeze-dried (lyophilized) solid. Freeze-dried solids can be made using processes known in the art, including processes described herein.

In embodiments, the freeze-dried solid comprises a buffer. In embodiments, the buffer is sodium phosphate, L-histidine, tris, succinate, sodium citrate or a combination thereof. In embodiments, the freeze-dried formulation further comprises a sugar. In embodiments, the sugar is sucrose, lactose, glucose, trehalose, or combinations thereof. In embodiments, the sugar is sucrose.

In embodiments, the freeze-dried formulation further comprises a surfactant. In embodiments, the surfactant is polysorbate 80, polysorbate 20 or Kolliphor P188.

In embodiments, the freeze-dried formulation further comprises sodium chloride.

In embodiments, the freeze-dried formulation is made by freeze-drying a liquid or gel formulation as described herein. In embodiments, the freeze-dried formulation is reconstituted to form a liquid or gel formulation as described herein. In embodiments, the freeze-dried formulation is reconstituted in water to form a liquid or gel formulation as described herein.

### Formulation Properties

In embodiments, the viral vector is in a virus particle. Thus, in some embodiments the term viral vector can include the protein capsid particles associated with, e.g., encompassing, the viral vector. In some embodiments, the viral vector is in an infectious viral particle, i.e., it is capable of infecting a host organism. For complete clarity, as used herein, in some embodiments the term viral vector can include a virus particle, e.g., an infectious viral particle. Thus, reference to a viral vector in the formulations or methods described herein can include a viral particle, e.g., an infectious viral particle.

In embodiments, the formulation has properties measured using standard techniques in the art for evaluating viral vector formulations. In embodiments, the Z-average is measured for the formulation. The term “Z-average” as used herein is the intensity weighted mean hydrodynamic size of particles is a solution. In embodiments, the presence of larger particles in a viral vector formulation can indicate vector aggregation. Methods for measuring Z-average are described herein. In embodiments, the Z-average is measured using a dynamic light scattering (DLS). In embodiments, the Z-average is measured using multiple angle dynamic light scattering (MADLS). In embodiments, the Z-average is measured using nano-tracking analysis.

In embodiments, the formulation has a Z-average of less than or equal to about 50 nm. In embodiments, the formulation has a Z-average of less than or equal to about 40 nm. In embodiments, the formulation has a Z-average of less than or equal to about 31 nm. In embodiments, the formulation has a Z-average of less than or equal to about 25 nm. In embodiments, the formulation has a Z-average of less than or equal to about 20 nm.

In embodiments, the polydispersity index (PDI) is measured for the formulation. The term “polydispersity index” as used herein is a measure of the heterogeneity of a sample based on size. In embodiments, the presence of heterogenous particles in a viral vector formulation can indicate viral vector aggregation. Methods for measuring polydispersity index are described herein. In embodiments, polydispersity index is measured using dynamic light scattering. In embodiments, polydispersity index is measured using size-exclusion chromatography, e.g., gel permeation chromatography. In embodiments, polydispersity index is measured using mass spectrometry.

In embodiments, the formulation has a polydispersity index of less than or equal to about 0.5. In embodiments, the formulation has a polydispersity index of less than or equal to about 0.35. In embodiments, the formulation has a polydispersity index of less than or equal to about 0.3.

### Viral Vectors

The viral vector in the formulation can be any viral vector known in the art. In embodiments, the formulation comprises more than one type of viral vector. In embodiments, the formulation comprises more than one viral vector of the same type, where each of the viral vectors comprises a different nucleic acid sequence.

In embodiments, the viral vector is an adeno-associated viral (AAV) vector, an adenoviral vector, a lentiviral vector, a retroviral vector, a herpes simplex viral vector or a hybrid vector. In embodiments, the viral vector is an adenoviral vector. In embodiments, the viral vector is an adeno-associated viral vector. In embodiments, the viral vector is an adeno-associated viral vector of the serotype AAV1, AAV2, AAV3, AAV4, AAVS, AAV6, AAV7 or AAV8.

In embodiments, the disclosure provides a formulation comprising from about 10×10⁸ vg/mL (Viral genome per mL) to about 10×10¹³ vg/mL of a viral vector. In embodiments, the formulation comprises from about 10×10⁹ vg/mL to about 10×10¹¹ vg/mL of the viral vector. In embodiments, the formulation comprises from about 10×10⁵ vg/mL to about 10×10¹⁵ vg/mL of the viral vector. In embodiments, the formulation comprises from about 10×10⁶ vg/mL to about 10×10¹⁴ vg/mL of the viral vector. In embodiments, the formulation comprises from about 10×10⁷ vg/mL to about 10×10¹³ vg/mL of the viral vector. In embodiments, the formulation comprises from about 10×10⁸ vg/mL to about 10×10¹² vg/mL of the viral vector. In embodiments, the formulation comprises about 10×10⁵ vg/mL, 10×10⁶ vg/mL, 10×10⁷ vg/mL, 10×10⁸ vg/mL, 10×10⁹ vg/mL, 10×10¹⁰ vg/mL, 10×10¹¹ vg/mL, 10×10¹² vg/mL, 10×10¹³ vg/mL, 10×10¹⁴ vg/mL, or 10×10¹⁵ vg/mL of the viral vector.

### Administration to Subjects

In embodiments, the formulation is formulated to be administered to a mammal. In embodiments, the formulation is formulated for administration to a human. In embodiments, the formulation is formulated to be administered to a companion animal, e.g. a dog or cat. In embodiments, the formulation is formulated to be administered to a farm animal, e.g., cattle, swine, poultry, sheep, goats or horses.

### Methods of Reducing Aggregation of Viral Vectors

In embodiments, the present disclosure provides methods for reducing aggregation of viral vectors in a formulation. In embodiments, the method for reducing aggregation of a viral vector in a formulation, comprising formulating the viral vector in any of the formulations described herein. In embodiments, the method for reducing aggregation of viral vectors is used in a process for manufacturing a therapeutic protein. In embodiments, the method for reducing aggregation of viral vectors is used in a process for manufacturing a therapeutic antibody. In embodiments, the method for reducing aggregation of viral vectors is used in a process for manufacturing a vaccine. In embodiments, the method for reducing aggregation of viral vectors is used in a process for developing a therapeutic protein. In embodiments, the method for reducing aggregation of viral vectors is used in a process for developing a therapeutic antibody. In embodiments, the method for reducing aggregation of viral vectors is used in a process for developing a vaccine.

### Formulations

In embodiments, the present disclosure provides a formulation comprising:
from about 10×10⁸ vg/mL to about 10×10¹³ vg/mL of a viral vector;
from about 5 mM to about 40 mM sodium phosphate;
from about and 200 mM to about 400 mM sucrose; and
from about 0.1% to about 5.0% of an albumin.

In embodiments of the formulation the albumin is human serum albumin, bovine serum albumin or combinations thereof. In embodiments, the formulation comprises from about 10×10⁹ vg/mL to about 10×10¹¹ vg/mL of the viral vector. In embodiments, the viral vector is an adeno-associated viral vector, an adenoviral vector, a lentiviral vector, a retroviral vector, a herpes simplex viral vector or a hybrid vector. In embodiments, the viral vector is an adenoviral vector. In embodiments, the viral vector is an adeno-associated viral vector. In embodiments, the viral vector is an adeno-associated viral vector of the serotype AAV1, AAV2, AAV3, AAV4, AAVS, AAV6, AAV7 or AAV8.

In embodiments, the present disclosure provides a formulation comprising
from about 10×10⁸ vg/mL to about 10×10¹³ vg/mL of a viral vector;
from about 5 mM to about 40 mM sodium phosphate;
from about and 200 mM to about 400 mM sucrose; and
from about 0.05 mg/mL to about 0.4 mg/mL sodium hyaluronate.

In embodiments of the formulation the formulation comprises from about 10×10⁹ vg/mL to about 10×10¹¹ vg/mL of the viral vector. In embodiments, the viral vector is an adeno-associated viral vector, an adenoviral vector, a lentiviral vector, a retroviral vector, a herpes simplex viral vector or a hybrid vector. In embodiments, the viral vector is an adenoviral vector. In embodiments, the viral vector is an adeno-associated viral vector. In embodiments, the viral vector is an adeno-associated viral vector of the serotype AAV1, AAV2, AAV3, AAV4, AAVS, AAV6, AAV7 or AAV8.

### Examples

It will be readily apparent to one of ordinary skill in the relevant arts that other suitable modifications and adaptations to the methods and applications described herein can be made without departing from the scope of any of the embodiments. The below examples are included herewith for purposes of illustration only and are not intended to be limiting.

### 1 Abbreviations Used

AAV Adeno-Associated Virus
P188 Kolliphor P188
PS80 Polysorbate 80
qPCR Quantitative Polymerase Chain Reaction
NTC No Template Control
TEM Transmission Electron Microscopy
DSF Differential Scanning Fluorimetry
DLS Dynamic Light Scattering
MADLS Multi Angle Dynamic Light Scattering
SST System Suitability Test
Pen/Strep Penicilin and Streptomycin
FBS Fetal Bovine Serum
GFP Green Fluorescence Protein
Vg/mL Viral genome per mL
bp Basepair
PDI Polydispersity Index
dsDNA Double-Stranded Desoxyribonucleic Acid
MOI Multiplicity of Infection (Number of viral genomes added to one cell for transduction)
F1 20 mM L-Histidine, 290 mM Sucrose, pH 5.5, 0.02% PS80, 1.095·10¹¹ vg/mL
F2 20 mM L-Histidine, 290 mM Sucrose, pH 6.8, 0.02% PS80, 1.095·10¹¹ vg/mL
F3 20 mM Sodium-Citrate, 290 mM Sucrose, pH 6.8, 0.02% PS80, 1.095·10¹¹ vg/mL
F4 20 mM Sodium phosphate, 290 mM Sucrose, pH 6.8, 0.02% PS80, 1.095·10¹¹ vg/mL
F5 20 mM Sodium phosphate, 290 mM Sucrose, pH 6.8, 0.02% PS80, 1.095·10¹¹ vg/mL
F6 20 mM Sodium phosphate, 290 mM Sucrose, pH 6.8, 1% Human Albumin, 1.095·10¹¹ vg/mL
F7 20 mM Sodium phosphate, 290 mM Sucrose, pH 6.8, 0.2 mg/mL Sodium Hyaluronate, 1.095·10¹¹ vg/mL
F8 20 mM Sodium phosphate, 290 mM Sucrose, pH 6.8, 0.001% P188, 1.095·10¹¹ vg/mL

### Abstract

Background: Adeno-associated virus serotype 2 (AAV2) has emerged as a popular vector in gene therapy. Based on their ability to cause efficient and stable transgene expression without exhibiting genotoxicity, these vectors are promising for therapeutic approaches [1]. Even though an AAV2 based gene therapy has already entered the market, there is little reported literature on their formulation development.

Purpose: Reported here are the evaluation and development of analytical methods used for characterization of AAV2 and the application of these strategies in a three-month formulation stability study to improve and expand the knowledge of AAV2 formulations.

Methods: To characterize AAV2 samples the following methods were used: Inverted Terminal Repeat qPCR, infection of U2OS-HTB-96 cells with AAV2-CMV-GFP, transmission electron microscopy (TEM), Dynamic Light Scattering (DLS), and zeta potential

Results: During method evaluation and development, it was demonstrated that addition of the enzyme DNase I and a thermal capsid-opening step prior to vector quantification using a universal ITR qPCR resulted in improved accuracy. It was determined that 58% of all capsids contain a vector genome using TEM. In terms of transgene expression, the formulation stability study showed that samples stored in a liquid or frozen-state performed better than freeze-dried. Comparing different buffer systems, the transgene expression was significantly higher when the vectors are stored in L-histidine or sodium phosphate compared to sodium citrate buffer. Additionally, it was observed that the expression rapidly decreased in a temperature dependent fashion upon storage. In contrast to the changes in expression, the titers stayed constant over the course of the study and were only impacted by agitation stress. Moreover, it was demonstrated that vector aggregation occurs during compounding. The addition of 1% human albumin prevented aggregation and improved the vector stability profile with regard to expression.

Conclusions: The purpose of this Project was to improve and expand knowledge related to AAV2 formulations. This was achieved by evaluation and development of several analytical methods for characterization of AAV2 and subsequent formulation study. It was shown that transgene expression of AAV2 was impacted by the formulation buffer, pH and storage condition. Parameters like changes in titer were less affected. Nevertheless, a strategy is provided to further improve formulation stability and prevent aggregation by the addition of albumin.

### 2 Introduction

Recently, gene therapies have gained a lot of attention due to their success in clinical trials [2]. Gene therapy promises solutions for yet unmet medical needs such as inherited diseases [3]. The therapy principle is based on the replacement of a defective gene, responsible for the disease or the delivery of a therapeutic gene as a treatment [4]. Several different vectors are used for facilitating the gene transfer. There are viral and non-viral vectors, and the latter can be divided into physical, mechanical, and chemical methods [5]. Physical methods such as laser irradiation, electroporation and the use of a gene gun have improved dramatically but their use is limited to a local administration [6]. Chemical and synthetic methods, such as liposomes and cationic polymers, are among the most successful and widely used non-viral carriers [7].

Retro-, lenti-, herpes-, adenovirus and adeno-associated viruses are representatives of viral vectors used in gene therapy [8]. See Table 1. These vectors can be categorized into two groups. The first group is able to stably integrate their genome into the host cell chromatid. Retro-, lenti- and herpes viruses are member of that group. The second group deposits its viral genome into the extrachromosomal space. Representatives of that group are adenoviruses and adeno-associated viruses [8]. Retroviruses are able to transform their single stranded RNA into a double stranded DNA that stably integrates into the host cell genome [9]. This permanent integration enables a continuous and long-lasting expression. All retroviruses require mitosis to achieve a stable integration [10]. Despite disadvantages such as mutagenicity and the need for mitotically active cells, retroviral gene therapies have registered some success. They are frequently applied in ex vivo transduction of T-cells to express a chimeric T-cell receptor (CAR-T) against cancer cells [11]. Another application is represented by Strimvelis, a marketed ex vivo retroviral therapy against severe inherited combined immunodeficiency [12].

Recently, a decline in the use of retroviruses and a shift to Lentiviruses in clinical trials has been recorded [13]. Lentiviruses belong to the family of retroviruses, but have the special ability of infecting non-dividing cells [14]. A major risk of lentiviruses is the stimulation of an immune response that compromises the safety and efficiency profile [15].

Herpes simplex virus type 1 is under investigation for treating diseases within the central nervous system as well as for the lysis of cancer cells [16].

Adenoviruses represent a member of the second group of viral vectors that deliver their genome extrachromosomally. Similar to the lentiviruses, they can infect non-dividing cells. Adenoviruses contain a double stranded DNA [17]. The advantages of adenoviral vectors are their high transduction and expression efficiency. Their high immunogenicity represent a major disadvantage [18].

However, the development path of such promising viral therapies is challenging. In 1999 the first patient with an ornithine transcarbamylase (OTC) deficiency was treated with a recombinant adenovirus and passed away after experiencing a severe immune response that resulted in multiorgan failure [19]. In another study in 2000, four children with severe combined immunodeficiency developed a leukemia like T-cell proliferation after ex vivo treatment of their CD34+ cells with a retrovirus [20].

With the discovery and development of adeno-associated viruses (AAV), the field of gene therapy experienced a quantum leap. Their ability to infect dividing- and non-dividing cells and cause stable in vivo protein expression without leading to any genotoxicity or a severe immune response makes them the most promising viral vectors at the moment. Many AAV therapies have transitioned to clinical trials and to the market [21].

In 2012, the first AAV-based gene therapy named Glybera was approved to market. It was developed to treat inherited familial lipoprotein lipase deficiency. Clinical studies have shown that this AAV1-based therapy significantly reduces the triglyceride concentration in the blood for up to 14 weeks. This is accompanied with a decreased incidence of pancreatitis and abdominal pain [22]. In 2018 Glybera was withdrawn from the market because of inadequate long-term efficiency and lack of commercial viability [23]. A big breakthrough in gene therapy came with the approval of Luxturna in 2017. It is an AAV2-based gene therapy that delivers the missing gene to produce the retinal pigment epithelium protein [24]. The therapy was developed by Spark Therapeutics to treat retinal dystrophy [13]. Most recently, AveXis received FDA approval for their gene therapy Zolgensma, a one-dose treatment against spinal muscular atrophy. The disease is caused by mutation or deletion of the survival moto neuron 1 gene. Zolgensma utilizes AAV9 vectors to deliver the missing or defective gene to skeletal muscles [25]. Even though its high price generated many negative headlines, the success of this therapy is an important milestone in proofing the concept for other viral gene therapies [26].

TABLE 1
Marketed AAV2 therapies, Glybvera was withdrawn from the market in 2018

| | Scientific | Date of | Approving | | | Virus | |
|---|---|---|---|---|---|---|---|
| Tradename | Name | Approval | Agency | Indication | Manufacturer | (Serotype | Publication |
| Glybera | Alipogene | Nov. 2012 | EMA | Lipoprotein | uniQure | Adeno- | [13] |
| | tiparvovec | | | lipase | | associated | & |
| | | | | deficiency | | virus 2 | [22] |
| | | | | | | (AAV2) | |
| Luxturna | Voretigene | Dec. 2017 | FDA | Retinal | Spark | Adeno- | [13] |
| | neparvovec- | | | dystrophy | Therapeutics | associated | & |
| | rzyl | | | | | virus 2 | [24] |
| | | | | | | (AAV2) | |
| Zolgensma | Onasemnoge | May 2019 | FDA | Spinal | AveXis | Adeno- | [25] |
| | ne | | | muscle | | associated | & |
| | abeparvovec | | | atrophy | | virus 2 | [26] |
| | -xioi | | | | | (AAV9) | |

### 2.1 Adeno-Associated Viruses

Due to their success and promising properties, AAV vectors are currently subjects of viral gene therapy research. Even though knowledge about AAVs has significantly increased over the years, there is little reported literature on formulation development of AAVs. The few contributions made in the field of AAV formulation development as well as their molecular compositions and infectivity pathways will be further elaborated in the following section.

Croyle and colleagues made one of the first contributions to this field. They conducted an AAV and adenovirus stability study and identified several process factors and excipients that influenced the stability of viral vectors. They observed that AAVs are quite stable when freeze-dried. No loss of infectious titer was observed when stored at 25° C. for up to three months. Freezing AAV formulations to −80° C. resulted in a bigger infectious titer loss, than freezing to −20° C. According to them, this loss is associated with a pH drop upon freezing. However, proper selection of excipients and buffer lead to a stable AAV infectious titer when stored at −80° C., −20° C., 2-8° C., 25° C. for over five months [27].

Another group published a second study regarding the appropriate shipping- and storage temperature for recombinant AAVs (rAAV). They investigated the influence of different storage temperatures such as −80° C., −20° C., 2-8° C., 25° C. and 37° C. on the transduction efficiencies in vitro and in vivo. The virus stock, stored at −80° C. remained stable for up to one month and did not record any loss in transduction efficiency, even though the AAVs were stored without any cryoprotectants. With increasing temperature, the transduction efficiency declines. Especially in the first days of storage, higher temperatures such as 37° C. lead to bigger transduction efficiency drops compared to storage temperatures of 25° C. Furthermore, the study showed that despite a rapid decline of transduction efficiency, some portion of the virus stayed infectious even when stored at 37° C. [28].

A more recent study tested different AAV storage temperatures, buffer solutions and compatibilities of different clinical materials. They used vector copy number and transgene expression as markers for the vector stability. An in vivo decrease in transgene expression was only seen after heating the AAVs to 72° C. for 20 min or exposing them 10 min with UV light. Temperatures such as 55° C. for 20 min or four freeze-thaw cycles had no impact on transgene expression. They also exposed AAV1 vectors with human serum containing complement proteins. Even such conditions did not cause any loss in transgene expression, although more than 70% of the population carries AAV1 immunity. In parallel, a compatibility study was performed with frequently used clinical materials and diluents. The influence of stainless steel needles, and polyethylene and glass surfaces on transgene expression were tested. Additionally, they looked into different frequently used diluents for clinical applications such as phosphate buffer, lactate ringer solution and gadolinium. No material nor diluent resulted in decreased transgene expression. Even different pHs ranging between 5.5 and 8.5 had no impact on the expression [29].

All of the above studies only monitored the infectious titer or the transduction efficiency. No additional physico-chemical characterizations were performed.

Wright and colleagues published a study in which they additionally monitored vector aggregation. Symmetric capsid structures and additional ionic interactions between oppositely charged capsid pockets result in low AAV solubility. Aggregation therefore frequently occurs in high titer formulations with titers >10¹⁴ vg/mL. However, aggregation was also observed at much lower titers, when exposed to freeze-thaw cycles. They showed that several excipients, mainly salts, are able to prevent vector aggregation. This effect is caused by ionic strength and is not dependent on a specific ion species. Charged amino acids, for example, were able to prevent aggregation only at the same ionic strengths as certain salts. In addition, the publication showed that purification processes of AAVs have a crucial impact on vector aggregation. A nuclease treatment after purification significantly reduced vector aggregation. This finding suggests that residual host cell proteins and DNA fragments facilitate ionic bonds between capsids. High ionic strength formulations had no negative effect on the transduction efficiency nor the transgene expression [30].

To better understand the properties influencing the stability of AAVs, there is a need to dig deeper into the capsid- structure and characteristics. Adeno-associated viruses are members of the parvovirus family [31]. They have icosahedral-shaped capsids consisting of 60 proteins with a diameter of around 25 nm [32]. AAV capsids have an isoelectric point of around 6.3. The three dimensional structures of most serotypes have been elucidated by cryoelectron microscopy [33]. In case of AAV2, structural proteins named, VP1, VP2, VP3 make up the capsid in a ratio of 1:1:10, respectively. VP3 is not only the most abundant capsid protein it is also 65 amino acids shorter on the N-Terminus than the other structural proteins [34]. VP1 and VP2 seem to be crucial for a successful infection of cells. On their N-Terminus they contain a phospholipase A₂ (PLA₂) and a nuclear localization signal (NLS) domain that is buried within the capsid. When deleting the VP1/2 capsid proteins or mutating its PLA₂ domain, the virus loses its transduction activity, even though the capsid structure stays intact [35]. For successful transduction, the buried PLA₂ and NLS need to be externalized. The externalization is performed through a channel at the fivefold symmetry axis. Since this pore is rather small, it is unlikely that the bulky VP2 N-Terminus is externalized [36]. Exactly 12 pores are located on an AAV2 capsid. Mutation or deletion of pore forming amino acids have shown that no or only a decreased PLA₂ activity and VP1 N-Terminus externalization occurs. Such mutations had no impact on receptor mediated capsid uptake, but strongly affected the virus infectivity [37]. A more recent study has shown that a change in pH can induce a conformational change of the VP1 N-Terminus. A pH drop from 7.5 to 5 will cause a reversible conformational change and externalization of the VP1 N-Terminus, which enables an endosomal escape. No endosomal escape occurred when treating the cell with bafilomycin A1, a H+ ATPase inhibitor that prevents the cell from keeping its endosomal pH low. This illustrates the necessity of a low pH environment for facilitating the endosomal escape [33]. Similar to a pH induced conformational change, a temperature increase can cause a VP1 externalization. Looking at the thermal stability of AAV capsids in detail, it was shown that capsid melting temperatures differ by more than 20° C. between the most stable and least stable AAV serotype. AAVS is the most stable serotype with a melting temperature of 90° C. compared to the least stable AAV2 with a melting temperature of 70° C. Experiments showed that VP1 does not contribute to the thermal stability, neither does the buried surface area nor the primary amino acid sequence. X-ray crystallography of the capsids revealed that the AAV2 serotype is a much more flexible protein complex compared to AAVS, which could explain the lower capsid melting temperature. But a clear explanation for the huge difference in thermal stability is still pending. Nevertheless, these findings add important value to the characterization of capsids [32].

Capsids are not only responsible for the protection of the genome, they are also important for cellular virus uptake. A receptor mediated clathrin endocytosis is responsible for uptake [38]. After two hours, the virus genome is already located in the nucleus [39]. Serotypes bind to different specific receptors, which explains their tissue tropism [21]. Adeno-associated virus 2 mainly uses the heparan sulfate proteoglycan (HSPG) to enter the cell. Four co-receptors, such as the fibroblast growth factor (FGFR), the hepatocyte growth factor (HGFR), laminin receptor and integrin α_{V}β₁/α_{V}β₅, are known to support uptake [40]. Knocking out HSPG resulted in infection resistance for AAV2 [41]. Meanwhile, the knockout of the FGFR1 receptor had no significant impact on the host cell infection [42]. Even though AAV4 only shares 53% sequence homology with AAVS, both require sialic acid to enter the cell [21]. A more recent study shows the importance of a newly discovered receptor, KIAA0319L. KIAA0319L knockout cells were resistant to all AAV infections [42]. However, the serotype specific receptor interactions are responsible for the AAV tissue tropism [4].

AAV stability is not only impacted by the capsid proteins but also of the genome length, osmotic pressure and pH of the environment. AAVs with smaller genome sizes have higher DNA release temperatures. Arising interactions of the vector DNA with Histidine residues of the capsid can be responsible for the stabilizing effect at lower pH. These additional interactions at low pH might protect and stabilize the virus in the endosome [43]. This illustrated that not only the capsid structure contributes to the virus stability, but also the capsid-gene interaction is crucial. It is worthwhile to particularly have a look at the genome structure of an AAV.

A wtAAV is packed with a 4.7 kb single stranded DNA [44]. The genome contains three open reading frames (ORFs) [45]. The Rep ORF encodes for four proteins responsible for the transcriptional regulation and viral replication. Expression of different Rep proteins is controlled by splicing. A spliced RNA encodes for Rep68 and Rep40 while an unspliced RNA encodes for Rep52 and Rep72 [46]. The Cap ORF encodes for the three structural capsid proteins VP1, VP2, VP3 [47]. Assembly-activating protein (AAP) sits on the Cap gene and represents the third ORF, since it is transcribed with a different reading frame [45]. A 145 base pair long T-shaped hairpin forming inverted terminal repeat (ITR) flanks both genes [48]. ITRs are crucial for vector replication, because they enable DNA polymerase binding [49]. Besides these sequences, there are three different promotors located on the DNA. P40 is responsible for inducing the transcription of the Cap gene. P5 and P19 produce the Rep protein transcripts [50]. Compared to wtAAV, the Rep and Cap gene in a recombinant AAV (rAAV) are replaced with a therapeutic transgene and a respective promotor. Whereas the ITR sequences are kept [51].

Most of the delivered AAV genome is stored extrachromosomal in circular form [52]. Up to 99.5% of the vector DNA can be found in episomal form and is not integrated into the host chromosome [53]. A small percentage integrates in a specific locus of the human chromosome 19 [54].

The following section elaborates analytical methods used for diverse AAV characterizations.

### 2.2 Analytical Methods

### 2.2.1 Strength and Dose Determination with Quantitative Polymerase Chain Reaction (qPCR)

The number of viral genomes delivered usually correlates with the therapeutic effect [56]. Titer measurements are therefore key assays for dose assessments [57]. Several methods exist for titer determination including quantitative realtime PCR, Southern blotting and Ultraviolet spectrophotometry [55]. Even though UV-spectrophotometry is a fast and economical approach, its accuracy is a drawback. Host cell impurities, buffers and excipients impair the accuracy of this technique [58].

Quantification of DNA-amplicons using qPCR requires either a SYBR-Green dye that binds to the double stranded DNA and starts to fluorescent, or a labeled TaqMan probe that binds to the ssDNA and starts to fluoresce upon removal by DNA-Polymerase [59]. Despite its high price, the TaqMan approach is the most frequently used quantification approach. Its high sensitivity and specificity makes it a very reliable strategy. And the high reproducibility, and high comparability to TaqMan strategies makes it an attractive approach [62]. SYBR-Green sensitivity could be compromised by the formation of primer-dimers and of secondary structures [60]. Nevertheless, SYBR-Green is frequently used due to its low price and simplicity [61]. SYBR-Green additionally requires a melt-curve step to confirm specificity [63].

Titer quantification with qPCR evolved to be the most frequently used method for viral titer quantification, since it is rapid, easy to process, and has a broad dynamic range [56]. Often qPCR underestimates the viral titer because ITR fragments form hairpin structures [49]. Such structures interfere with the primer annealing and artificially decrease the titer. Treating samples with an endonuclease prior to quantification resolves the problem of underestimation and additionally improves the inter-assay variability. Assays targeting a region for amplification within the center of the transgene are less affected by ITR hairpin structure caused inaccuracies [64].

Most of the developed qPCR strategies target a specific-transgene sequence [65]. However, commonly used promotors such as the CMV-promotor are also promising amplification targets, because they offer a broader application range [66]. Implementations of such promotor-specific or transgene-specific qPCR measurements are limited because they need to be newly developed for vectors carrying a different transgene or promotor.

Aurnhammer and colleagues developed a universal ITR specific qPCR approach that can be applied to all AAV2. The location of the primers and the probe is within the ITR sequence [65]. Despite these advantages, experiments have shown that ITR qPCR measurements constantly overestimate the viral titer and additionally have high inter-laboratory variabilities. Systematic overestimation is created by single-cut linearized plasmid standards. Decreased amplification efficiency of ITRs embedded in a plasmid can be a reason for the overestimation. Plasmid standards that are cut on both ends of the ITR sequence will resolve this overestimation [67]. The disadvantages of qPCR are that commercially available standard material only exists for AAV2 and 8 and that the quantification accuracy is influenced by the secondary structure of the amplicon.

Recently, a new PCR method, named droplet digital PCR (ddPCR) emerged as a well suited alternative for AAV titer determination [68]. As the name indicates, a sample is divided into around 20 000 droplets. These droplets undergo traditional PCR cycles and accordingly get stained and analyzed with flow cytometry. Droplets containing one or more DNA amplicons are classified as positive and empty droplets are classified as negative. With the help of Poisson statistics, the average number of DNA molecules can be calculated without the need of a DNA standard [69]. Results have shown that ddPCR performed better AAV titrations than a traditional qPCR in respect to robustness and assay variability [70].

However, the analytical method, amplification sequence and fluorescent dye not only affect the accuracy, sensitivity and robustness of an AAV titration, but the sample preparation also has an impact. Dobnik and colleagues have illustrated that removing host cell DNA residues by incubating AAV samples with DNase I prior to quantification significantly decreased the titer [70].

### 2.2.2 Empty and full capsid ratio determination with TEM, AEX-HPLC and AUC

Empty capsids make up a significant amount of all capsids produced during AAV biosynthesis [71]. Up to 90% of all capsids can be empty [55]. Reasons for the presence of empty capsids are unknown. Supposedly cell culture conditions such as pH, ion type and concentration will affect the ratio [72]. However, empty capsids are undesirable since they do not mediate a therapeutic effect. By binding to the cellular receptors they even could decrease the uptake of full viral vectors and might trigger an immune response [73]. In the next section, different methods for quantifying the ratio of full and empty capsids are elaborated.

Transmission electron microscopy (TEM) is a widely used technique for defining empty and full capsid ratios. AAVs are visualized by a so called “negative-staining” with uranyl acetate. Full particles exclude the dye and therefore appear as white dots. Whereas empty capsids take up the dye and appear with a dark spot in their capsid [74]. TEM has several advantages such as a minimal sample consumption and a direct visualization. Nevertheless, it is time consuming, expensive, arbitrary and dependent on the sample preparation [73].

Capsids containing DNA have slightly different surface charges, which allow a sequential elution with anion exchange chromatography (AEX). The capsid elution can be detected by either fluorescence or UV absorbance. Such measurements allow rapid, simple and highly reproducible determination of full and empty capsid ratios [75]. High throughput, QC-friendliness, automation of the measurement and small sample volumes are the big advantages of anion exchange chromatography methods [76]. Normally, viral formulations contain a low amount of protein concentration, which cause low signal-to-noise ratios. By using a fluorescence detector, the signal-to-noise ratio can be improved. Despite such advances, AEX strategies only allow rough estimations of the full and empty capsid ratios [73].

A third method for quantifying full and empty capsid ratios is Analytical Ultra Centrifugation (AUC). AUC is considered as a gold standard for defining the ratio of empty and full capsids [76]. The presence of a vector genome increases the density and thus enables separation over sedimentation velocity from empty capsids [73]. This method is a very accurate and reproducible tool for quantifying empty and full capsids [77]. Traditionally a Cesium-Chloride density gradient AUC was applied to separate empty and full capsids, however this approach is not scalable [71]. Recent advances in Ultracentrifugation and computational methods enable a real-time monitoring of sedimentation and a subsequent quantification of AAV capsids using an UV detection system [78]. Its high sensitivity even enables a separation and quantification of capsids with different genome sizes [78]. A big limitation, especially for research purposes, is the requirement for high volumes of at least 400 μl and high titers of around 5·10¹² vg/mL [73].

### 2.2.3 Number of Total Capsids with Capsid-ELISA and NTA

Previously mentioned strategies mainly focus on quantification of DNA-containing vectors. There is a lack of approaches for quantifying the total number of AAV capsids. Capsid-ELISAs are one of the only approaches to directly quantitate the total number of AAV capsids [79]. Serotype 1, 4, 5 and 6 specific antibodies can be used to perform a sandwich ELISA to quantify the respective total number of capsids [80]. Major drawbacks of ELISA assays are their price, high variability and the possibility of unspecific binding to free proteins [55].

A more recent publication by Kondratov and colleagues used nanoparticle-tracking analysis (NTA) to count the total number of AAV capsids. To gain a detectable signal, capsids were labeled with gold-nanoparticles [81].

### 2.2.4 Activity and Potency of Vector Formulations with Cell Based Expression Assays

AAVs are able to transduce dividing and non-dividing cells [52]. Transduction efficiency of AAV vectors is identified by measuring transgene expression [82]. This is an important quality attribute of a vector formulation since vector instability and degradation cause a reduction of transduction [27]. Mainly vectors carrying a transgene encoding for fluorescent proteins such as green fluorescence protein (GFP) are used in transduction assays [55]. Vector concentrations in cell-based assays are expressed as multiplicity of infection (MOI). MOI is the number of genome containing vectors that are added to one cell [83].

Transduction experiments are performed either in vivo, ex vivo or in vitro [84]. In in vitro experiments it is crucial to choose a cell line with high transduction efficiencies, since AAV serotypes have a distinct tissue tropism [4]. Many in vitro studies use HEK 293 cells for transduction experiments. This is an established well-known cell line and is also used for the biosynthesis of AAVs [85].

Ellis and colleagues performed a transduction study in which they screened a variety of different cells and serotypes. Experiments have shown that many progenitor cells were not well transduced and are not suited for in vitro AAV transduction assays. Whereas many immortalized human cell lines were highly transduced, especially by AAV1 and 6 [84].

When looking closer at the frequently used serotype 2 (AAV2), several publications describe successful transduction experiments of U2OS cells. These osteosarcoma cells are very susceptible to AAV2 infections. However, it is still unknown if this is caused by their mutations or their bone-cell-like nature [86]. Because of its high transduction by AAV2, the cell line was utilized in experiments identifying the earlier mentioned KIAA0319L receptor [42]. Ellis and colleagues measured an AAV2 transduction efficiency of 98% in U2OS cells, which corresponds to the transduction efficiencies observed in the other above mentioned publications [84].

Most of the cited literature uses flow cytometry to quantify the transduction efficiency. Flow cytometry is well suited for a rapid and robust quality assessment of a vector mediated gene transfer [87].

### 2.2.5 Particle Size Assessment with DLS and NTA

Dynamic light scattering (DLS) measures the light scattering of particles under Brownian motion. Bigger particles have a higher correlation due to slower movements than smaller particles and vice versa [88]. Based on that, DLS calculates the diffusion coefficient of the respective particles, enabling the calculation of the hydrodynamic radius [89].

Nano-tracking analysis is a more novel approach and based on the same principle as DLS. A laser visualizes light scattering under Brownian motion. A digital camera tracks, counts and measures the particle size. NTA has a higher resolution than DLS and is less viable to sample impurities [90]. Despite its advantages over DLS it is rarely used for size determination of AAVs.

DLS is highly sensitive and requires low sample volumes [30]. Therefore it became popular for size and aggregation measurements of viral vectors [89]. Vector aggregation might occur during purification and thus compromise the safety and potency profile of a viral therapy [91]. Consequently, DLS can be used as a tool for quality control after vector purification [92]. Most investigations use DLS for vector size and aggregation analysis [90]. Wang and colleagues, for example, investigated the effects of AAV capsid modifications on its size with DLS [34]. Other studies used DLS to investigate how antibodies can mediate vector aggregation which impacts tissue interactions [93]. Aggregation can also occur during storage of viral vectors. Therefore it is crucial to not only improve purification strategies but also focus on new innovative formulation approaches to keep the vectors stable [91]. For this application, DLS was used to assess the influence of different excipients on the AAV2 aggregation. Aggregation measurements revealed that the AAV2 aggregates decrease in size, when sufficient ionic strength was added to the formulations [30].

### 2.2.6 Assessment of Colloidal Stability with Zeta Potential

Zeta potential measures the surface potential of particles and is an important tool for characterizing colloidal stability of nano particles [94]. Zeta potential is mainly influenced by the pH. Nano particles such as viruses show a decreased colloidal stability when the pH is close to their isoelectric point. Nevertheless, ionic strength as well as particle concentration also affect the zeta potential [95]. Surface potential measurements can be applied during formulation development of adeno-associated viruses. According to the literature, AAV2 has a surface potential of -9.4 mV [96]. Colloidal stability is reflected by the magnitude of the zeta potential. Values below -30 mV or greater than +30 mV indicate a high degree of stability. Zeta potentials that are bigger than -25 mV or smaller than +25 mV tend to aggregate, flocculate or coagulate due to formation of van der Waals, hydrophobic interactions as well as hydrogen bonding [97].

### 2.2.7 Differential Scanning Fluorimetry as a Useful Tool in Formulation Development

Differential scanning Fluorimetry (DSF) is a fluorescence-based assay that is able to determine the thermal stability of proteins. The analytical method either detects an intrinsic or extrinsic fluorescence change. Intrinsic fluorescence is caused by aromatic amino acids such as tryptophan, phenylalanine and tyrosine. Upon temperature-induced protein unfolding, the amino acids change their location, which results in a change of the fluorescence spectra. Meanwhile extrinsic fluorescence is caused by the addition of an external dye [98]. SYPRO-orange is the most commonly used dye for DSF, due to its favorable signal-to-noise ratio [99]. Therefore, most experiments on the thermal stability of AAVs use SYPRO-orange. It binds to hydrophobic regions that become accessible upon capsid unfolding. A disadvantage of SYPRO-orange that limits its use in formulation development is its affinity to frequently used surfactants. Binding to the hydrophobic regions of these surfactants results in high background fluorescence [100]. Thus, newer approaches utilize the intrinsic fluorescence of AAV capsids as a simple and accurate alternative to SYPRO orange DSF [101]. Besides these improvements, low sample consumption, rapid, robust and cost-effective measurements are major advantages of DSF [4].

DSF manifested itself as a very valuable method in formulation development. As previously mentioned, it is able to identify serotypes by measuring their capsid melting temperatures [32]. A recent study has shown that DSF applications can go beyond the identification of AAV serotypes. Protein impurities for example significantly affect the DSF fingerprints and enable it to assess AAV batch purity. Likewise, the fluorescence signal intensity is directly proportional to the capsid concentration. Both mentioned applications can be utilized to assess batch-to-batch consistency of AAV preparations [102].

Bennett and colleagues performed a buffer screen and therefore illustrated another formulation related implementation of DSF. They measured how buffers affect the thermal stability of AAVs. Results showed that buffer selection influences the melting temperature of capsids in a serotype-specific manner. In the case of AAV2, Tris buffer compared to phosphate buffer increased the melting temperature up to 15° C. [103]. Not only the buffer, but also its pH, affect the thermal stability in a serotype-specific manner. The thermal stability of AAV5 proportionally decreases with decreasing pH. Whereas AAV2 has the highest thermal stability at a pH of five [102]. The above-mentioned publications highlight new formulation-related DSF applications.

### 2.3 Structure of Project and Formulation Study

The Project is divided into two parts. For the first few months, the focus was on evaluation and development of analytical methods for characterization of AAV2. First, a qPCR method was modified to make a dose estimation. In parallel, a cell based transgene assay was developed to test the expression induced by the formulations. Particle analysis was conducted and focused on zeta potential measurements, sub visible particle formation with light obscuration as well as on dynamic light scattering. Additionally, DSF techniques were established and applied to the AAV2 formulations.

In the second part of the Project, eight different formulations were evaluated in a three month stability study. Formulations in the literature, along with those provided by AAV suppliers and in marketed formulations such as Luxturna use a phosphate buffer supplemented with 0.001% P188. This buffer was used as a starting point for formulation development. Further formulations were developed by only changing one variable per formulation. This allowed for conclusions about the impact of the changed parameter. Different buffers, pH and excipients were used to assess their impact on the AAV2 stability. Bennett and colleagues have already shown that the pH directly impacts the thermal stability. Thus, different pH ranges were tested and pHs of 5.5, 6.8 and 7.5 were chosen. The lowest pH of 5.5 not only resulted in the highest AAV2 thermal stability, it also mimicked the endosomal environment [103]. Subsequently, three popular and well-established formulation buffers were selected that buffer in the selected pH ranges and are suited for freeze-drying. The same buffer concentrations were chosen to allow for a head-to-head comparison. 20 mM L-histidine, sodium citrate and sodium phosphate were chosen as formulation buffers for the AAV2 stability study. The latter is a popular AAV storage and shipping buffer, whereas L-histidine buffers are commonly used in formulation of biologics. Lastly, a sodium citrate buffer was selected as, amongst other things, its impact on AAV stability is not described in the literature. All formulations were supplemented with 290 mM sucrose. Sucrose was required as a bulking agent for freeze-drying. The study tested two different surfactants. On the one hand, Kolliphor P188 was tested as a frequently used surfactant in AAV formulations and on the other hand, Polysorbate 80 was tested. Kolliphor P188 and PS80 concentrations of 0.001% and 0.02%, respectively, were used. These are standard surfactant concentrations described in the literature, used by the AAV supplier and in marketed AAV formulations. Such low surfactant concentrations were shown to be sufficient for preventing non-specific binding to glass or plastic surfaces [104].

A central aim of this Project was to evaluate the impact of freeze-drying on the stability of AAV2 vectors. Freeze-drying would present an attractive alternative to the current AAV storage and shipment temperature of −80° C. Due to the high priced AAV vectors, it was decided to apply freeze-drying to four formulations. Although AAVs are known to be stable, they tend to aggregate.

Wright and colleagues proposed different mechanisms responsible for aggregation. In high titer formulations with titers bigger than 1014 vg/mL symmetric capsid structure and interactions between oppositely charged capsid pockets reduce AAV solubility. Since less vector aggregation is seen in AAV2 samples treated with a nuclease, they proposed that residual host cell DNA fragments bind over electrostatic interactions to the AAV2 capsids and mediate aggregation. Ionic strength is described to prevent such vector aggregates [30]. Because this study focused on freeze-drying novel, non-salt-based excipients that address the problem of aggregation and simultaneously could be freeze-dried were evaluated.

One formulation was provided with 0.2 mg/mL sodium hyaluronate. The use of hyaluronic acid in AAV formulation development is not yet described in the literature. Another formulation used 1% human serum albumin. Albumin supposedly interacts with the AAV capsid and increases its transduction efficiency [105]. These excipients were selected because they do not interfere with freeze-drying. Table 2 summarizes all formulation compositions. Because AAV2 vectors are very high priced, it was decided to use the smallest titer which still enabled the implementation of all chosen analytical methods evaluated in the first part of this Disclosure. Thus the formulation study was conducted with an AAV2 titer of 1·10¹¹ vg/mL.

TABLE 2
Formulation compositions used for the formulation stability study, all formulations
aimed for a titer of 1.10¹¹ vg/mL

| Formulation | Buffer | Stabilizer 1 | Stabilizer 2 | Surfactant | pH |
|---|---|---|---|---|---|
| Formulation 1 | 20 mM L-Histidine | 290 mM Sucrose | — | 0.02% PS80 | 5.5 |
| Formulation 2 | 20 mM L-Histidine | 290 mM Sucrose | | 0.02% PS80 | 6.8 |
| Formulation 3* | 20 mM Sodium-Citrate | 290 mM Sucrose | | 0.02% PS80 | 6.8 |
| Formulation 4* | 20 mM Sodiumphosphate | 290 mM Sucrose | | 0.02% PS80 | 6.8 |
| Formulation 5 | 20 mM Sodiumphosphate | 290 mM Sucrose | | 0.02% PS80 | 7.5 |
| Formulation 6* | 20 mM Sodiumphosphate | 290 mM Sucrose | 1% | | 6.8 |
| | | | Albumin | | |
| Formulation 7* | 20 mM Sodiumphosphate | 290 mM Sucrose | 0.2 mg/mL | | 6.8 |
| | | | Sodium | | |
| | | | hyaluronate | | |
| Formulation 8 | 20 mM Sodiumphosphate | 290 mM Sucrose | | 0.001% P188 | 6.8 |

*Formulations that were additionally freeze-dried

Experiments were performed to compare the impact of different temperature expositions on the stability of AAV2. Thus a broad range of temperature ranges was added to the study. Firstly, it was desirable that two different freezing temperatures, such as −20° C. and −80° C. and their influence on the AAV2 stability were compared. Secondly, elevated storage temperatures of 2-8° C., 25° C. and 40° C. were investigated. Simultaneously, formulations were exposed to several stresses such as freeze-thaw cycles to −20° C. and −80° C. as well as exposure to horizontal agitation stress. The latter present a condition that it is yet not described in the literature. However freeze-thaw stress was described in several publications. Croyle and colleagues showed a decrease in vector gene expression when exposed to freeze-thaw cycles [27]. At low titers, such cycles were described to induce vector aggregation [30]. Because both characteristics were investigated, this stress condition presented an interesting condition. Since AAV2 were high priced, only limited amounts of vectors were available and it was decided to limit freeze-drying to four formulations. Based on some literature evidence, it was expected that exposure to 40° C. would quickly result in a loss of infectivity to save material. Therefore, the formulations were only exposed for two weeks [28]. The stability of formulations exposed to the other temperatures was investigated for three months. All formulations were analyzed at TO directly after manufacturing as well as after three month of storage, but no TO analysis of freeze-drying was conducted to save material. For later analyses condition specific pull points were set to save material. The detailed pull point setup is illustrated in Tables 3 and 4.

TABLE 3
Pull point setup for formulation study, X = Pull points conducted

| Storage | | Pull Points | | | |
|---|---|---|---|---|---|
| temperatures | | T0 | Two weeks | One month | Three months |
| −80° | C. | X | | | X |
| −20° | C. | X | | | X |
| 2-8° | C. | X | | X | X |
| 25° | C. | X | X | X | X |
| Freeze-dried, | | X | | X | X |
| 2-8° | C. | | | | |
| Freeze-dried, 25° C. | | X | | X | X |
| 40° | C. | X | X | | |

TABLE 4
Stress conditions investigated in the formulation study
Stress conditions

| | |
|---|---|
| Freeze-thaw to −80° C. | 10 cycles to room temperature |
| Freeze-thaw to −20° C. | 10 cycles to room temperature |
| Horizontal Agitation | Five days, 200 rpm at 2-8° C. |

### 2.4 Aim of Project

The aim of this Project is to evaluate and develop analytical methods used for characterization of AAV2 vectors followed by performance of a three-month stability study with AAV2.

Evaluated analytical methods aimed to characterize the dose as well as efficacy. On the other side, physico-chemical characterizations will be performed to get information about the aggregation behavior, formation of sub visible particles, surface potential and thermal stability of AAV2 vectors.

Optimized or developed analytical methods will then be applied in a three month formulation stability study. During this study the goal was to characterize the impact of different excipients, pH ranges, buffers and surfactants on the stability of AAV2 vectors. A central goal was to evaluate the impact of freeze-drying on the vector stability. In the stability study, formulations will be exposed to different temperatures as well as to different stresses, such as horizontal agitation stress as well as freeze-thaw stress.

### 3 Materials

### Buffer Manufacturing and Compounding

L-Histidine monohydrochloride (J.T Baker), L-Histidine (J.T Baker), Citric acid 1 H₂O (J.T Baker), Trisodium citrate 2 H₂O (J.T Baker), Sodium phosphate monobasic 1 H₂O (J.T Baker), Sodium phosphate dibasic 2 H₂O (J.T Baker), Sucrose (Pfanstiehl), Polysorbate 80 (J.T. Baker), Kolliphor P188 BIO (BASF), Sodium Hyaluronate (Lifecore Biomedical), Albumin human (Sigma-Aldrich), pH-Meter 780 (Metrohm), 2R/13 mm Schott Vials SL/NBB Tubular Fiolax Clear Type I (Schott), 13 mm Serum grey Fluorotec stoppers WES (Lonza DPS), 13 mm 1 yo grey Flurotec-stoppers-WES (Lonza DPS), Whatman Pursdisc PVDF syringe filter, 0.2 μm, 13 mm (GE Healthcare)

### Lyophilisation and Karl-Fisher Titration

Lyostar 3 (SP Scientific), Safety cabinet (A1 safetech), Karl-Fisher Titrator (Metrohm 3.0)

### qPCR

1000×AAV Standard (Virovek Inc), AAV2-CMV-GFP (Virovek Inc), Forward Primer 100 μM (Sigma-Aldrich), Reverse Primer 100 μM (Sigma-Aldrich), powerUp, SYBR Green Master Mix (appliedBiosystems), Nuclease-free water (ThermoFisher), DNase I, RNase-free (ThermoFisher), 10× DNase I buffer+MgCl₂ for DNase I (ThermoFisher), 50 mM EDTA (ThermoFisher), Thermoblock (Eppendorf), Quantstudio 5 (appliedBiosystems), MicroAmp, EnduraPlate optical 96-well (appliedBiosystems),Optical adhesive covers (appliedBiosystems), RNase AWAY (Molecular Bio Products)

### TEM

Transmission electron microscope Philips CM100 (Philips), 2% Uranyl acetate (Science Services), Copper Grid (Graticules Optics), AAV2-CMV-GFP (Virovek Inc)

### DLS/MADLS and Zeta Potential

Zetasizer Ultra (Malvern Instruments), High precision quartz cell 10×10 mm light path (Hellma Analytics), Nanosphere size standard 50 nm (ThermoScientific), Nanosphere size standard 60 nm (ThermoScientific), Nanosphere size standard 100 nm (ThermoScientific), DTS 1070 cuvette (Malvern Instruments), zeta potential transfer standard DTS1235 (Malvern Panalytical)

### DSF

Optim Unit (Unchained labs), SYBR-Gold nucleic acid stain (appliedBiosystems), SYPRO-Orange (Sigma-Aldrich)

### Amplification and Subcultivating of U2OS Cells

U20S-HTB-96 (ATCC), McCoy's 5A Medium (Gibco), Fetal Bovine Serum (Gibco), penicillin-streptomycin (Gibco), PBS (Gibco), Nunc Easy flasks 75, 175 and 225 cm² Nuclon deta surface (ThermoScientific), 0.25% trypsin-EDTA (1×) (Gibco), CO₂ Incubator (Binder), Vial-cassette (Chemometec), Nucleocounter NC-200 (Chemometec), Isothermal V1500-AB Series (Labtec), Shaking waterbath GFL 1086 (FAUST), Centrifuge 5920R (Eppendorf)

### Cell Viability Assay

AlamarBlue cell viability reagent (Invitrogen), Nunc F96 Microwell black (ThermoScientific), Vial-cassette (Chemometec), Nucleocounter NC-200 (Chemometec), Spectramax id3 (Molecular devices), McCoy's 5A Medium (Gibco), CO₂ Incubator (Binder)

### Transgene Expression Assay

rGFP 1 mg/mL (Roche), Nunc F96 Microwell black (ThermoScientific), Vial-cassette (Chemometec), Nucleocounter NC-200 (Chemometec), Spectramax id3 (Molecular devices), McCoy's 5A Medium (Gibco), CO₂ Incubator (Binder), U2OS-HTB-96 (ATCC), AAV2-CMV-GFP (Virovek Inc.), PBS (Gibco)

### Filter Experiment

13 mm Puradisc syringe filter, 0.2 μm, PVDF (GE Healthcare), 10 mm Anotop syringe filter, 0.2 μm PVDF (GE Healthcare), 13 mm Millex syringe filter, 0.2 μm, PVDF (Merck Millipore), Quantstudio 5 (appliedBiosystems) and all above mentioned qPCR equipment.

### 4 Methods

### 4.1 Evaluation and Development of Analytical Methods for Characterization of AAV2

Most methods were tested before they were applied to the formulation studies. During the evaluation and development of analytical methods it should be evaluated if the methods are applicable to AAV2 and if modifications are required. During this study, some methods such as qPCR were extensively modified and others such as the GFP expression assay were newly developed. Experiments performed as part of the analytical method evaluation and development are further elaborated upon in the next section.

### 4.1.1 Quantification of AAV2 Inverted Terminal Repeats (ITR) with Quantitative Polymerase Chain Reaction (qPCR)

This universal ITR quantification approach was used because it is well described and frequently used in the literature as well as by the AAV2 supplier. Optimization and establishment of this universal AAV2 method allow for a transgene independent AAV2 titration to be in place which can be applied to a variety of different AAV2 projects. For titer quantification the universal AAV2 ITR qPCR originally developed by Aurnhammer and colleagues was further modified [65]. The qPCR method was modified by replacing the TaqMan approach with the more convenient and cost-efficient SYBR-Green approach. This modification was based on publicly accessible protocols from Addgene. The SYBR-Green based AAV2-ITR qPCR was further supplemented with a DNase I digestion step, as well as with a capsid-opening step. Experiments were performed with and without these digestion and opening steps to estimate the impact of these treatments.

The following sequences were used as Primers [65]:

Forward Primer (FW):
(SEQ ID NO: 1)
5′-GGAACCCCTAGTGATGGAGTT-3′
Reverse Primer (RW):
(SEQ ID NO: 2)
5′-CGGCCTCAGTGAGCGA-3′

### 4.1.2 AAV2 Titration Procedure

Initially a dilution series with the purchased linearized plasmid standard was prepared. The resulting standard curve served as a quantification tool during the titration. Five dilutions were made with nuclease-free water. The obtained 1000× Standard with a concentration of 1.0·10⁸ DNA amplicons per μL was diluted to required concentrations (1·10⁷, 1·10⁶, 1·10⁵, 1·10⁴, 1·10³ DNA amplicons/μL). Throughout all qPCR experiments, the standard curve was prepared in this manner.

Subsequently, AAV samples were prepared with either a DNase I digestion and capsid opening step or without. All pull point analysis during the formulation study 1 and 2 contained such a digestion and opening step. Prior to these experiments, untreated samples were prepared with treated samples to detect the impact of such a treatment. Samples that included such treatment were prepared by mixing 5 μL of sample with a titer of 1·10¹¹ vg/mL with 38 μL of nuclease-free water, 5 μL of 10× DNase reaction buffer with MgCl₂ and 2 μL of DNase I (1 U/μL). The mixture was incubated at 37° C., for 40 minutes. After incubation, 10 μL of 50 mM EDTA solution were spiked to the mixture and the sample was heated up to 65° C. for 10 minutes to inactivate the DNase I. Then the capsids were opened at 95° C. for 30 minutes and diluted accordingly. Afterwards, these samples were diluted in nuclease-free water (1:10, 1:200, 1:1000, and 1:5000). Samples that were not opened nor digested with DNase I were directly diluted identical to the digested and opened samples. The qPCR plate was filled with 5 μL of each sample and standard dilution in duplicates. Additionally, two wells were filled with 5 μL of nuclease-free water serving as a no template control (NTC). The plate was sealed and the master mix prepared. The master mix recipe used for one well is composed of 4.7 μL nuclease-free water, 10 μL Power UP SYBR-Green master mix and 0.15 μL of 100 μM forward and reverse primer. A multichannel pipette was used to add 15 μL of master mix to each well. The plate was sealed with an optical adhesive cover, centrifuged for two minutes at 3000 rpm and transferred to the Quantstudio 5. The PCR running profile was adjusted to meet the requirements of the used master mix (50° C. for 2 min, 98° C. for 3 min, 40×98° C. for 15 sec followed by 58° C. for 30 sec). Analysis was finished with a three-step melt curve (95° C. for 15 sec, 60° C. for 1 min and 95° C. for 15 sec). QuantStudio design and analysis software was used for the data analysis.

### 4.1.3 Evaluation of AAV2 Sample Treatment for qPCR

The first experiment characterized the impact of the capsid opening and DNase I digestion step. First, 20 mM phosphate buffer containing 0.001% P188 was prepared. This buffer was used to manufacture a 1.2·10¹¹ vg/mL AAV2 suspension. Three Eppendorf tubes were provided with each 5 μL of that suspension. 45 μL of nuclease-free water was added to the first aliquot. The other aliquots were provided each with 38 μL of nuclease-free water, 5 μL of 10× DNase reaction buffer with MgCl₂ and 2 μL of DNase I (1 U/μL). Afterwards, capsids of two aliquots were opened as described above. The opening procedure was applied to the aliquot, which was diluted with only nuclease-free water and to one of the two aliquots containing DNase I. Capsids of the other aliquot containing DNase I were not opened. After opening, the samples were diluted and amplified according to the above-mentioned qPCR procedure.

### 4.1.4 Adhesion of AAV2 to Eppendorf tubes

Because all serial dilutions were done in Eppendorf tubes and many experiments used Eppendorf tubes in intermediate steps, an adhesion experiment was performed. An AAV2 formulation with a titer of 1·10¹⁰ vg/mL in phosphate buffer, pH 7.4 containing 0.001% P188 was prepared. 500 μL of this formulation was made and divided into two aliquots. Each aliquot was stored in a 1.5 mL Eppendorf tube for 24 h. After 24 h one aliquot was extensively mixed by pipetting up and down ten times with a volume of 100 μL whereas the other aliquot was not mixed at all. A qPCR of both aliquots was performed including a digestion and capsid opening step.

### 4.1.5 Quantification of Full and Empty Capsids with TEM

It was decided to use TEM for determination of the full and empty capsid ratio. Compared to AUC and AEX-HPLC, TEM requires much smaller volumes and titers. Even though AUC is known as the gold standard for quantifying full and empty capsids, several publications have shown that quantification with TEM directly correlated with results obtained with AUC. Since only full and empty capsids of the AAV2 stock suspension were quantified, no time intensive implementation of a high-throughput method such as AEX-HPLC was needed. Transmission electron microscopy (TEM) and the associated sample preparation were conducted in the Nano Imaging Lab at the University of Basel on a Philips CM100. Negative staining with 2% uranyl acetate enabled the differentiation of empty and full AAV capsids. Before adding the virus on the copper grid, the grid needed to be glow discharged. The copper grid was placed in the discharging chamber. A dome was installed over the copper grid, the needle valve was closed carefully and the vacuum pump was switched on. Subsequently, the power supply was turned up to 50 volts and the copper grid was glow discharged for 30 seconds. Followed by incubating, washing and staining the samples on the copper grid. During this Project two different sample preparations were tested. In the first experiment, Nano Imaging Lab's standard protocol for AAV negative staining was applied. Therefore, the copper grid was incubated for one minute with 10 μL of a 1·10¹⁰ vg/mL sample. Following the incubation, the grid was washed three times with 50 μL ddH₂O. After each washing step, the water was removed using a filter paper. Negative staining was achieved by incubating the copper grid two times for ten seconds with 5 μL of a 2% uranyl acetate solution. Only a few capsids were detected using this sample preparation. That is the reason for the modification and repetition of the sample preparation procedure. In the second staining attempt, glow discharging was executed identically to the first time. The discharged copper grid was then incubated with 15 μL of a 1·10¹¹ vg/mL sample. After this, only one washing step with 50 μL ddH₂O was executed. The number of staining steps with 2% uranyl acetate was kept the same as above. For quantification of the empty and full capsid ratio, 11 random pictures were taken. One picture was taken with a voltage of 20 kV and one with 37 kV. The other nine pictures were taken with a voltage of 11 kV.

### 4.1.6 Particle and Aggregation Analysis with DLS and MADLS

DLS was used to monitor the particle size distribution in the nanometer range. This is the state of the art for measuring and quantifying viral vector aggregation. Its small sample consumption makes it an attractive method. As previously mentioned, the titer used was 1·10¹¹ vg/mL. This titer corresponds to a very low protein concentration of around 0.62 μg/mL. During method evaluation it was investigate if these concentrations could be detected. All DLS measurements were conducted in a low volume, high precision ZEN 2112 quartz cuvette on a zetasizer Ultra from Malvern Instruments. First, a system suitability test (SST) with latex-beads was executed. A 10 mM NaCl solution was prepared, sterile filtered and aliquoted into four sterile 15 mL tubes. Each tube was provided with two drops of either the 50, 61, or 100 nm latex bead standards. The suspensions were carefully homogenized by gently inverting the flasks. The high precision ZEN2112 quartz cuvette was provided with 50 μL of each standard. Polystyrene latex bead settings with a refractive index of 1.59 and an absorption of 0.01 were selected to conduct the SST. Water was chosen as a dispersant with a refractive index of 1.33 and a viscosity of 0.8872 mPas. The acceptance criteria for the Z-Average of the 50 nm latex bead standard was 48 nm±3 nm. For the 61 nm standard, it was 61 nm±4 nm and for the 100 nm standard it was ±8 nm. Before loading samples into the low volume quartz cuvette type ZEN2112 it was rinsed with water and ethanol. Pressurized, particle free air was used to dry the cuvette. Then AAV2 vectors were diluted in PBS, pH 7.4 supplemented with 0.001% P188 to a titer of 1·10¹¹ vg/mL. The cuvette was filled with 50 μL of the sample. Measurements were performed using AAV2 settings. This measurement method consists of a refractive index of 1.45 and an absorption of 0.001. Water was chosen as the dispersant with a refractive index of 1.33 and a viscosity of 0.8872 mPas. The temperature was set to 25° C. and equilibration time was set to 120 seconds. Dispersant scattering was set to 75 kcps. Each sample was characterized by three back scatters. Triplicates of MADLS measurements were applied to all formulations. MADLS measurements were also applied to placebos at T0, after Freeze-Thaw cycles, agitation stress, storage of two weeks at 40° C. and storage of freeze-dried samples for one month at 2-8° C. After the analysis, the sample was transferred to an Eppendorf tube and stored at 2-8° C. for further analysis.

### 4.1.7 Zeta potential measurement

Zeta potential was measured with the diffusion barrier method in a DTS 1070 cuvette on a zetasizer Ultra from Malvern Instruments. A system suitability test (SST) was performed by filling a DTS 1070 polystyrene cuvette with a −42 mV±4.2 mV zeta potential transfer standard. Two thermal contact plates were attached to the cuvette. The standard was measured with instrument settings for polystyrene latex beads, measured with a refractive index of 1.59 and an absorption of 0.01. Water was chosen as a dispersant. After the system suitability test was passed, cuvettes were flushed with water and filled with PBS, pH 7.4, supplemented with 0.001% P188. Samples were added to the cuvette by using the diffusion barrier method (DBM). This method was specifically developed for low sample volumes. With help of a gel electrophoresis-loading tip, 130 μL of sample was loaded on the bottom of the cuvette. The AAV2 formulation used was prepared in PBS, pH 7.4 supplemented with 0.001% P188 and had a titer of 1·10¹¹ vg/mL. Thermal conduct plates were attached to the cuvette. Samples were measured with instrument settings for proteins, which were a refractive index of 1.45 and an absorption of 0.001. Water was chosen as a dispersant. All measurements were performed in triplicates. Afterwards the cuvettes were flushed with water and ethanol and reused at least two times.

### 4.1.8 Cell Line Amplification

Cell culture medium was prepared upon receiving the cell line. McCoy's 5A medium was provided with fetal bovine serum (FBS) and penicillin-streptomycine (Pen/Strep) to receive a final concentration of McCoy's 5A medium containing 10% FBS and 1% Pen/Strep.

One cryovial containing around 1·10⁶ U20S-HTB-96 cells was obtained from ATCC. Cells were thawed and suspended in 9 mL of preheated medium. The tube was centrifuged at 136 rcf for 5 min, and the medium was aspirated. Afterwards the cell pellet was resuspended in 16 mL of preheated medium and seeded in a 75 cm² culturing flask. After a confluency of around 90% the adherent cells were suspended and provided with 8 mL of preheated medium. After centrifugation at 136 rcf for 5 min, the supernatant was aspirated and the pellet was resuspended in 4 mL of medium. Two 225 cm² culturing flasks containing 48 mL of preheated medium were each provided with 2 mL of suspension and stored at 37° C., 5% CO₂. After reaching 90% confluency, cells from one culturing flask were suspended and counted. After washing the cells, a 1.2·10⁶ cells/mL cell suspension (Passage 2) was prepared in medium that was supplemented with 5% (v/v) DMSO. Seven cryo tubes were provided with 1 mL of suspension (Passage 2) and frozen in liquid nitrogen. Cells from the other flask were further amplified by suspending them in 4 mL of medium and transferring 1 mL of suspension to four 175 cm² culturing flasks. At a confluency of 90% the cells were harvested, counted and resuspended in medium supplemented with 5% (v/v) DMSO. Another 30 cryotubes with a U2OS concentration of 1.6·10⁶ cells/mL (Passage 3) were frozen in liquid nitrogen.

### 4.1.9 Sub-Culturing Procedure

Sub-culturing was always conducted in 75 cm² flasks. Old medium was aspirated and the adherent cells were washed with 12 mL of preheated PBS. After aspirating the phosphate buffer, 2 mL of 0.25% Trypsin, 0.03% EDTA solution were added to the flask and the flask was incubated 37° C. for three minutes until the cells detached. Detached cells were suspended in 8 mL of medium and centrifuged for five minutes at 136 rcf. The supernatant was aspirated and the cell pellet was resuspended in 5 mL of medium. A new 75 cm² cell culturing flask filled with 16 mL of preheated medium was then provided with 1 mL of this suspension and stored in the incubator at 37° C. and 5% CO₂. Sub-culturing was initiated if the cells reached a confluency of 90%. Each cell line was sub-cultured until they reached passage 30.

### 4.1.10 Cell Line Establishment and Cell Viability Assay

Prior to cell based transgene expression assays, the cell line was established by performing an alamarBlue cell viability assay. This assay was performed to obtained information about the ideal seeding densities, resulting grow rates and cell viabilities. U2OS cells were harvested and suspended. A stock suspension was prepared with a concentration of 8.2·10⁶ cells/mL. A dilution series was prepared with different cell concentrations (4.1·10⁵, 2.0·10⁵, 1.0·10⁵, 5.0·10⁴, 2.5·10⁴ and 1.25·10⁴ cells/mL. Exactly 100 μL of each suspension was added to a 96 well plate to receive cell densities of 4.0·10⁴, 2.0·10⁴, 1.0·10⁴, 5000, 2500 and 1250 cells/well (3906, 7813, 1.56·10⁴, 3.13·10⁴, 6.25·10⁴, 1.3·10⁵, 2.63·10⁵ cells/cm²). Each density was plated in eight replicates. The plate was stored over night at 37° C., 5% CO₂. After 24 h of incubation, the first cell viability assay was performed by aspirating the old medium and replacing it with 180 μL of fresh medium. 20 μL of alamarBlue reagent was added to obtain a 10% (v/v) alamarBlue-medium solution. The plate was incubated for two hours and 45 min at 37° C. and 5% CO₂. After incubation, 100 uL of the supernatant from each well was transferred to a black 96-well plate. Fluorescence was measured at 550 nm excitation and 590 nm emission. To obtain comparable results, the photomultiplier (PMT) within the Spectramax id3 was set to low. After this measurement, the residual supernatant was aspirated and replaced it with 150 μL of medium. The cell viability assay was repeated for seven days in a row. At day eight, the cells were killed by replacing the medium with 70% isopropanol. After an incubation of five minutes, the isopropanol was replaced again with medium and the assay was carried out as described above. This measurement acted as a negative control to show that fluorescence could only be obtained with viable cells.

### 4.1.11 Transgene Expression Assay Development

Most published literature uses either a fluorescence-activated cell sorting (FACS) device or a confocal microscope to quantify the transduced and transgene expressing cells. As neither a FACS sorter nor confocal microscope were available, a cell-based expression assay using a plate reader was developed. The disadvantage of this assay was the inability to quantifying the exact number of cells expressing GFP. Because the main interest was how the formulation induced transgene expression behaves compared to the starting expression at T0, these limitations were acceptable. This plate reader assay offered a high throughput, cost effectiveness and in-house availability.

After an in-depth literature search adherent U2OS-HTB-96 cells (BSL-1) were nominated as the cell line to be used for the development of a GFP expression assay. Even though the literature mainly describes HEK293 cells for such experiments, they were purposely not used, because they are not well transduced by AAV2 and are not strongly adherent. Both characteristics are mandatory for a successful plate reader assay and are fulfilled by U2OS cells. Because it was feared that there would be too low of a signal intensity, as many cells as possible were plated per well, without negatively impacting cell viability. An initial cell density of 10 000 cells/well appeared to be suited for the assay, since GFP expression could be monitored for four days before cell viability decreased. This cell number also enabled work to be performed in replicates.

First, four wells were provided each with 100 μL of a 1.0·10⁵ cells/mL cell suspension (1.0·10⁴cells/well). Directly after seeding, 100 μL of a 1·10¹⁰ vg/mL virus suspension was added to one well resulting in a multiplicity of infection (MOI) of 10⁵ and 100 μL of a 1·10¹¹ vg/mL virus suspension was added to the other well, resulting in a MOI of 10⁵. The other two wells were used as blanks and therefore provided with 100 μL of PBS. This plate was incubated at 37° C., 5% CO₂. After 24, 48, 72, 96 h the fluorescence was measured with an excitation and emission wavelength of 488 nm and 520 nm, respectively. The medium was not renewed nor exchanged for four days. After the 96 h measurement, the medium was aspirated and replaced with 100 μL PBS and the fluorescence measurement was repeated. After interpreting the results from the previous experiment a wavelength scan was performed to determine the excitation and emission wavelength of GFP. A 1 mg/mL recombinant GFP solution was prepared in a serial dilution in PBS (10 μg/mL, 1 μg/mL, 100 ng/mL, 10 ng/mL, 1 ng/mL). The wavelength scan was performed in a black 96-well plate. Each well was provided with 100 μL of the respective dilution. After determination of the ideal excitation and emission wavelengths, and optimizing the procedure, a MOI screen was performed. This screen was performed to obtain information on how to design the GFP expression assay within the formulation study. The goal was to define the MOI resulting in the lowest detectable fluorescence as well as to characterize the ability of the plate reader to detect fluorescence difference caused by different MOIs. The experiment additionally should highlight the impact of incubation time on the GFP expression. Four distinct virus samples were prepared with different titers. Samples had titers of 1·10¹¹ vg/mL, 1·10¹⁰ vg/mL, 1·10⁹ vg/mL, 1·10⁸ vg/mL. All dilutions were made with 20 mM sodium phosphate buffer, pH 7.4 containing 0.001% P188. U2OS-HTB-96 cells were harvested and suspended. The suspension was diluted to a concentration of 1·10⁵ vg/mL. 100 μL of the cell suspension was seeded into the wells to a cell density of 1·10⁴ cells/well. Freshly seeded cells were immediately supplemented with 100 μL of the respective virus dilution, which resulted in a MOI of 10⁶, 10⁵, 10⁴ and 10³ vg/mL, respectively. Each virus concentration was measured in five replicates. For each replicate well, a control well was also prepared. Control wells were filled with 100 μL of cell suspension. Instead of adding virus, 100 μL of 20 mM sodium phosphate buffer, pH 7.4 containing 0.001% P188 was added. After infection, the plate was stored in the incubator at 37° C., 5% CO₂. Before each fluorescence measurement at 24, 48, 72, 96 h, the medium was aspirated and replaced with 100 μL of phosphate buffer. Measurements were carried out on a Spectramax id3 at 460 nm excitation and 515 nm emission. After measuring, the phosphate buffer was again replaced with 150 μL medium. Pull Point analysis during formulation study 1 and formulation study 2 was conducted with a MOI of 10⁵ vg/cell and readouts after 72h were compared.

### 4.2 Formulation Study 1

TABLE 5
Formulation Buffers recipes used for dilutions during the pull
point analysis

| Buffer | Buffer | Stabilizer | Surfactant | pH |
|---|---|---|---|---|
| Buffer 1 | 20 mM L-Histidine | 290 mM Sucrose | 0.02% PS80 | 5.5 |
| Buffer 2 | 20 mM L-Histidine | 290 mM Sucrose | 0.02% PS80 | 6.8 |
| Buffer 3 | 20 mM Sodium citrate | 290 mM Sucrose | 0.02% PS80 | 6.8 |
| Buffer 4 | 20 mM Sodium | 290 mM Sucrose | 0.02% PS80 | 6.8 |
| | phosphate | | | |
| Buffer 5 | 20 mM Sodium | 290 mM Sucrose | 0.02% PS80 | 7.5 |
| | phosphate | | | |
| Buffer 6 | 20 mM Sodium | 290 mM Sucrose | — | 6.8 |
| | phosphate | | | |
| Buffer 7 | 20 mM Sodium | 290 mM Sucrose | — | 6.8 |
| | phosphate | | | |
| Buffer 8 | 20 mM Sodium | 290 mM Sucrose | 0.001% P188 | 6.8 |
| | phosphate | | | |

Buffer recipes were determined by using the Buffer and recipe formulary (B.A.R.F.), a program developed by R. J. Beynon and T. Patapoff. All buffers were made and sterile filtered through a 0.2 μm PVDF membrane when used for a pull point analysis (Table 5). During pull points, the buffers were used as diluents for zeta potential, sub visible particle measurements and transgene expression assays. Buffers used for pull point analysis were not provided with albumin nor sodium hyaluronate. All buffers were kept for up to two weeks protected from light and stored at 2-8° C.

The buffers used for compounding were prepared differently. Along with the buffers, a 2 mg/mL sodium hyaluronate, 5% PS80 and 1% P188 stock solution was made. Each stock was prepared with the respective buffer. Afterwards, the surfactants were spiked to a defined volume of buffer to receive the final surfactant concentration of 0.02% PS80 or 0.001% P188. Compounding was then started. This step was performed under sterile conditions in a Claire-Berner safety cabinet. Before starting with compounding, the Claire-Berner safety cabinet was extensively cleaned with isopropanol. Sterile gloves and sleeves were used when operating within the safety cabinet. In the first step, the AAV2 stock suspension was thawed, divided into eight aliquots and diluted with the respective buffer to a titer of 2.19·10¹² vg/mL. Then compounding was performed within sterile- and particle-free narrow-mouth bottles. First, the narrow-mouth bottles were provided with the buffers. Second, the pre-made excipient stock solution, such as albumin or sodium hyaluronate, was added to the buffers. Finally, pre-made AAV2 dilutions with a titer of 2.19·10¹² vg/mL were added to achieve a final titer of 1.095·10¹¹ vg/mL. The same procedure was applied for the placebo formulations that do not contain any AAV2. Each formulation was carefully mixed prior to aspiration with a silicon free 5 mL syringe. The needle was replaced with a whatman puradisc 0.2 μm, 13 mm syringe filter. The formulations were sterile filtered into new sterile- and particle-free narrow-mouth bottles. After sterile filtration, the Nalgene bottles were closed and the safety cabinet was cleaned again. In the next step, sterile and particle free 2 mL glass vials were provided with 245 μL of the formulations. 16 glass vials were filled if the formulations were additionally freeze-dried and 12 glass vials were filled if no freeze-drying was performed. All vials were manually stoppered with either sterile- and particle-free serum- or lyophilisation-stoppers. All vials not intended for freeze-drying were manually crimped and exposed to either different temperatures or stress conditions and stored for up to three months as described in Table 3. Exposure to freeze-thaw as well as agitation stress was started directly after manufacturing. Vials intended for freeze-drying were transferred to the freeze-dryer. Placebos that underwent lyophilisation were made in excess. On the lyophilsation shelf they were placed around the formulation vials to ensure a homogenous heat flux during the freeze-thaw process. Two placebo vials were provided with temperature sensors. One vial containing a temperature sensor was placed in the corner, meanwhile the other was placed in the center of the shelf After loading the vials on the shelf, it was transferred to the safety cabinet in front of the lyophilizer. Within this cabinet, the lyophilisation-stoppers were lifted with a sterile pincette. The shelf was then transferred into the lyophilizer and the electrodes were plugged-in. Freeze-drying was applied to four formulations and their placebos; namely formulation 3, 4, 6, 7. After freeze-drying, the vials were manually crimped and stored at different conditions (2-8° C. for 1 and three month or 25° C. for 2 weeks, 1 and three months). During pull point analysis, the formulations were analyzed using the methods described in the evaluation and development of analytical methods. Based on the results of these two measurements it was decided to abort the ongoing study and to start a second study with a modified compounding scheme.

### 4.3 Formulation Study 2

Formulation study 2 was prepared similar to study 1. The same buffer recipes and AAV concentrations were used. However, a small adjustment of the compounding procedure was implemented. Formulations were not sterile filtered. Otherwise, the compounding procedure was kept the same.

All methods were implemented as described during the method evaluation and development experiments. Seeding densities, cell viabilities and passages of the cells used in the transgene assay at the different time points are shown in Table 8 in the appendix. Additionally pH and sub visible particle measurements were performed. Prior to any sub visible particle measurement a system suitability test was performed with a 5 μm latex bead suspension. An 85 μL aliquot of the formulations was diluted to a titer of 1.0·10¹⁰ vg/mL in formulation buffer. Afterwards all samples were analyzed with a HIAC 9703+.

### 4.4 Additional Experiments

Additional experiments were performed to evaluate why AAV2 vectors showed low recovery after compounding in formulation study 1. The experiments were designed to observe if vector aggregation or affinity to the sterile filter membrane were responsible for vector loss.

It was also tested whether DSF can be applied to low concentration formulations with a titer of 1.0·10¹¹ vg/mL by using SYPRO-Orange or SYBR-Gold.

### 4.4.1 Impact of 150 mM NaCl on Sterile Filtration of AAV2

To find out if the titer loss during formulation study 1 was filter specific, the 13 mm Puradisc 0.2 μm PVDF was compared to a 13 mm Millex 0.2 μm PVDF and a 10 mm Anotop 0.2 μm PVDF filter. After contacting GE Healthcare, the Anotop filter was recommended for filtration of viral material. The two different filter sizes allowed a comparison of the impact of the filter surface area on the titer recovery after filtration. First, a solution of 100 mL of a 20 mM L-Histidine, 290 mM Sucrose, 0.02% PS80, pH 6.8 was made. Two 10 mL aliquots were taken and 88 mg of sodium chloride (NaCl) was added to one aliquot, resulting in a final NaCl concentration of 150 mM. 2985 μL was transferred from each aliquot to two Eppendorf tubes. Meanwhile, purchased virus stock with a titer of 2.19·10¹³ vg/mL was thawed. Each buffer aliquot was provided with 15 μL of the AAV2 stock. Samples were extensively mixed by pipetting up and down. Afterwards, the formulation that did not contain any sodium chloride was filtered. Filtration was performed through Puradisc, Anotop and Millex sterile filters. 1 mL of formulation not containing any sodium chloride was filtered through each filter. The same procedure was applied to the formulation containing 150 mM sodium chloride. Afterwards, the viral titer was determined using a standard qPCR protocol described above. Additionally, DLS measurements of the different samples were made.

### 4.4.2 Thermal Shift Assay with SYPRO-Orange and SYBR-Gold

Differential scanning fluorimetry (DSF) measurements were performed on the Optim system from Unchained labs. The experiment utilized SYPRO-Orange and SYBR-Gold to enhance signal intensity. SYPRO-orange and SYBR-Gold dyes and different concentrations were compared in this experiment. SYPRO-orange and the SYBR-Gold were purchased with a concentration of 5000× and 10 000×, respectively. For the experiment, the dyes were diluted in 20 mM L-Histidine, 290 mM Sucrose, 0.02% polysorbate 80, pH 6.8. Three dye stocks were made for SYPRO-Orange as well as for SYBR-Gold. Three μL of a 10×, 50× and 100× stock were produced and spiked to 27 μL of a 1.0·10¹¹ vg/mL AAV2 sample. This resulted in a final dye concentration of 1×, 5× or 10×. For each dye concentration, a buffer control with the same concentration was prepared. 9 μL of the stained AAV2 sample were added to the DSF capillaries. Each sample was measured in triplicate with the SYPRO-orange Optim settings. The starting and end temperatures were set to 15° C. and 95° C., respectively. The heating rate was set to 0.33° C./min.

### 5 Results

### 5.1 Results of the Evaluation and Development of Analytical Methods for Characterization of AAV2

### 5.1.1 Impact of DNase I Digestion and Thermal Capsid Opening Step on the Viral Titer

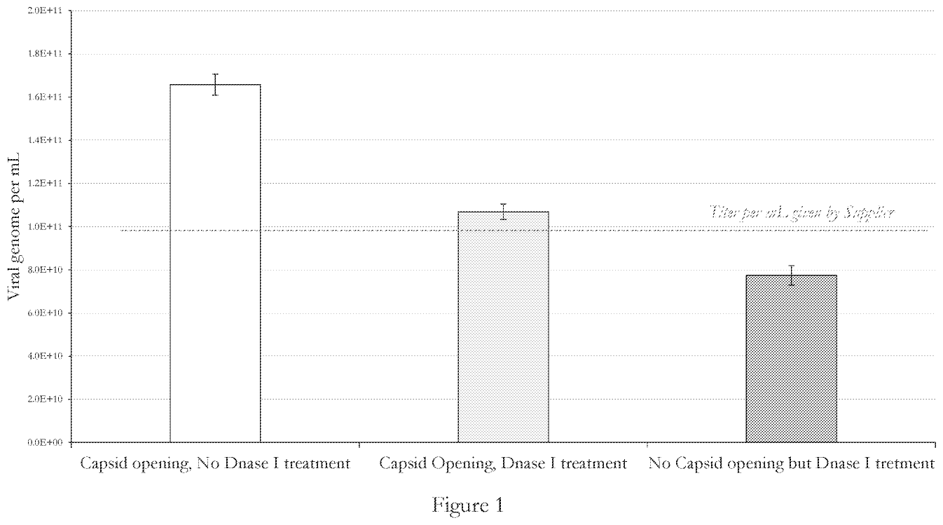

The aims of the qPCR method evaluation and modification were to obtain information about the influence of DNase I digestion and thermal capsid opening on titer quantification. FIG. 1 illustrates the titers obtained by three different treatments. The highest titers were obtained when the samples were opened before quantification. Addition of such a thermal capsid-opening step resulted in a titer of 1.66·10¹¹ vg/mL. Implementation of this procedure resulted in an overestimation of the titer given by the supplier. Addition of a DNase I digestion step led to a titer decline to 1.07·10¹¹ vg/mL. When performing only a DNase I digestion step, not followed by any thermal capsid opening, the titer decreased even more to 7.74·10¹⁰ vg/mL. Subtraction of titers resulting after digestion from not digested samples showed, that ˜5.9·10⁹ host cell DNA fragments/per mL are present in the AAV sample. This experiment showed that DNase I treatment prior to quantification significantly reduces the titer while a capsid opening step increases the titer. A combination of both sample treatments enable a precise titer determination with only a small deviation compare to the given titer from the supplier. Based on this result, titer quantification during the formulation studies applied a DNase I digestion and thermal capsid opening step. See FIG. 1.

### 5.1.2 Adhesion of AAV2 to Eppendorf Tubes

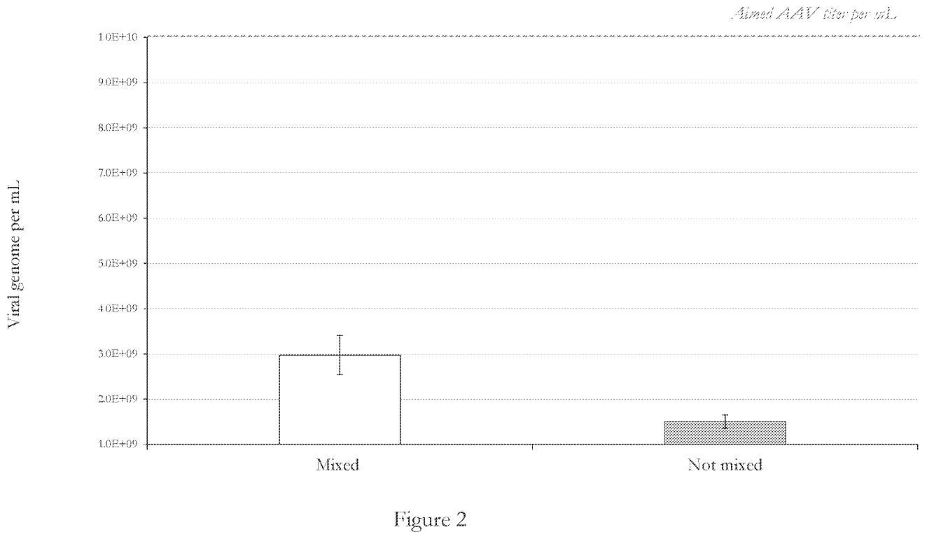

AAV2 formulations were frequently stored in Eppendorf tubes during the pull point analysis. Thus, an experiment was conducted to determine the adhesion of AAV2 to Eppendorf tubes upon storage for 24 h. FIG. 2 illustrates the AAV2 titers of mixed and not mixed samples after storage in Eppendorf tubes for 24 h. Sample mixing before quantification resulted in a titer of 2.9·10⁹ vg/mL and was twice as high compared to the sample that was not mixed measuring a titer of 1.5·10⁹ vg/mL. The data shows that extensive mixing is able to increase titers. Both measurements underestimated titers given by the AAV2 supplier. Based on these results, low binding tubes were used in the following experiments and samples were extensively mixed before use. See FIG. 2.

### 5.1.3 Full and Empty Capsid Ratio with Transmission Electron Microscopy

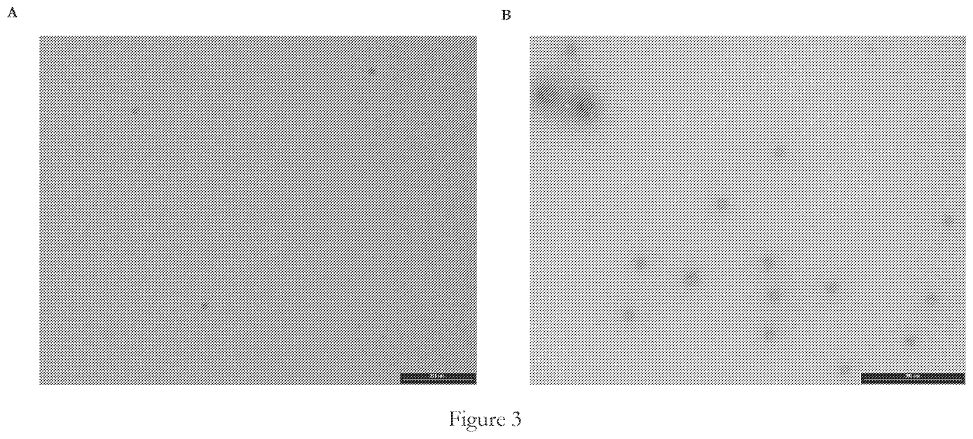

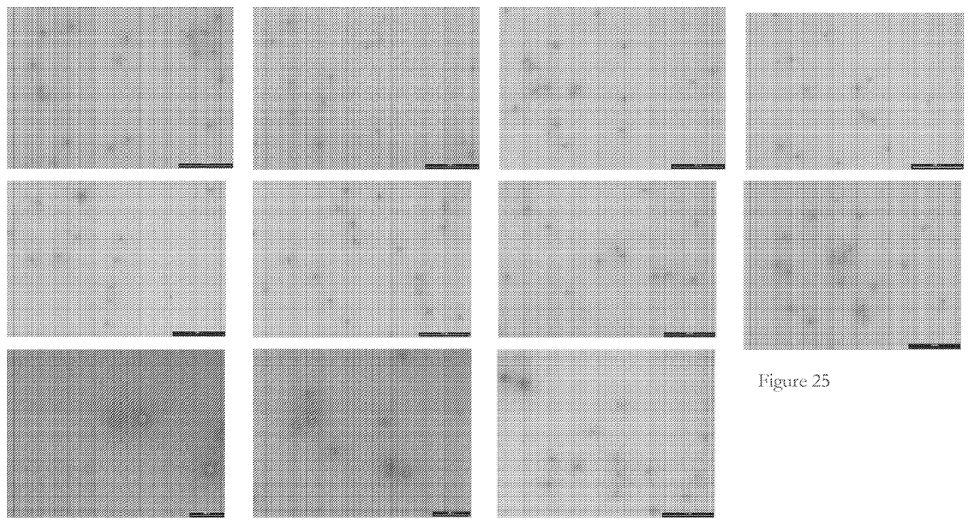

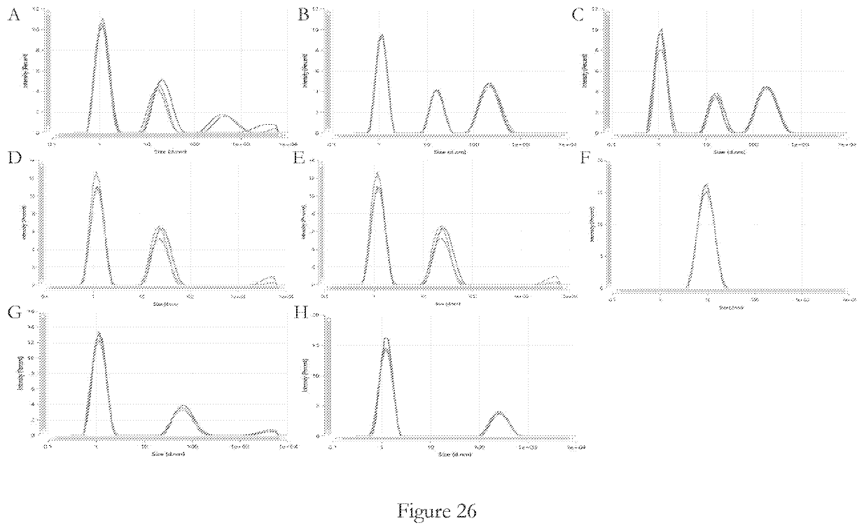

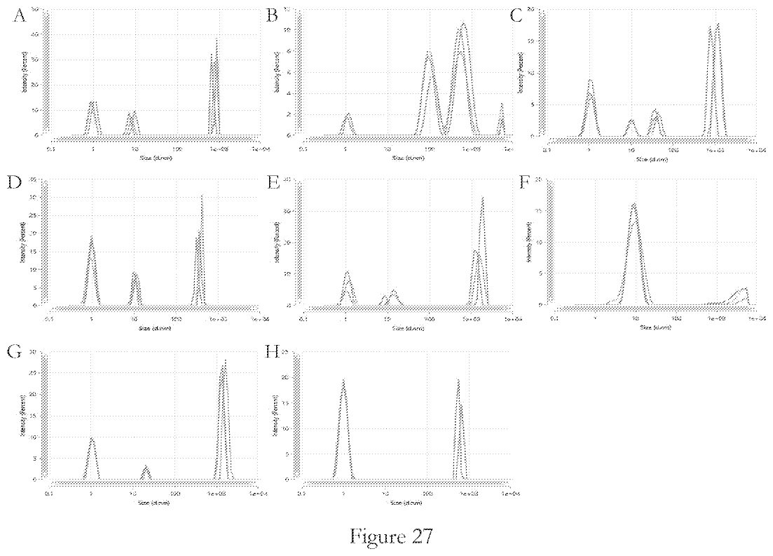

Transmission electron microscopy was used to determine the ratio of full and empty AAV2 capsids. The first capsid visualization utilized the standard protocol from the Nano Imaging Lab of the University of Basel and a sample with a titer of 1.0·10¹⁰ vg/mL. As shown in FIG. 3A), insufficient numbers of capsids were visualized. Such low amounts of capsids did not allow a quantification of full and empty capsids. Based on this result the sample preparation was modified and a higher titer of 1.0·10¹¹ vg/mL was used. This approach resulted in much higher numbers of stained capsids as shown in FIG. 3B). Full capsids appeared as white dots because the uranyl acetate cannot enter the capsids. However, empty capsids contain a dark spot in the middle of the capsid, caused by an entry of the dye into the capsids. Manually counting of all capsids on 11 pictures (see FIG. 25) enabled an estimate of the number of full and empty capsids. The sample contained 58.3% full and 41.6% empty capsids. Comparison of both experiments revealed that a titer of at least 1.0·10¹¹ vg/mL and a modified staining protocol with only one washing step is required to visualize enough capsids for quantification.

### 5.1.4 DLS and MADLS of AAV2 samples

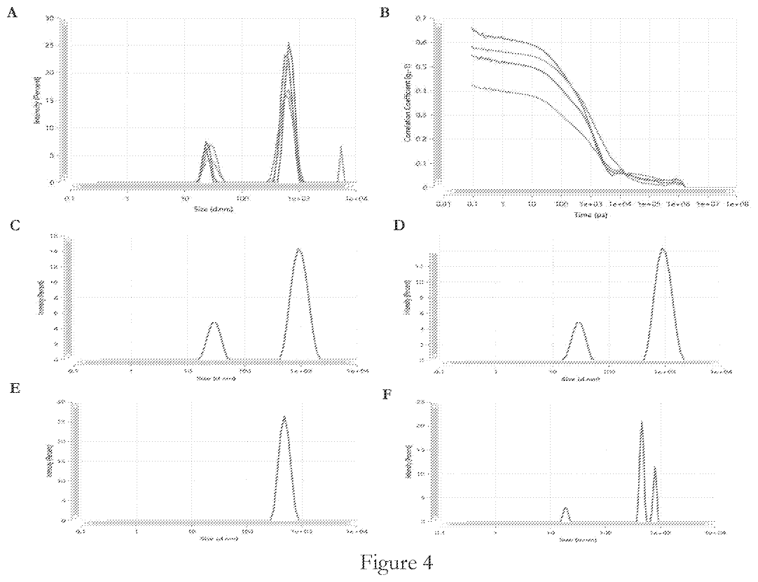

AAV2 has a size of 20-25 nm. Since DLS measured the hydrodynamic radius, a peak between 20-30 nm was expected. As shown in FIG. 4, back scatter measurements (4A) showed the presence of two particle species. One particle size distribution in the expected size range was obtained along with one much larger species around 640 nm (see appendix Table 7). Z-Averages of 643 nm confirmed the presence of a bigger particle species. Polydispersity index measurements also showed that the sample was polydisperse. As part of the method evaluation, an additional MADLS was performed to test if the results could also be obtained with this measurement. As shown in (4C), back-scatter is able to detect both particle species. Side-scatter (4D) confirmed a particle distribution obtained by the back scatter. However, the forward scatter (4E) only detected one peak at 500 nm. Back and side-scatter within a MADLS (4F) coincided, meanwhile, the forward scatter was not able to detect a particle species around 20-30 nm. Based on the ability to detect a particle species between 20-30 nm, a back-scatter was used during formulation studies to characterize AAV2 formulations.

### 5.1.5 Zeta Potential Measurements of AAV2

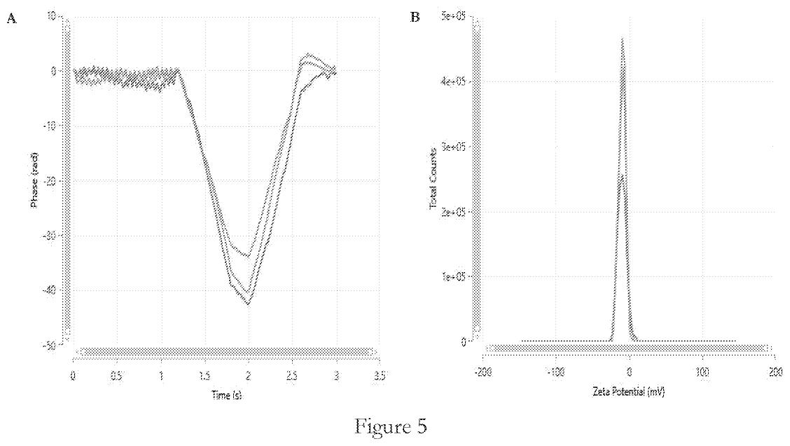

During the method evaluation, it was tested if it was possible to obtain a zeta potential when implementing the diffusion barrier method and using 130 μL AAV2 samples with a titer of 1.0·10¹¹ vg/mL. The phase plot measurements presented in FIG. 5A) as an indicator for the data quality, revealed a clear difference in the phase between the reference frequency and the measured beat frequency, as a function of time. This showed that it was possible to measure the zeta potential of AAV2 vectors when stored in PBS, pH 7.4 supplemented with 0.001% P188. The measurements performed during method evaluation were performed in triplicates and resulted in an average zeta potential of −9.46 mV.

5.1.6 U2OS-HTB-96 Cell Viability Assay with alamarBlue

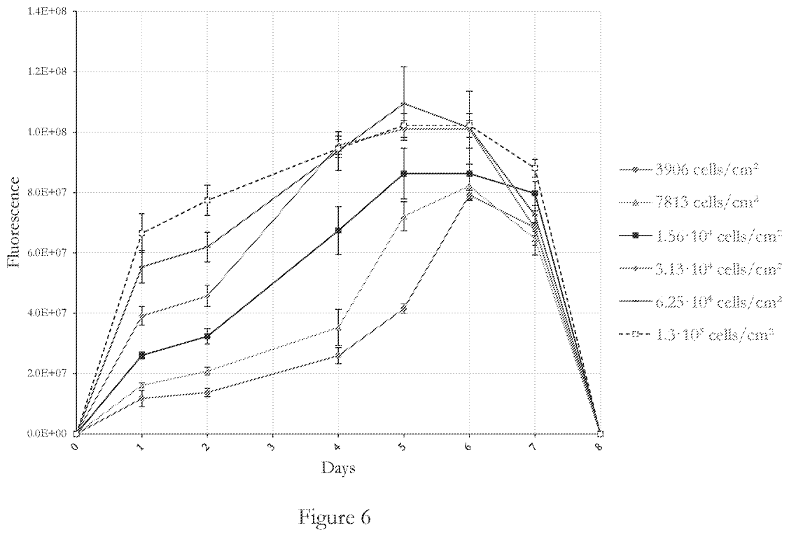

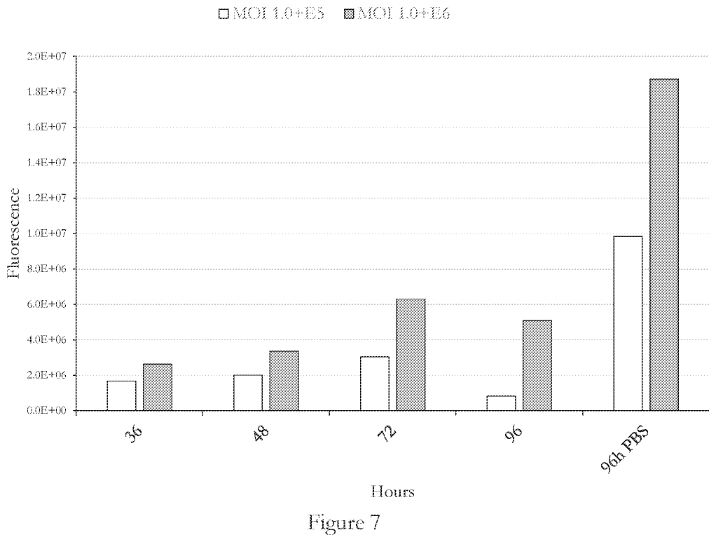

This viability assay tested how different seeding densities affect the grow rate and cell viabilities. As shown in FIG. 6, the assay showed that the growth rate is cell density dependent. A cell seeding density of 10 000 cells/well is highlighted in red, since this density later was used in development of the transgene assay. Fluorescence intensities showed that growth rates were dependent on the plated cell densities. Smaller seeding densities resulted in smaller initial fluorescence intensities. The highest initial density of 1.3·10⁵ cells/cm² reached a maximal cell density after four to five days. Smaller densities of 6.25·10⁴ cells/cm² and 3.13·10⁴ cells/cm² reached the same density after the same amount of time. This result illustrated that the growth rate with the highest seeding cell density is smaller compared to lower densities. Seeding densities of 7813 and 1.56·10⁴ cells/cm² reached the plateau after five days and the smallest seeding density reached maximal cell density after six days. Nevertheless, after reaching the maximal cell density, all wells recorded a decline in cell viability after 7 days. To be sure that the fluorescence is caused by the cells and not by components of the medium, the cells were killed after eight days and acted as a negative control. Addition of alamarBlue resulted in no fluorescence, showing that viable cells are needed to achieve a fluorescence signal.

### 5.1.7 Development of U2OS Cell Based GFP Expression Assay

Results of the first developed transgene expression assay are presented in FIG. 7. Illustrated fluorescence intensities have already subtracted the blanks containing only cells, medium and PBS. As shown in FIG. 7, results showed that both MOIs already caused a detectable fluorescence signal after 36 h. Fluorescence signals increase over time and reached their maximum after 72 h. Signal intensity was not only time dependent but also MOI dependent. During the course of this Disclosure, the term MOI is always used as the number of viral genomes that are added to one cell. An MOI of 10⁶ vg/cell resulted in higher fluorescence than an MOI of 10⁵ vg/cell. After 72 h the fluorescence caused by an MOI of 10⁶ vg/cell was twice as high as the fluorescence caused by an MOI of 10⁵ vg/cell. After 96 h the fluorescence decreased. Replacement of the medium with PBS at 96 h caused an amplification of the fluorescence signal. Compared to the fluorescence intensity at 96 h with medium, the fluorescence with PBS of the well incubated with a MOI of 10⁶ vg/cell increased almost four times in contrast to the well incubated with a MOI of 10⁵ vg/cell, which increased almost 12 times. Based on that result, fluorescence measurements for determining GFP expression were measured in PBS form then on.

### 5.1.7.1 Wavelength Scan of Recombinant GFP and MOI Titration

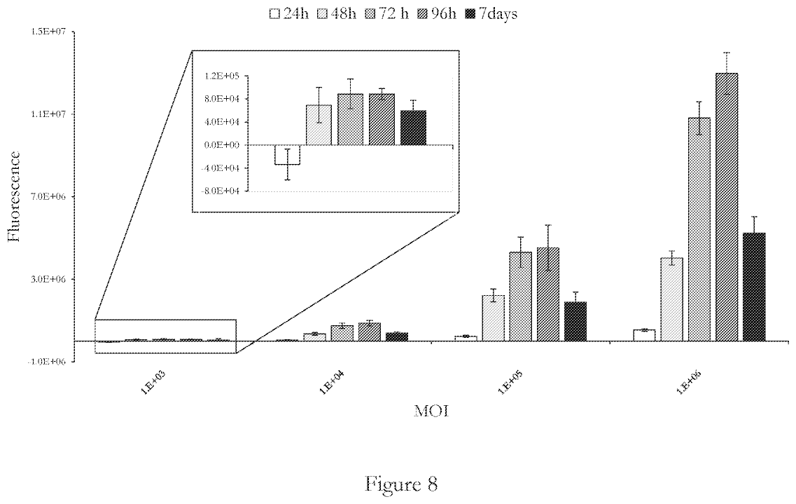

A rGFP wavelength scan revealed a maximal fluorescence intensity of rGFP when using an excitation wavelength of 460 nm and an emission wavelength of 515 nm. Afterwards, a titration of different MOIs was performed to find out if the plate reader can distinguish expressions caused by different MOIs and where the limit of detection is located. FIG. 8 illustrates the results of the MOI screen. It clearly shows a MOI dependent fluorescence intensity of transduced U2OS cells. The higher the MOI, the higher the expression. Moreover, the GFP expression increased over time. The biggest fluorescence increase appeared between 48 h and 72 h. Meanwhile, only a marginal fluorescence increase was seen between 72 and 96 h. Fluorescence readouts after seven days revealed a significant drop in expression. A MOI of 10³ vg/cell resulted in measurable fluorescence intensities after 48 h but did not result in significant expression changes. Based on these results the transgene expression assays during formulation studies were performed with a MOI of 10⁵ vg/cell and readouts after 72 h were used for comparison.

### 5.2 Formulation Study 1

### 5.2.1 AAV2 Titer Determination of Formulations at T0

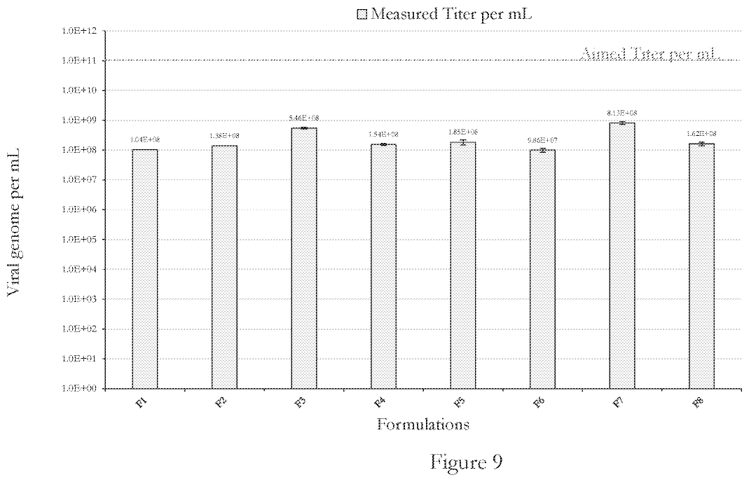

In the first formulation study, a T0 analysis was performed consisting of an AAV2 titration with qPCR as well as a GFP transgene expression assay. During compounding, titers of 1·10¹¹ vg/mL were aimed for. qPCR AAV2 titration results are illustrated in FIG. 9 and revealed that all eight formulations had much smaller titers than aimed for. Despite the variability of the titers between the formulations, all titers were in the range of 9.86·10⁷ vg/mL −8.13·10⁸ vg/mL.

### 5.2.2 GFP Expression of Formulations at T0

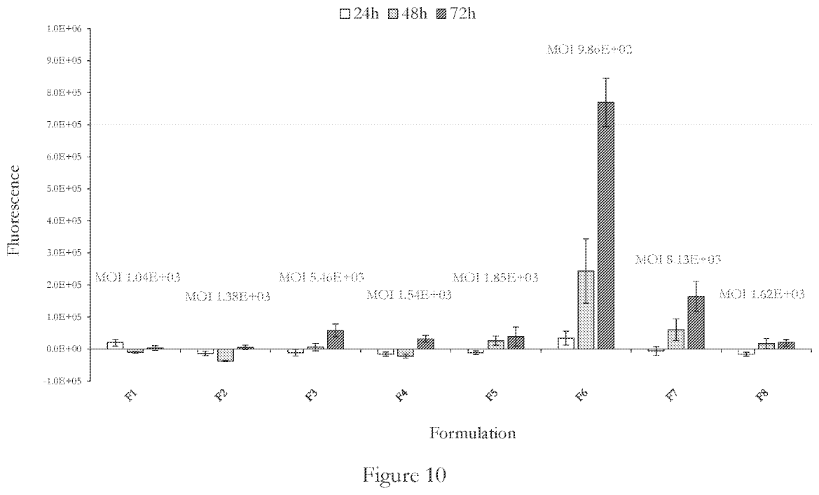

Data from the GFP transgene assay is summarized in FIG. 10. The displayed data has already subtracted the blank, which explains some of the negative fluorescence signals. These results illustrated that formulation 6 and 7 induced high GFP expressions. Meanwhile, all other formulations resulted in no measureable GFP expressions even after 72 h. Fluorescence intensities of Formulations 3, 4, 5, 8 reached only a fluorescence of 5.7·10⁵ or less. Formulations one and two showed no detectable fluorescence after that time. When adding the acquired data from the AAV2 titration, the results showed that GFP expression was done with a MOI of around 10³ vg/cell in all formulations. Although Formulation 6 caused much higher fluorescence intensities, the MOI was even smaller compared to the other formulations. Based on the low transgene expression and the low AAV2 titers, it was decided to terminate this study.

### 5.3 Results of Formulation Study 2

### 5.3.1 Freeze-Drying and Residual Moisture Content

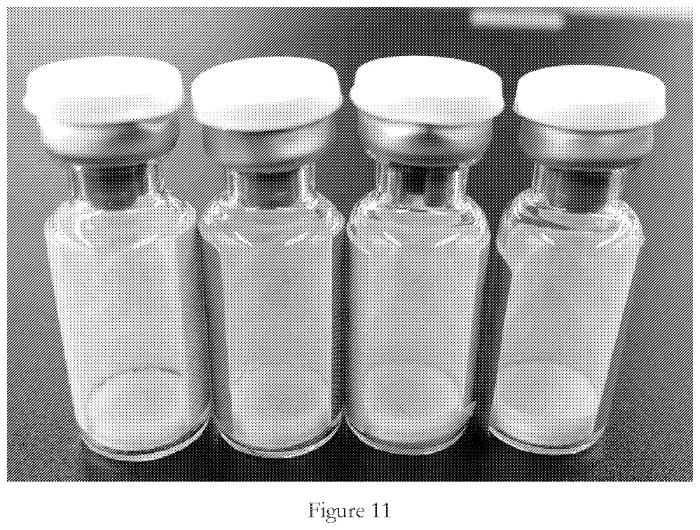

Visual inspection of lyophilisation cakes showed that five out of 32 cakes exhibit smaller cracks in the lyophilisation cake. A photo of the lyophilization cakes is shown in FIG. 11. Residual moisture content was determined with Karl Fisher Titration in three placebo vials. The analysis revealed a residual moisture content of 1.11, 1.13 and 1.23%.

### 5.3.2 Summary of all AAV2 Titer Determinations in Formulation Study 2

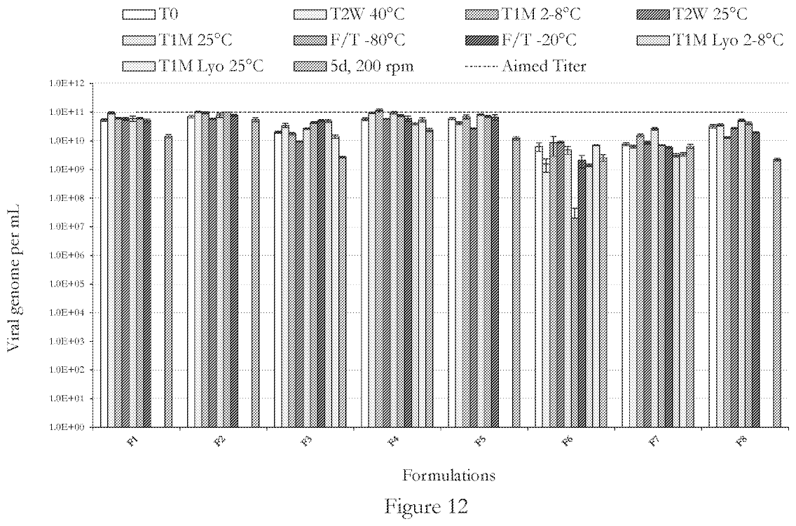

During each pull point, the titer of all formulations was determined using qPCR. The summary of all qPCR results is illustrated in FIG. 12. During compounding, all formulations were aimed for a titer of 1·10¹¹ vg/mL. Quantification showed that formulations 1, 2, 4 and 5 have low titer variabilities between different pull point measurements and they almost reached the aimed titer of 1·10¹¹ vg/mL. Exposure to agitation stress resulted in a titer decline in Formulations 1, 3, 5 and 8. Large titer variabilities between pull point measurements and large standard deviations of most measurement were seen in Formulation 6. Exposure of formulation 6 to −80° C. freeze-thaw cycles resulted in a titer decline of almost two logs. Likewise, all titers of formulation 6 were much smaller compared to the other formulations, and never exceeded 1·10¹⁰ vg/mL. Even though Formulation 7 showed no big variabilities between all measurements, most titer measurements underestimated the titer around one log. Noticeable is that formulation 2, 4, 6 and 7 have not experienced a titer decline after the agitation model. Besides the mentioned titer declines, formulations showed constant viral titers over the course of the formulation study.

### 5.3.3 Summary of All GFP-Expression Assays in Formulation Study 2

The following section presents the results from the cell based GFP transgene expression assay. Cells were transduced with eight different AAV2-CMV-GFP formulations. Upon successful transduction, the cells express GFP. By measuring the fluorescence with a plate reader, it was possible to directly quantify the GFP expression.

### 5.3.3.1 GFP Transgene Expression in U2OS Cells After Transduction with Formulations Stored at 2-8° C.

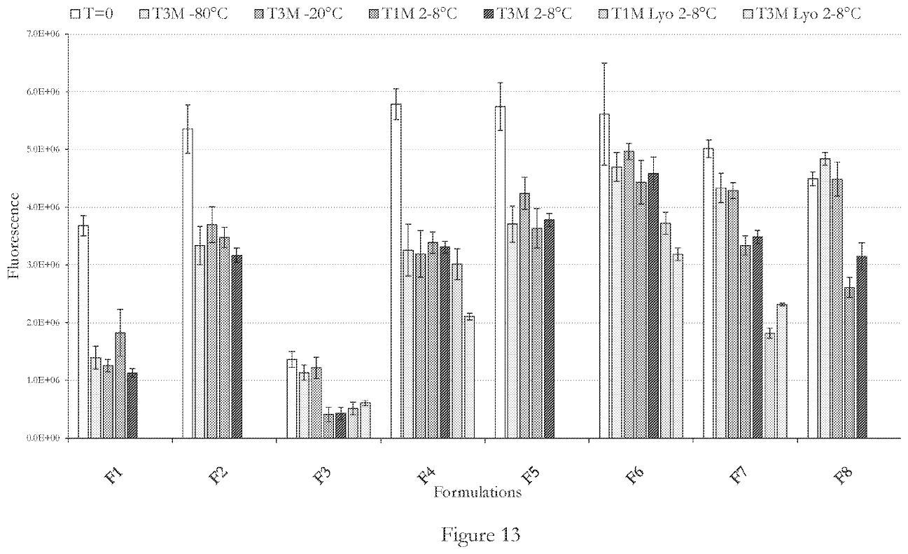

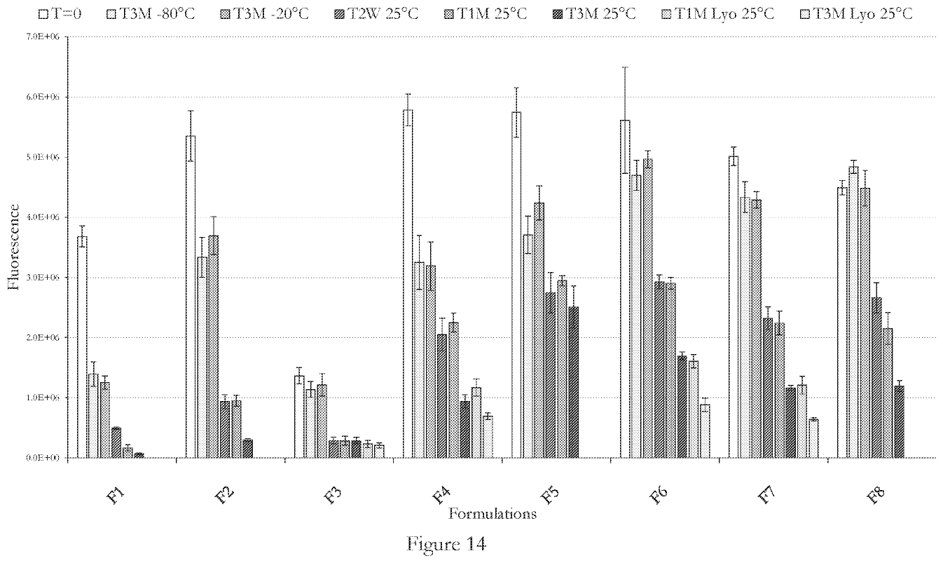

FIG. 13 displays the results obtained from the GFP transgene assay of all formulations exposed to 2-8° C. for different times. Each formulation provoked different fluorescence intensities at T0. Despite formulation specific GFP expression at T0, most formulations registered a significant fluorescence decline when frozen for three months independent of the freezing temperature. Formulations 6 and 8 showed no expression decline upon freezing and formulation 7 only recorded a small one. Exposure of formulation 8 to 2-8° C. for one and three months resulted in much smaller expression in contrast to the storage for three months at −20 or −80° C. Other formulations stored at 2-8° C. illustrated similar expressions compared to the frozen ones. No difference in expression between one and three months of storage were seen when stored at 2-8° C. Freeze-drying of formulation 3 and 4 followed by a storage at 2-8° C. for one month showed similar expressions in contrast to the liquid formulations. Whereas formulation 6 and 7 resulted in significantly smaller fluorescence intensities upon freeze-drying. During all pull points, transduction with formulation 3 resulted in much smaller absolute fluorescence intensities compared to the other formulations. Formulation 1 revealed smaller expressions compared to formulation 2 during the whole study already at T0.

Absolute GFP expressions during all pull points were the highest in formulation 6. MOIs applied in GFP expression assays (see appendix Table 9).

### 5.3.3.2 GFP Transgene Expression in U2OS Cells After Transduction with Formulations Stored at 25° C.

The results in FIG. 14 show that the GFP expression significantly declined after storage for two weeks at 25° C. Despite a big fluorescence decline after storage for two weeks at 25° C., no further declines were observed when stored for one month. But after three months of storage at 25° C., expression further dropped in all formulations. Formulations 1 and 2 displayed the biggest decline in expression after storage at 25° C. for two and four weeks. Again, formulation 6 resulted in the highest absolute GFP expression. All freeze-dried formulations except formulation 3 resulted in even smaller GFP expressions upon transduction than formulations, which were not freeze-dried. Freeze-drying followed by a storage of three months at 25° C. resulted in even lower expressions compared to storage for one month.

### 5.3.3.3 GFP Transgene Expression After Exposure to 40° C., Freeze-Thaw Cycles and Agitation Stress

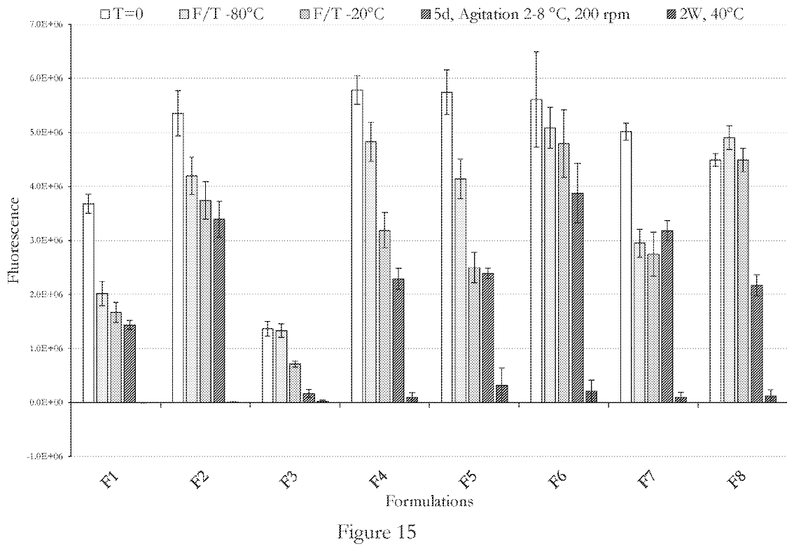

FIG. 15 illustrates GFP expressions after formulation were exposed to 40° C. or stress conditions such as freeze-thaw and agitation stress. Formulations 6 and 8 did not record any GFP expression declines after freeze-thaw cycles to −20° C. nor −80° C. All other formulations showed a decline of expression upon freeze-thawing. Cycles to −20° C. caused more distinct declines in expressions in formulations 3, 4 and 5 in contrast to cycles to −80° C. All other formulations revealed similar expressions compared to −80° C. cycles. Presented data showed that horizontal shaking for five days at 2-8° C. caused even lower GFP expressions than the freeze-thaw cycles. No formulation was resistant to shaking stress but formulation 6 showed the smallest expression decline compared to the other formulations. In formulations 1, 2, 5 and 7, agitation stress caused a similar decline in GFP expression as the exposure to −20° C. freeze-thaw cycles. Other formulations resulted in lower expressions. GFP expression in formulation 8 was significantly reduced after shaking for five days. Exposure to 40° C. for two weeks prevented any GFP expression in all formulations.

### 5.3.4 Summary of All Z-Averages and Particle Size Distributions Measured with DLS in Formulation Study 2

FIG. 17 (A) illustrates the Z-Averages of all formulations during all measured pull points. For formulations not showing any aggregation Z-Averages between 20-30 nm were expected. Data shows that formulation 6 has the smallest Z-Averages during all pull points not exceeding 31 nm in contrast, all other formulations had significantly higher Z-Averages already at T0. Formulation 1 showed highest Z-Averages around 1500 nm already at T0. Meanwhile Formulations 2, 3 had smaller Z-Averages around 400 nm at T0. Additionally, formulations 2, 3 and 7 showed low Z-Averages compared to the other formulations. Upon exposure to 25° C. for either two weeks or one month resulted in significant Z-Average increases in several formulations. Formulations 1, 4, 5, 7 and eight recorded such increases. Formulations 2 and 3 have not shown any Z-Average increase upon exposure to 25° C. for two weeks or one month. Highest Z-Averages after exposure to agitation stress were seen in formulation 8. This formulation not only showed the highest Z-Averages after shaking, it also had the highest values after freeze-thaw cycles to −20° C.

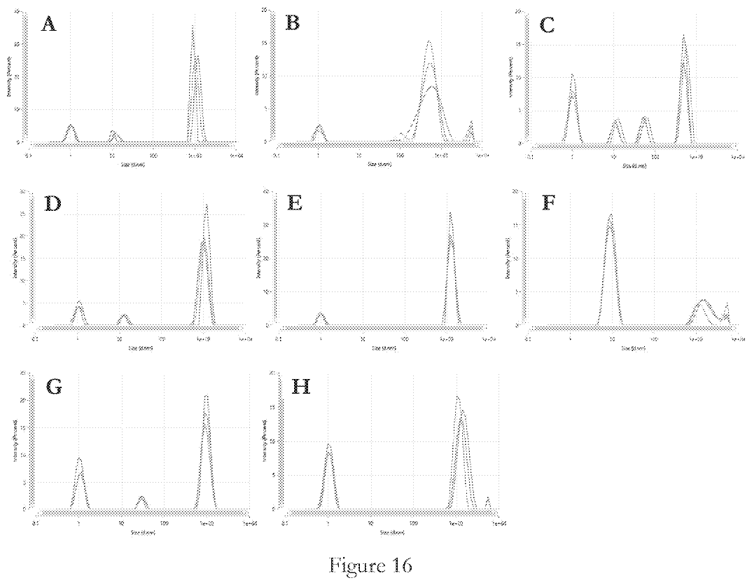

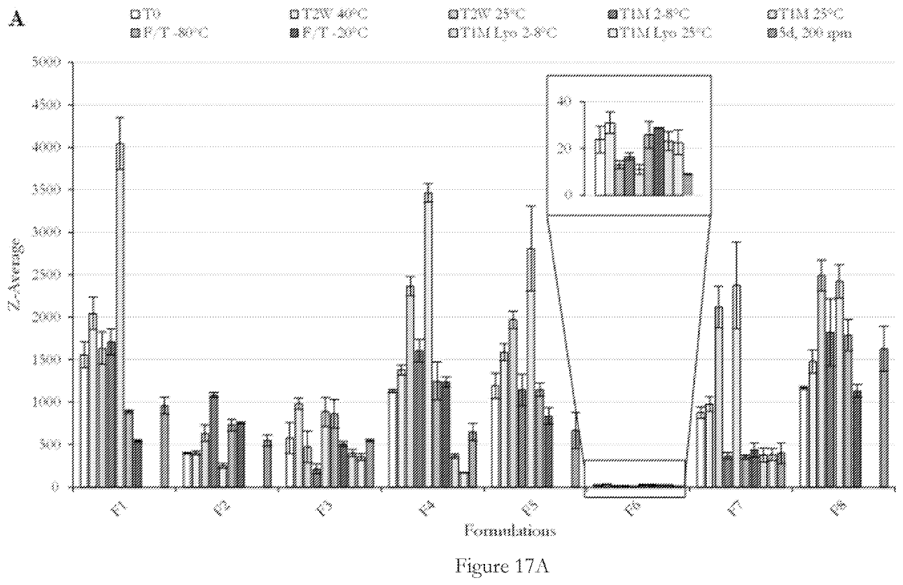

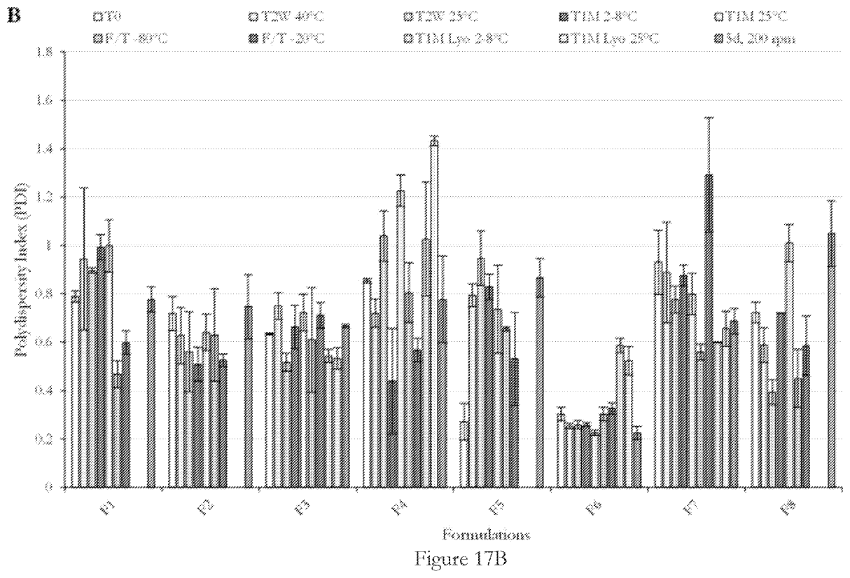

When looking at the particle distribution of each formulation at T0 displayed in FIG. 16, only formulation 7 shows a small particle size distribution at 30 nm.

All other formulations did not show any size distributions in that range. Formulation 6 containing 1% albumin had a very prominent particle size distribution around 10 nm. Formulations 1, 2, 3, 4 and 5, which contained 0.02% PS80 also revealed a peak at 10 nm. All formulations showed a specific, particle size distribution at higher nanometer ranges as already detected during the method evaluation experiments. In formulation 1 this peak was around 1000 nm, meanwhile in formulation 2 it was at 566 nm. Similarly, formulation 4 showed a size distribution at 1074 nm and formulation 3 at 522nm. Formulation 5 and 6 revealed a peak at 1292 nm and 1561 nm, respectively. In formulations 7 and 8 a particle size distribution was found at 957 nm and 1320 nm, respectively. During the course of this study, particle size distribution profiles described at T0 stayed constant. Especially peaks described around 1, 10, 30 nm stayed the same in the respective formulations at all conditions and pull points. Only the peaks described at higher nanometer ranges recorded some shifts during the course of the study. In the case of formulation 2, distributions which were barley seen around 100 nm at T0 gained intensity after exposure to 25° C. for four weeks. DLS was also performed with placebos at T0. No peak shift were observed during storage. Z-Averages of placebos were between 3.2 and 8.5 nm in all formulations. All placebos except placebo 6 containing 1% albumin showed a peak at 1 nm. Placebos have not shown any peaks at high nanometer ranges in contrast to the formulations. Size distributions of formulations exposed to 25° C. for one month and placebos at T0 are shown in FIGS. 29 and 30.

Polydispersity measurements illustrated in FIG. 17 (B). According to the literature, a monodisperse AAV formulation has PDI values around 0.2 or smaller. Thus, formulation 6 was monodisperse and became polydisperse when freeze-dried and stored at 2-8° C. as well as at 25° C. At T0 also formulation five revealed monodispersity, but became polydisperse upon storage. All other formulations were polydisperse from T0 and stayed polydisperse. Formulation 8 had not only the highest Z-Averages upon shaking, also the polydispersity was higher compared to the other formulations.

### 5.3.5 Summary of Sub-Visible Particle Measurements in Formulation Study 2

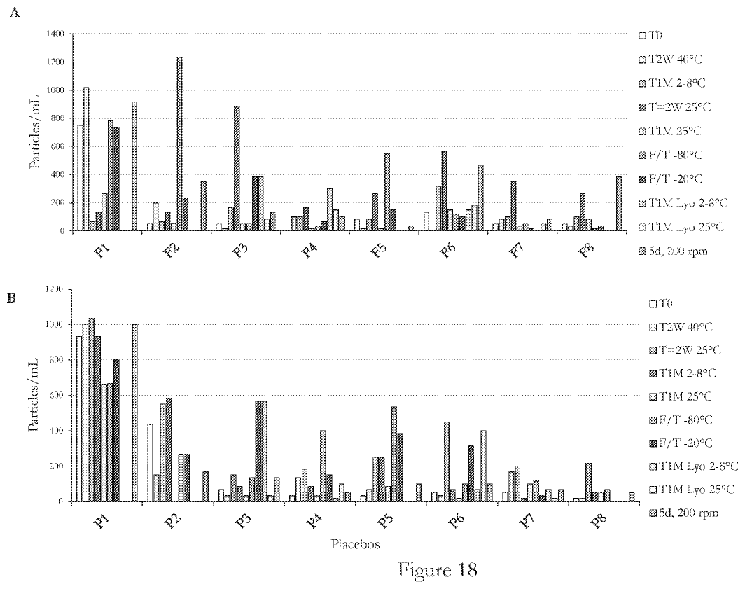

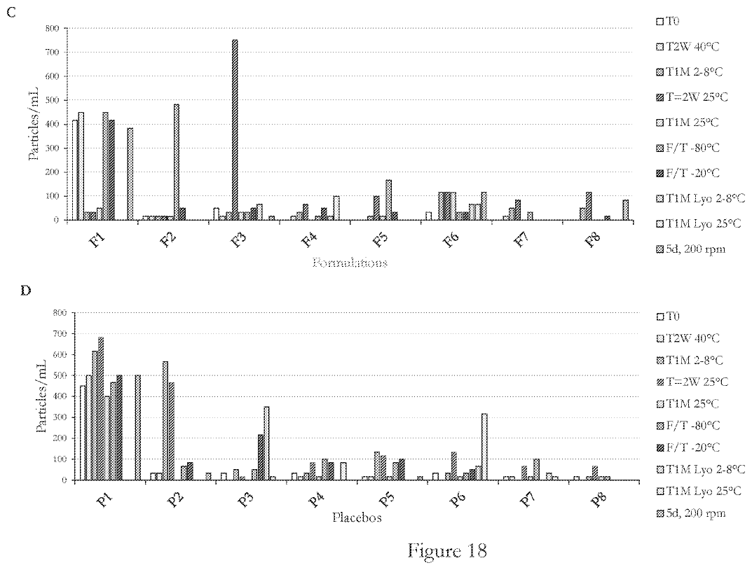

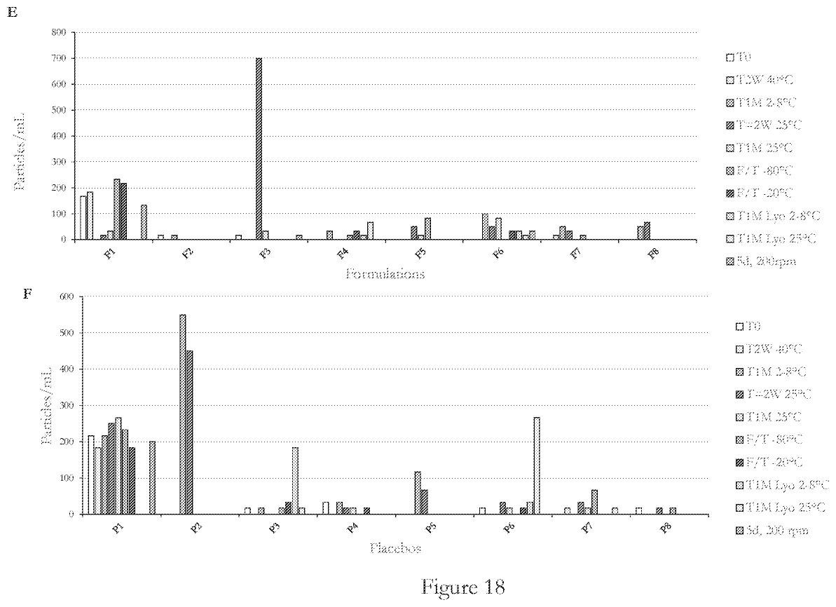

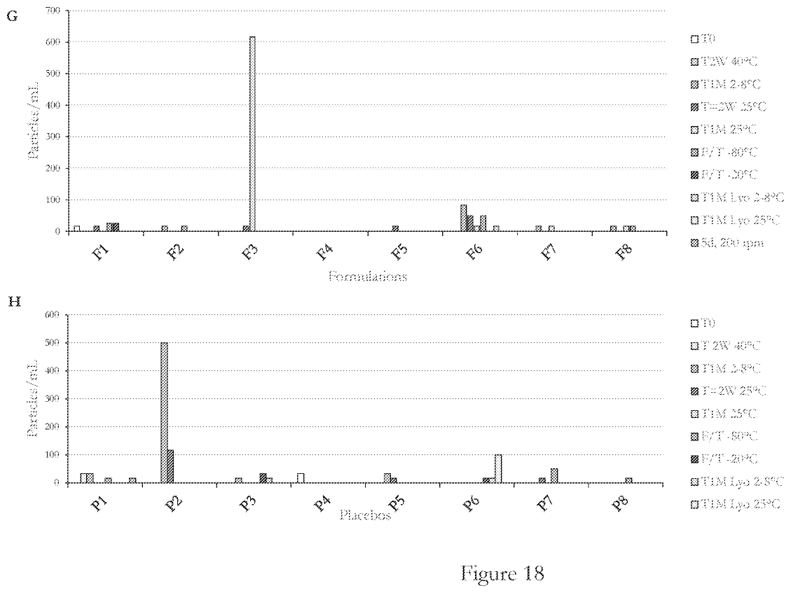

Since all samples were diluted for determining sub-visible particles, the received particle counts were multiplied with the dilution factor. After counting, the software divided the particles in four groups according to their size. Measurements revealed very low particle counts in all formulations. All counts were compared to respective placebos. As Shown in FIGS. 18B, 18D, 18F and 18H many Placebo measurements showed higher particle counts in contrast to the particle counts in the formulations. Placebo 1 showed the highest particle counts over all time points compared to the other placebos. Meanwhile Formulation 1 had only higher particle counts at T0, after storage at 40° C., freeze-thaw cycles and after agitation stress compared to other formulations. Upon exposure to −80° C. freeze-thaw cycles, Formulation 2 had significantly higher 2, 5 and 10 μm particle counts in contrast to placebos. Similarly, formulation 3 had higher 2, 5, 10 and 25 μm particle counts when exposed to 25° C. for two weeks. All other formulations had no significant bigger particle counts compared to their placebos. Most sub visible particles were detected in formulation and placebo 1. See FIGS. 18-21.

### 5.3.6 Summary of Zeta Potential Measurements in Formulation Study 2

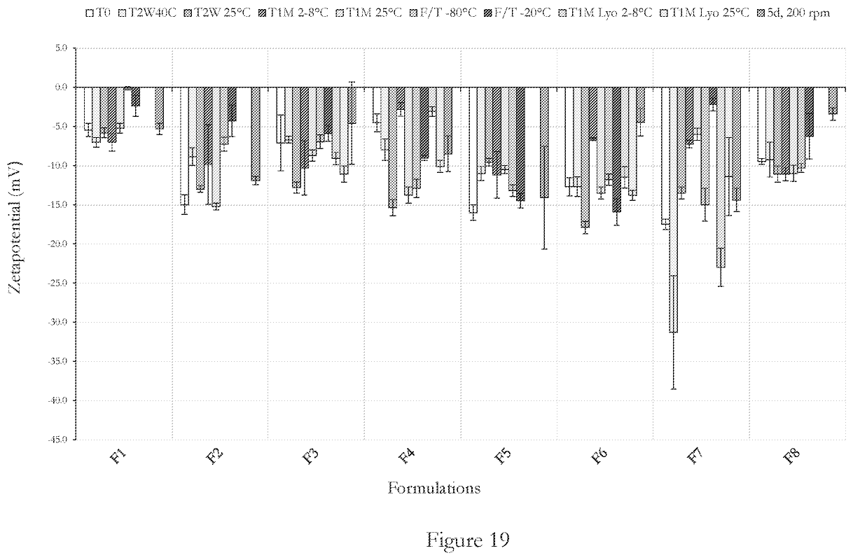

FIG. 19 shows zeta potential of all formulations. All formulations have a negative zeta potential. Big variabilities between the pull points were observed, especially in formulation 7. Zeta potentials of formulations 1 and 8 showed smaller variabilities between the pull point analyses. Formulation 1 had the smallest zeta potentials during all pull points. In formulation 7 exposure to 40° C. for two weeks as well as freeze-drying and storage at 2-8° C. resulted in high zeta potentials of around −31 mV, meanwhile ten freeze-thaw cycles to −20° C. resulted in a zeta potential of −2 mV. Exposure to agitation stress resulted only in formulation 6 to an increase in zeta potential. As already shown during method evaluation, the zeta potential of formulation 8 at T0 amounted to −9.46 mV which aligns with the AAV2 surface potential described in the literature. Zeta potential after agitation was the smallest in formulation 8.

### 5.3.7 Summary of pH Measurements in Formulation Study 2

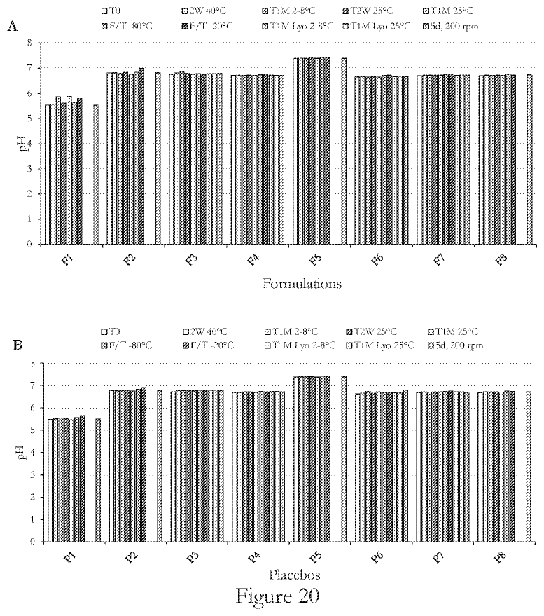

Most formulations showed constant pH values over the course of the formulation stability study. A small pH increase was observed in Formulations 1 and 2 as well as Placebo 1 and 2 after −20° C. freeze-thaw cycles. Similarly, formulation 1 showed a pH increase from 5.5 to 5.8 after storage for one month at 2-8° C. and 25° C. See FIGS. 20(A) and 20(B).

### 5.4 Additional Results

### 5.4.1 Filtration of Formulations Containing 150 mM Sodium Chloride Compared to Formulations Without Sodium Chloride

Based on results obtained from the first formulation study, additional experiments were conducted to investigate the reasons for the titer loss after sterile filtration. These experiments were conducted between pull point analysis of formulation study 2. FIG. 21 illustrates the impact of sodium chloride on the filtration ability of AAV2 formulations. Formulations supplemented with 150 mM sodium chloride showed a complete titer recovery after sterile filtration. Insignificant amounts of viral genomes were lost in the sterile filtration process. Whereas formulations not containing any sodium chloride registered a titer loss of more than 1.5 Log units in all three filters. Filtration through the Puradisc PVDF filter resulted to a slightly smaller titer of 2.27·10⁹ vg/mL compared to the Millex and Anotop filter resulting in titers of 3.87·10⁹ vg/mL and 3.53·10⁹ vg/mL, respectively.

### 5.4.2 Impact of 150 mM Sodium Chloride on the Particle Size Distribution Measured with DLS

TABLE 6
DLS raw data of formulations supplemented with 150 mM NaCl
compared to formulations without NaCl. sterile filtration was
performed using a 13 mm 0.2 μm Millex PVDF syringe filter

| | No Sterile Filtration | | Sterile Filtration |
|---|---|---|---|
| | 20 mM | 20 mM | 20 mM |
| | L-Histidine, | L-Histidine, | L-Histidine, |
| | 290 mM | 290 mM | 290 mM |
| | Sucrose, | Sucrose, 150 | Sucrose, 150 |
| | 0.02% PS80, | mM NaCl 0.02% | mM NaCl 0.02% |
| Formulation | pH 6.8 | PS80, pH 6.8 | PS80, pH 6.8 |
| Z-Average | 586.7 | 82.98 | 27.36 |
| Polydispersity | 0.7623 | 1.315 | 1.27 |
| Peak 1 | 1.125 | 1.16 | 1.19 |
| Peak 2 | 11.25 | 26.11 | 25.64 |
| Peak 3 | 100.3 | 458.60 | 272.90 |
| Peak 4 | 782.3 | — | — |

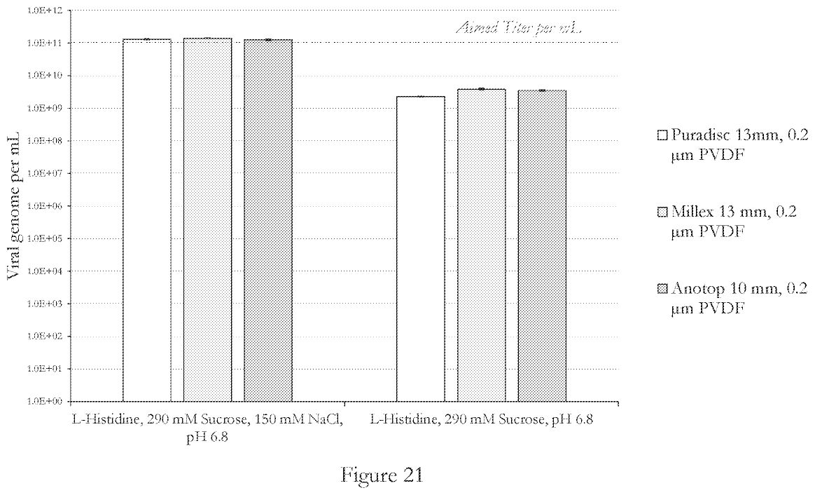

As shown in FIG. 22 and in Table 6 the particle size distribution differs between the formulations containing 150 mM sodium chloride compared to formulations without any salts. Both formulations consisted out of the same buffer, same excipient and sucrose concentrations. Results showed that the Z-Average of the formulation without any NaCl is 587 nm. Supplementing sodium chloride to the formulation lowered the Z-Average to 83 nm. Z-Average further drops when formulations were sterile filtered through a 0.2 μm Millex PVDF filter. Polydispersity was almost twice as big in formulations containing NaCl compared to formulations without NaCl. Formulations supplemented with 150 mM NaCl revealed a size distribution around 20-30 nm meanwhile formulations without NaCl did not show any particle size distribution in that range. Instead particle species around 780 nm was detected. Addition of NaCl profoundly changed this size distribution profile. Sterile filtered formulations showed much smaller standard deviations compare to not sterile filtered samples.

### 5.4.3 Thermal Shift Assay with SYPRO-Orange and SYBR-Gold

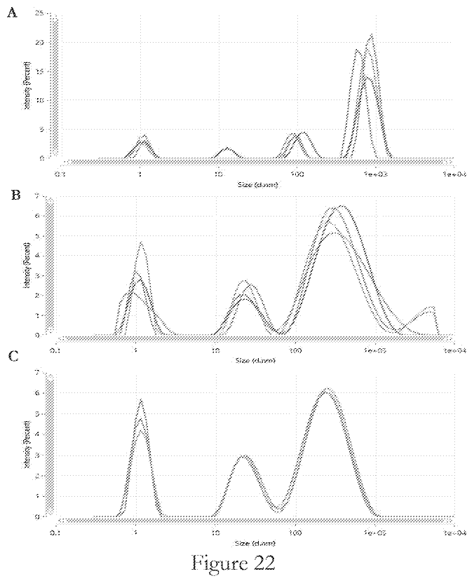

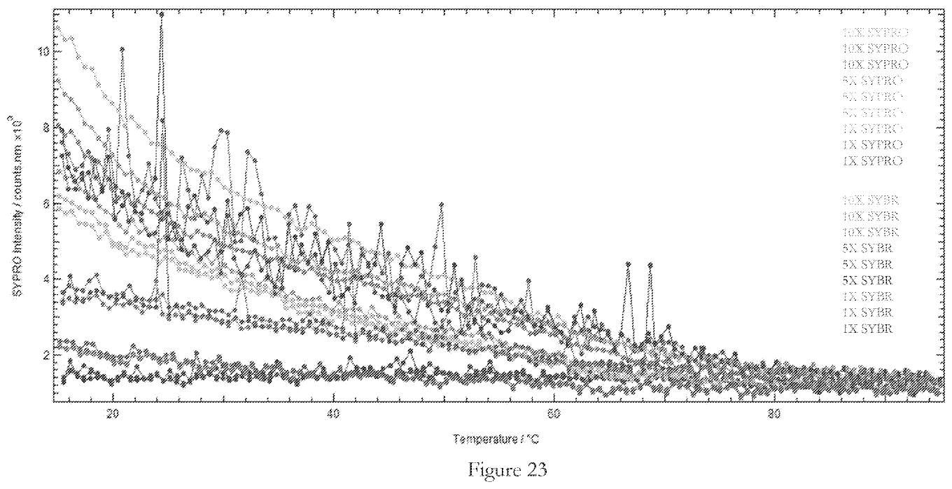

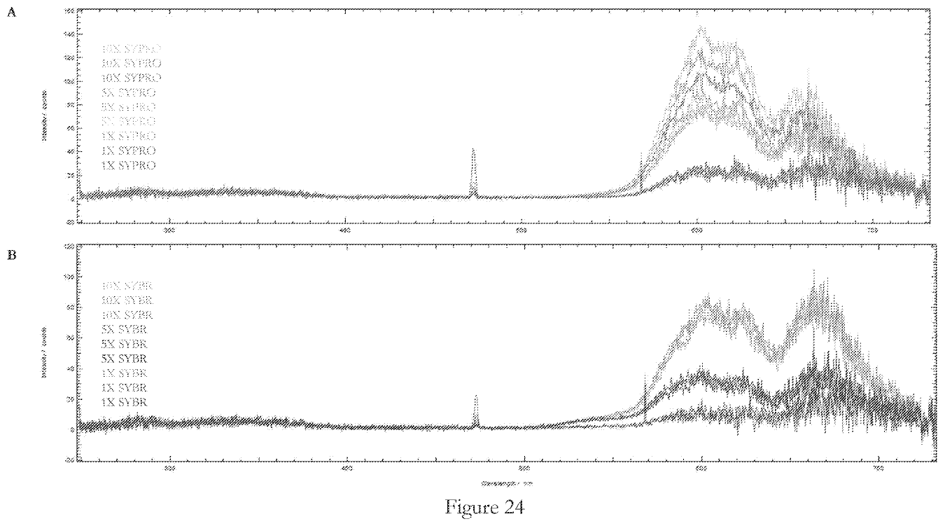

As described in the introduction, DSF is an attractive tool for studying the impact of excipients, buffer systems and pH ranges on the thermal stability of capsids. Method evaluation was conducted between pull point analysis of formulation study 2. Differential scanning fluorimetry was performed with samples having a titer of 1.0·10¹¹ vg/mL. Since these titers correspond to very low concentrations, SYPRO-Orange and SYBR-Gold were added to enhance the signal intensity. The aim was to observe the thermal shift caused by capsid denaturation and determine the melting temperatures. Results in FIG. 23 showed that no thermal shift was observed in any samples. Even though no thermal shift can be seen, FIG. 24 (A) and (B) show that excitation of dye containing samples cause detectable and concentration dependent fluorescence signals. No further measurements with higher AAV2 concentrations were performed.

### 6 Discussion

### 6.1 Evaluation and Development of Analytical Methods for Characterization of AAV2

As expected DNase I sample treatment reduced titers, because residual host cell impurities are present in the samples. Without this digestion step, the titer will be overestimated because host cell DNA interferes with viral titer quantification. A similar titer decline upon DNase I treatment with two units of DNase I was observed and described by Dobnik and colleagues [70]. Presence of such impurities not only highlighted the need of a DNase I digestion step, it was also a red flag for potential aggregation. As Wright and colleagues has proposed, residual host cell DNA can mediate aggregation although concentrations are pretty low [30].

Thermal capsid opening increased AAV2 titers and indicated that short capsid opening time at the beginning of the qPCR cycle is not sufficient for releasing all viral genomes before analysis. Combination of both treatments enabled a precise determination of the titer given by the supplier.

In the Eppendorf adhesion experiment, it was shown that AAV2 titers increase when samples are mixed after 24 h of storage in Eppendorf tubes. However, one cannot conclude whether the increase is caused by detaching AAVs from the tube walls or by homogenizing samples. Nevertheless, this knowledge was applied and samples stored in Eppendorf tubes were always mixed by pipetting up and down for at least ten times with half of its volume.

For TEM measurements, it was shown that a modification of standard negative staining protocol and an increased titer were needed in order to visualize enough capsids on the copper grid. It is plausible that a higher titer results in higher numbers of AAV capsids on the grid, however literature showed that already a titer of 10⁹-10¹⁰ vg/mL resulted in a high amount of capsids on the grid. Therefore, it can be assumed that major modifications in sample preparation resulted in the increased capsid numbers. Especially the prolonged incubation time of the vector suspension on the copper grid enabled that more capsids adhere and thus could not be washed away during the following washing steps. Results showed that the sample contained slightly more full capsids than empty capsids. This is an indicator that the AAV2 supplier extensively purified the AAVs since not purified AAV samples normally have much more empty capsids.

It was shown that zeta potential measurements can be performed with the diffusion barrier method and titers of 1·10¹¹ vg/mL were sufficient to obtain a readout. Results of the zeta potential method evaluation showed an AAV2 surface potential of −9.46 mV at pH 7.5 which correspond to the literature value for AAV2 measured at a pH of 7.5.

During developing GFP expression assay, it was shown that a concentration dependent GFP fluorescence can be measured with a plate reader. Fluorescence intensities increased when the medium in the well was replaced with PBS before fluorescence measurements. An explanation for this result is that the medium containing phenol red acts as a quencher for GFP. Additional interferences by phenol red can be caused when cell culture pH are shifting and thus the phenol red absorbance spectrum changes. To prevent such confounding factors, it was decided to replace the culture medium with PBS during the measurement to gain signal intensity on the one side and on the other side to guarantee consistent results over time.

First expression experiments were performed with excitation wavelengths of 488 nm and emission wavelengths of 520 nm which were described in the literature. Since signal intensities were quite low it was decided to perform a wavelength scan with an rGFP control. Using this control, the ideal excitation and emission wavelengths were identified (namely 460 nm and 515 nm, respectively). Use of these wavelengths further increased signal intensities.

In the next step, it was shown that the plate reader was able to distinguish the GFP expression caused by different MOIs. As expected, this result highlighted a concentration dependent expression. Likewise, the GFP expression was already detected after 24 h but further increased over time. Such a fast onset of fluorescence showed on the one hand that a human cytomegalo virus (CMV) promotor is an efficient and potent promotor suited for monitoring GFP expression in U2OS cells and on the other hand that AAV transduction occurred within a few hours from incubation with cells. Findings were supported by Bartlett and colleagues, who showed that AAV infection and genome transduction into the nucleus occurs within three to four hours [39].

Maximal expressions were reached after three to four days in all MOIs. With looking at the cell viability assay it becomes apparent that this correlates with the cell density and cell viability. After four days highest cell densities were observed in the well plate indicating that the cells could not further increase their density. Cell density stayed constant for two more days, showing that cells were in an equilibrium. After seven days, the cell viability significantly declined which could also be seen in the GFP expression assay. A GFP expression and thus a fluorescence decline after seven days was seen because GFP expressing cells have died. Reason for a loss of cell viability is a too high cell density within the well and a lack of enough nutrients to maintain the cell homeostasis.

Even though an MOI of 10³ vg/cell resulted in a measurable GFP expression, no changes in expression were detected over time. Therefore, this MOI was defined as the lowest limit of detection. A higher MOI of 10⁴ vg/cell already resulted in a time dependent GFP expression. Based on these results, it was decided to conduct the formulation study with an MOI of 10⁵ vg/cell. Such an MOI resulted in strong GFP expression and fluorescence. Likewise it was possible to monitor a decline in expression without directly reaching the lower limit of detection. Another advantage compared to higher MOIs of 10⁶ is that it was possible to quantify GFP expression in five replicates instead of only one. The cell viability assay showed that this density enabled monitoring of the GFP expression over four days without losing cells and expression. Higher densities would have not guaranteed such long observations, which could bare the risk of insufficient expression times. Whereas lower densities might be too small to cause a measureable signal.

During the evaluation of DLS as a method to monitor AAV2 particle size distribution, it was shown that AAV2 capsids can be detected with a titer of 10¹¹ vg/mL. A simple back-scatter analysis was able to reveal a size distribution around 20-30 nm which was assigned to AAV2. Although DLS is able to detect AAV2, another peak appeared at around 600 nm. Later experiments have shown that aggregates caused these size distributions. Since signal intensities were quite low, the device manufacturer was contacted regarding the lower limit of detection for AAVs. According to the device supplier, the capsid amounts used were equal to the lower limit of detection. MADLS measurements were performed to confirm results obtained from backscatter measurements. Because forward scatters were less sensitive than back and side scatters, it was not able to detect a size distribution at the expected range between 20-30 nm. Thus, it was decided to only apply the back scatter measurements in the formulation studies.

As described in the introduction, thermal shift assays with DSF could add valuable data within a formulation study. Despite addition of dyes no thermal shifts were detected. Excitation of the dyes showed a concentration dependent signal intensity, this proofed, that enough dye was present in the samples. Missing thermal shifts are therefore created by too small AAV2 concentrations and not by insufficient dye concentrations. No further experiments were conducted because higher titers were required. From the literature, measurable thermal shifts when using titers around 6·10¹¹-1·10¹² vg/mL were expected [102].

### 6.2 Formulation Study 1

After conducting the qPCR expression assay at T0 in the formulation study 1, it became apparent that all formulations contained lower titers than aimed for. This finding was confirmed by the GFP expression assay. Even though titers were low, significant GFP expression was observed in the formulation containing albumin and hyaluronic acid. Although significant expression was seen, qPCR measurements showed similar titers in contrast to the other formulations. Based on that result, it was assumed that albumin as well as hyaluronic acid potentially interfere with the qPCR method and artificially lower the vector titer.

Titer loss observed in all formulations can be explained by AAV aggregation or adsorption to filters. DLS results during method evaluation potentially support the first hypothesis, since they revealed a Z-Average of 643 nm. Thus additional experiments in between the pull points of formulation study 2 were designed to elucidate reasons and solutions to that problem.

As shown in additional experiments, an AAV2 formulation could be sterile filtered when provided with 150 mM NaCl independently of the PVDF filter brand. According to the literature, such salt concentrations prevent AAV2 aggregation, however, adsorption cannot be completely ruled out [30].

With additional DLS measurements, it was shown that sodium chloride prevents aggregation and results in much lower Z-Averages. Looking more closely at the particle size distribution, it was shown that upon supplementation of 150 mM NaCl a particle distribution at 20-30 nm appeared, indicating that unaggregated AAV2 were present. This distribution was not recognized in the formulation not provided with any NaCl. Instead, such a formulation only revealed a peak at 782 nm indicating vector aggregation.

Sterile filtration of the formulation containing 150 mM NaCl resulted in lower standard deviations compared to measurements of not sterile filtered samples. Interestingly, a size distribution around 273 nm still appeared. Polydispersity Index confirmed that samples were polydisperse despite sterile filtration. This could be due to re-equilibration of AAV vectors after filtration to form aggregates.

It is worth mentioning that the presence of aggregates was observed by DLS in both cases (with and without NaCl). Only sterile filtration followed by an AAV2 titer quantification with qPCR was able to reliably show that aggregation was reduced and sterile filtration did not cause any loss of titer when formulations were provided with 150 mM NaCl.

As the formulation study was aiming at evaluating stability of formulations after lyophilisation, the selected formulations did not contain NaCl. This is because using such high level of NaCl would lead to a freezing-point depression, thus necessitating the development of an extremely long freeze-drying cycle. Meanwhile, albumin as well as hyaluronic acid were investigated as potential alternatives that might have the potential to prevent AAV aggregation and simultaneously can be freeze-dried.

GFP expression was observed in formulation 6 and 7 during formulation study 1. This finding demonstrated that less aggregation happened and more vectors passed through the sterile filter. Formulation with albumin showed more expression than the formulation with hyaluronic acid which most likely is caused by a more efficient prevention of aggregation. Nevertheless, GFP expression was lower compared to the expressions measured during method development, indicating that aggregation was not fully prevented.

### 6.3 Formulation Study 2

All formulation were successfully freeze-dried. Some lyophilisation cakes revealed minor cracks, which can be explained by cake shrinkage commonly observed in formulations containing sucrose and the small formulation volume used.

While the second formulation stability study showed generally higher titers in contrast to study 1, formulations containing albumin and hyaluronic acid demonstrated relatively lower titers, although their GFP expressions were high. This might point to a potential interference of both excipients with qPCR, a hypothesis that needs further investigation, since no literature described such a phenomenon. Except for one outliner outlier after F/T cycles to −80° C., titers stayed consistent during the course of the study.

It was shown that titers stay constant throughout the whole study, independently of their buffer and pH. Noticeable is that agitation stress as the only stress condition causes significant titer declines in several formulations. The hypothesis is that agitation stress broke some capsids and caused a release of their genomes. During the DNase I digestion step, this DNA is then digested and removed. Samples containing albumin, hyaluronic acid has not recorded a titer decline after shaking.

However, L-histidine and sodium phosphate with a pH of 6.8 were resistant against agitation stress. Several factors could be responsible. First it was concluded that the pH impacted the stability of the capsid, since a L-histidine pH 5.5 formulation recorded a titer decline compared to a L-histidine pH 6.8 formulation which was resistant to a titer decline. A higher pH of 7.5 also seemed to decrease stability against shaking stress. Therefore, a pH around 6.8 showed to be favorable concerning capsid stability.

Freeze-drying was successfully performed and did not result in any titer decline. Additionally, AAVs were freeze-dried without observing any loss in titer.

In contrast to titer measurements, transgene expression informs about the potency of a therapy. Even though titer stay mostly constant, expression is rapidly decreasing upon different temperatures and stresses. Exposure to different stresses showed that freeze-thaw cycles to −80° C. resulted in similar and in some cases in higher expressions compared to freeze-thaw cycles to −20° C. This finding contradicts Croyle and colleagues, who proposed that -80° C. freeze-thaw cycles more negatively impact transgene expression in contrast to −20° C. freeze-thaw cycles [27]. Addition of albumin or P188 to a phosphate buffer pH 6.8 resulted in resistance to freeze-thaw cycles of both temperatures compared to the GFP expression at T0. Albumin seemed to protect the capsids preventing any major capsid changes which result in impairment of transduction efficiency. Agitation stress and the resulting titer decline was also observed in the GFP expression. Especially formulation 8 containing P188 registered only half of its GFP expression when exposed to agitation stress in contrast to T0. It was concluded that P188 protects the capsids worse against agitation stress compared PS80. This finding was confirmed by titer measurements. Surprisingly, no expression difference was observed between formulation 4 and 5, although a titer difference was measured. Differences in expression after shaking between L-histidine pH 5.5 compared to pH 6.8 can be explained by the observed titer decline in the qPCR study. Most likely capsids at pH 5.5 are less stable and thus more susceptible to agitation and temperature stresses. Nevertheless, all formulations resulted in a loss of expression upon shaking, also formulations that registered no titer decline. Such results indicated that shaking stress either caused capsid breakage or lead to capsid changes resulting in decreased transduction efficiency of the vector.

L-Histidine or addition of albumin to a phosphate buffer performed well and showed only small GFP expression decreases in all mentioned stresses. Although melting temperatures of AAV2 are around 70° C., exposure to 40° C. is enough to extinguish any expression. It was assumed that exposures to such temperatures resulted in irreversible capsid changes and disabled the vector infectivity.

Throughout the whole study it was observed that the formulations containing sodium citrate observed much smaller GFP expressions in contrast to all other formulations already at T0. Citrate is known to act as a chelating agent. Whether this chelation capability has lead to the observed reduction in expression/potency remains to be evaluated.

An acidic pH of 5.5 resulted in much lower expressions already at T0 compared to formulations with higher pH. As described in the introduction, a lower pH results in the pore opening and externalization of the N-terminus of the VP1 protein. Such a conformational change allows an endosomal escape but might interfere with the receptor mediated uptake, impair capsid stability or later prevent a successful endosomal escape.

Freezing of all formulations either to −20° C. or −80° C. followed by a storage for three months resulted in a big decline of GFP expression in most formulations. Again, sodium phosphate buffer containing P188 or albumin were resistant to any expression decline and showed a similar expression as at T0. Both formulations already showed resistance to freeze-thaw cycles. Exhibition of such properties make sodium phosphate buffers supplemented with either P188 or albumin an excellent storage and shipping buffer. Such favorable characteristics explain why this P188 is frequently used by AAV manufacturers. Storage for three months at −20° C. and −80° C. was also highly successful in formulations containing albumin additionally it also stabilized expressions during storage at 2-8° C. Meanwhile P188 containing formulations recorded a significant expression decline when stored at 2-8° C. Its main usage should therefore be limited to long term storage at −20° C. or −80° C. For that reason the addition of albumin presents a new attractive alternative storage excipient superior to P188.

All other formulations showed a similar expression when frozen in contrast to storage at 2-8° C. for one or three months. No formulation showed changes in expression when stored at 2-8° C. for one month compared to three months. This result is valuable for the design of future stability studies. It particularly shows that instabilities resulting in reduction of GFP expression occurred before one month of storage at 2-8° C. Maximal decline was reached after one month of storage at 2-8° C. and did not further drop when stored for three months. Although several publications as well as recommendations of the AAV supplier advised not storing vectors at −20° C. no harmful impact was assessed on expression when stored at −20° C. compared to storage at −80° C. for three months. Impact of long term storage remains to be evaluated.

The results herein showed that a storage at 25° C. for only two weeks, resulted in much smaller GFP expressions in contrast to storage at 2-8° C. and no formulation was able to prevent a decline of expression. Interestingly, the expression was similar after four weeks compared to two weeks, but further declined when stored for three months. AAV2 were clearly instable at such temperatures.

A main goal of the study was to evaluate freeze-drying of AAV2 formulations. It was shown that freeze-dried formulations stored at 2-8° C. resulted in similar expressions as liquid formulations stored at 2-8° C. the only difference was that a further decline in expression was seen when stored for three months in contrast to the liquid formulations. Sodium hyaluronate as an excipients has the feature to elevate the glass transition temperature and is thus suitable for freeze-drying. Despite the favorable properties, formulations containing sodium hyaluronate showed lower GFP expression compared to other formulations. The results herein showed that liquid formulations presented similar or higher GFP expressions when stored at 2-8° C. or 25° C. for one or two months compared to freeze-dried formulations. The results contradict the results of Croyle and colleagues who showed that the infectious titer stayed constant for 90 days within freeze-dried samples [27].

Addition of albumin to AAV2 vector formulation resulted in the highest GFP expressions throughout the whole study, in contrast to other formulations. One reason could be that albumin binds to the capsid and forms an additional layer. With albumin bound to the capsid the virus uptake is additionally mediated by albumin specific receptors and is not only HSPG-receptor dependent [105]. Further investigations needed to be performed to investigate if albumin additionally protects the viral capsid from temperature and stress dependent structural changes.

Generally, GFP expression measurements underlined that expression is more impacted by the different formulations, excipients, exposed stresses and temperatures, compared to titers. It was shown that titer measurements are not reliable stability predictors. On the contrary, a titer decline upon agitation is associated with a GFP expression decline. Even though titer measurements enable minor predictions about the AAV2 stability and are crucial for dose estimations, they never should be solely used for stability predictions. They do not represent transgene expression important for the therapeutic effect, which were shown to be much more susceptible to stresses than titers.

Z-Averages around 25 nm were expected for unaggregated AAV2 formulations. DLS measurements revealed that addition of albumin resulted in low Z-Averages, indicating that no aggregation was prevented. Meanwhile all other AAV2 formulations revealed aggregation already at TO. Polydispersity measurements endorsed these conclusions and showed that formulation 6 was monodisperse. It was assumed that albumin binds to the capsid and prevented the occurrence of capsid-capsid interactions. Although freeze-drying has not significantly increased the Z-Average, formulations with albumin became polydisperse. Big Z-Average differences were seen between the formulations. Formulations 2, 3 and 7 had much smaller Z-Averages compared to formulations 1, 4, 5 and 8. With the current knowledge it is not possible to draw any conclusions if these formulations had favorable characteristics concerning vector aggregation. It can only be concluded that all formulations except formulation 6 aggregated.

Concentration problems were present in sub visible particle measurements with light obscuration. This method used volumes of 800-1000 μL. As described in the introduction, the formulation volume amounted to only 245 μL. Thus, samples were diluted to a titer of 1·10¹⁰ vg/mL. While sample dilution certainly can impair data quality and is sometimes not good practice, it was the only way to conduct such measurement. It was shown that particle counts in formulation vials are not bigger than in placebo vials. Particle counts did not exceed 1000 particles/mL. Higher particle counts in formulation and placebo 1 might be caused by auto-oxidation and hydrolysis of polysorbate 80. Method evaluation of zeta potential measurements showed that such measurements could be applied for AAV2 characterization. Surface potential measured during method evaluation correlated with the values described in the literature. Especially the formulation containing sodium hyaluronate revealed fluctuations within the zeta potential. Despite the fluctuations sodium hyaluronate caused the lowest zeta potential at TO. The binding of the negatively charged hyaluronic acid to the capsid may explain this finding. Zeta potential is an indicator for colloidal stability. The more negative or positive a zeta potential is, the higher is the repulsion between the particles and the smaller is their aggregation behavior. According to the literature all tested formulations have zeta potentials that reveal an incipient instability and that tend to aggregate [97]. Even though formulation 6 showed no aggregation, no significant changes in zeta potential could be observed. Results showed that only small capsid potential close to zero could be obtained with AAV2, indicating that capsids do not reveal big differences in potentials. Comparing the values with literature values of unaggregated AAV2 vectors it becomes noticeable that the surface potential is not impacted and therefore does not reliably reveal the colloidal stability of such vectors. This results questioned the use and benefits of zeta potential measurements in formulation development of AAV2. Zeta potential is influenced by the pH, which can be seen in formulation 1 revealing the biggest zeta potential. Acidic environment of the low pH formulation protonated the capsid with a pI of ˜6.4 leading to an increase in the surface potential.

It is worth mentioning that the three month time point was not fully analyzed, since the laboratory facilities were closed during the global COVID-19 breakout. Based on the previously obtained results, the implementation of the GFP expression assay was prioritized after three months of stability, and the data was shown and discusses above. Unfortunately, no time was left to conduct titer analysis as well as particle and zeta potential measurements.

### 7 Conclusion

In the first part of this Project analytical methods used for characterization of AAV2 were evaluated and developed. First, universal ITR qPCR was successfully optimized by exchanging the TaqMan approach with a SYBR-Green approach. The addition of 2 units of DNase I as well as implementation of a thermal capsid opening step improved the accuracy of the method. For monitoring transgene expression a plate reader assay was developed and it was shown that U2OS cells are a well suited cell line for quantifying AAV2 induced expression. Using TEM, it was shown that around 60% of the capsids were contained a viral DNA. Even though full and empty capsid ratio was successfully determined, measurements highlighted the need for a less time consuming and scalable approach. Using particle analysis with DLS, it was shown that a titer of 1·10¹¹ vg/ml could be detected but is at the lower limit of detection. Zeta potential measurements confirmed the literature values of −9.46 mV.

In a second step the analytical methods were applied and developed in a three month AAV2 formulation stability study. The first formulation study was terminated after a large titer loss was observed after compounding. These investigations showed that AAV2 aggregated quickly during compounding and accordingly were filtered out. Addition of 150 mM NaCl prevented aggregation and enabled sterile filtration. Only sterile filtration combined with a qPCR titration reliably showed that 150 mM NaCl significantly reduced aggregation.

In the second formulation study it was shown that addition of 1% albumin prevented aggregation during the course of this study. Whereas all other formulations aggregated already at T0. Formulation 6 and 8 showed no expression declines upon freezing and freeze-thaw cycles and are therefore suited as storage buffers. Transgene expression rapidly decreased upon storage in a temperature dependent way (40° C.>25° C.>2-8° C.≥−20/−80° C.). Freeze-drying followed by storage at 25° C. resulted in lower expressions compared to the respective liquid formulations stored at 25° C. This study showed that storage at 2-8° C. reached the expression minimum already after one month of storage. This illustrates the need of adding more pull points early in the study.

Titers stayed constant during the course of this study and were only affected by agitation stress. Addition of polysorbate 80 protected the formulations from a significant titer decline upon shaking, meanwhile P188 was not able to reveal a protective effect. It is demonstrated herein that supplementation of albumin and hyaluronic acid potentially interfered with qPCR titer quantifications.

With this evaluation and development of analytical methods and application of those within the formulation study, valuable insights in the field of AAV2 vector formulation development were gained.

### 8 References

[1] Wang, D., Tai, P. W. L. & Gao, G. Adeno-associated virus vector as a platform for gene therapy delivery. 18, 358-378 (2019).
[2] Anguela, X. M. & High, K. A. Entering the Modern Era of Gene Therapy. Annu. Rev. Med. 70, 273-288 (2019).
[3] Dunbar, C. et al. Human gene therapy comes of age. Science (80-.). 359, (2018).
[4] Rodrigues, G. A. et al. Pharmaceutical Development of AAV-Based Gene Therapy Products for the Eye. Pharm. Res. 36, (2019).
[5] Pettitt, D. et al. Emerging Platform Bioprocesses for Viral Vectors and Gene Therapies. Bioprocess Int. 14, (2016).
[6] Wells, D. J. Gene therapy progress and prospects: Electroporation and other physical methods. Gene Ther. 11, 1363-1369 (2004).
[7] Yin, H. et al. Non-viral vectors for gene-based therapy. Nat. Rev. Genet. 15, 541-555 (2014).
[8] Thomas, C. E., Ehrhardt, A. & Kay, M. A. Progress and problems with the use of viral vectors for gene therapy. Nat. Rev. Genet. 4, 346-358 (2003).
[9] Maetzig, T., Galla, M., Baum, C. & Schambach, A. Gammaretroviral vectors: Biology, technology and application. Viruses 3, 677-713 (2011).
[10] Robbins, P. D. & Ghivizzani, S. C. Viral vectors for gene therapy. Pharmacol. Ther. 80, 35-47 (1998).
[11] Lundstrom, K. Viral vectors in gene therapy. Diseases 6, (2017).
[12] Keeler, A. M., ElMallah, M. K. & Flotte, T. R. Gene Therapy 2017: Progress and Future Directions. Clin. Transl. Sci. 10, 242-248 (2017).
[13] Ginn, S. L., Amaya, A. K., Alexander, I. E., Edelstein, M. & Abedi, M. R. Gene therapy clinical trials worldwide to 2017: An update. J. Gene Med. 20, (2018).
[14] Buchschacher, G. L. & Wong-Staal, F. Development of lentiviral vectors for gene therapy for human diseases. Blood 95, 2499-2504 (2000).
[15] Vigna, E. & Naldini, L. Lentiviral vectors: excellent tools for experimental gene transfer and promising candidates for gene therapy. J. Gene Med. 2, 308-316 (2000).
[16] Manservigi, R., Argnani, R. & Marconi, P. HSV Recombinant Vectors for Gene Therapy. Open Virol. 1 4, 123-156 (2010).
[17] Chira, S. et al. Progresses towards safe and efficient gene therapy vectors. Oncotarget 6, 30675-30703 (2015).
[18] Bessis, N., GarciaCozar, F. J. & Boissier, M. C. Immune responses to gene therapy vectors: Influence on vector function and effector mechanisms. Gene Ther. 11, 10-17 (2004).
[19] Hollon, T. Researchers and regulators reflect on first gene therapy death. Nat. Med. 6, 6 (2000).
[20] Hacein-Bey-Abina, S. et al. Insertional oncogenesis in 4 patients after retrovirus-mediated gene therapy of SCID-X1 Find the latest version : Insertional oncogenesis in 4 patients after retrovirus-mediated gene therapy of SCID-X1. J Clin Invest 118, 3132-3142 (2008).
[21] Vance, M. A., Mitchell, A. & Samulski, R. J. AAV Biology, Infectivity and Therapeutic Use from Bench to Clinic. Gene Therapy—Principles and Challenges (2015).
[22] Scott, L. J. Alipogene tiparvovec: A review of its use in adults with familial lipoprotein lipase deficiency. Adis Drug Eval. 75, 175-182 (2015).
[23] De Giorgi, M. & Lagor, W. R. Gene Delivery in Lipid Research and Therapies. Methodist Debakey Cardiovasc. J. 15, 62-69 (2019).
[24] Trapani, I. & Auricchio, A. Seeing the Light after 25 Years of Retinal Gene Therapy. Trends Mol. Med. 24, 669-681 (2018).
[25] Al-Zaidy, S. A. & Mendell, J. R. From Clinical Trials to Clinical Practice: Practical Considerations for Gene Replacement Therapy in SMA Type 1. Pediatr. Neurol. 100, 3-11 (2019).
[26] Keeler, A. M. & Flotte, T. R. Recombinant Adeno-Associated Virus Gene Therapy in Light of Luxturna (and Zolgensma and Glybera): Where Are We, and How Did We Get Here? Annu. Rev. Virol. 6, 601-621 (2019).
[27] Croyle, M. A., Cheng, X. & Wilson, J. M. Development of formulations that enhance physical stability of viral vectors for gene therapy. Nat. Gene Ther. 8, 1281-1290 (2001).
[28] Xu, R. et al. Stability of infectious recombinant adeno-associated viral vector in gene delivery. Med. Sci. Monit. 11, 305-308 (2005).
[29] Gruntman, A. M. et al. Stability and compatibility of recombinant adeno-associated virus under conditions commonly encountered in human gene therapy trials. Hum. Gene Ther. Methods 26, 71-76 (2015).
[30] Wright, J. F. et al. Identification of factors that contribute to recombinant AAV2 particle aggregation and methods to prevent its occurrence during vector purification and formulation. Mol. Ther. 12, 171-178 (2005).
[31] Walters, R. W. et al. Binding of Adeno-associated Virus Type 5 to 2,3-Linked Sialic Acid is Required for Gene Transfer. J. Biol. Chem. 276, 20610-20616 (2001).
[32] Rayaprolu, V. et al. Comparative Analysis of Adeno-Associated Virus Capsid Stability and Dynamics. J. Virol. 87, 13150-13160 (2013).
[33] Venkatakrishnan, B. et al. Structure and Dynamics of Adeno-Associated Virus Serotype 1 VP1-Unique N-Terminal Domain and Its Role in Capsid Trafficking. J. Virol. 87, 4974-4984 (2013).
[34] Wang, Q. et al. High-Density Recombinant Adeno-Associated Viral Particles are Competent Vectors for in Vivo Transduction. Hum. Gene Ther. 27, 971-981 (2016).
[35] Johnson, J. S. et al. Mutagenesis of Adeno-Associated Virus Type 2 Capsid Protein VP1 Uncovers New Roles for Basic Amino Acids in Trafficking and Cell-Specific Transduction. J. Virol. 84, 8888-8902 (2010).
[36] Kronenberg, S., Bottcher, B., von der Lieth, C. W., Bleker, S. & Kleinschmidt, J. A. A Conformational Change in the Adeno-Associated Virus Type 2 Capsid Leads to the Exposure of Hidden VP1 N Termini. J. Virol. 79, 5296-5303 (2005).
[37] Bleker, S., Sonntag, F. & Kleinschmidt, J. A. Mutational Analysis of Narrow Pores at the Fivefold Symmetry Axes of Adeno-Associated Virus Type 2 Capsids Reveals a Dual Role in Genome Packaging and Activation of Phospholipase A2 Activity. J. Virol. 79, 2528-2540 (2005).
[38] Levy, H. C. et al. Heparin binding induces conformational changes in Adeno-associated virus serotype 2. J. Struct. Biol. 165, 146-156 (2009).
[39] Bartlett, J. S., Wilcher, R. & Samulski, R. J. Infectious entry pathway of adeno-associated virus and adeno-associated virus vectors. J. Virol. 74, 2777-2785 (2000).
[40] O'Donnell, J., Taylor, K. A. & Chapman, M. S. Adeno-associated virus-2 and its primary cellular receptor-Cryo-EM structure of a heparin complex. Virology 385, 434-443 (2009).
[41] Qing, K. et al. Human fibroblast growth factor receptor 1 is a co-receptor for infection by adeno-associated virus 2. Nat. Med. 5, 71-77 (1999).
[42] Pillay, S. et al. An essential receptor for adeno-associated virus infection. Nature 530, 108-112 (2016).
[43] Horowitz, E. D. et al. Biophysical and Ultrastructural Characterization of Adeno-Associated Virus Capsid Uncoating and Genome Release. J. Virol. 87, 2994-3002 (2013).
[44] Eichhoff, A. M. et al. Nanobody-Enhanced Targeting of AAV Gene Therapy Vectors. Mol. Ther.—Methods Clin. Dev. 15, 211-220 (2019).
[45] Samulski, R. J. & Muzyczka, N. AAV-Mediated Gene Therapy for Research and Therapeutic Purposes. Annu. Rev. Virol. 1, 427-51 (2014).
[46] Snyder, R. O. & Moullier, P. Adeno-Associated Virus. (Springer Protocols, 2011).
[47] Coura, R. D. S. & Nardi, N. B. The state of the art of adeno-associated virus-based vectors in gene therapy. Virol. J. 4, (2007).
[48] Wang Y, L. C., Wang L, L. Y. & Aslanidi GV, J. G. The Adeno-Associated Virus Genome Packaging Puzzle. J. Mol. Genet. Med. 09, (2015).
[49] McCarty, D. M. Self-complementary AAV vectors; advances and applications. Mol. Ther. 16, 1648-1656 (2008).
[50] Salganik, M., Hirsch, M. L. & Samulski, R. J. Adeno-associated Virus as a Mammalian DNA Vector. Microbiol Spectr 3, (2015).
[51] Aponte-Ubillus, J. J. et al. Molecular design for recombinant adeno-associated virus (rAAV) vector production. Appl. Microbiol. Biotechnol. 102, 1045-1054 (2018).
[52] Penaud-Budloo, M. et al. Adeno-Associated Virus Vector Genomes Persist as Episomal Chromatin in Primate Muscle. J. Virol. 82, 7875-7885 (2008).
[53] Schnepp, B. C., Clark, K. R., Klemanski, D. L., Pacak, C. A. & Johnson, P. R. Genetic Fate of Recombinant Adeno-Associated Virus Vector Genomes in Muscle. J. Virol. 77, 3495-3504 (2003).
[54] Dong, B., Nakai, H. & Xiao, W. Characterization of genome integrity for oversized recombinant AAV vector. Mol. Ther. 18, 87-92 (2010).
[55] Dorange, F. & Le Bec, C. Analytical approaches to characterize AAV vector production & purification: Advances and challenges. Cell Gene Ther. Insights 4, 119-129 (2018).
[56] Fagone, P. et al. Systemic errors in quantitative polymerase chain reaction titration of self-complementary adeno-associated viral vectors and improved alternative methods. Hum. Gene Ther. Methods 23, 1-7 (2012).
[57] Werling, N. J., Satkunanathan, S., Thorpe, R. & Zhao, Y. Systematic Comparison and Validation of Quantitative Real-Time PCR Methods for the Quantitation of Adeno-Associated Viral Products. Hum. Gene Ther. Methods 26, 82-92 (2015).
[58] Sommer, J. M. et al. Quantification of adeno-associated virus particles and empty capsids by optical density measurement. Mol. Ther. 7, 122-128 (2003).
[59] Arya, M. et al. Basic principles of real-time quantitative PCR. Expert Rev. Mol. Diagn. 5, 209-219 (2005).
[60] Ponchel, F. et al. Real-time PCR based on SYBR-Green I fluorescence: An alternative to the TaqMan assay for a relative quantification of gene rearrangements, gene amplifications and micro gene deletions. BMC Biotechnol. 3, 1-13 (2003).
[61] Tajadini, M., Panjehpour, M. & Javanmard, S. Comparison of SYBR Green and TaqMan methods in quantitative real-time polymerase chain reaction analysis of four adenosine receptor subtypes. Adv. Biomed. Res. 3, 85 (2014).
[62] Arikawa, E. et al. Cross-platform comparison of SYBR® Green real-time PCR with TaqMan PCR, microarrays and other gene expression measurement technologies evaluated in the MicroArray Quality Control (MAQC) study. BMC Genomics 9, 1-12 (2008).
[63] Zhang, B. et al. An evaluation of quantitative PCR assays (TaqMan® and SYBR Green) for the detection of Babesia bigemina and Babesia bovis, and a Novel fluorescent-ITS1-PCR capillary electrophoresis method for genotyping B. bovis isolates. Vet. Sci. 3, 1-15 (2016).
[64] Wang, F., Cui, X., Wang, M., Xiao, W. & Xu, R. A reliable and feasible qPCR strategy for titrating AAV vectors. Med. Sci. Monit. Basic Res. 19, 187-193 (2013).
[65] Aurnhammer, C. et al. Universal real-time PCR for the detection and quantification of adeno-associated virus serotype 2-derived inverted terminal repeat sequences. Hum. Gene Ther. Methods 23, 18-28 (2012).
[66] Rohr, U. P. et al. Fast and reliable titration of recombinant adeno-associated virus type-2 using quantitative real-time PCR. J. Virol. Methods 106, 81-88 (2002).
[67] D'Costa, S. et al. Practical utilization of recombinant AAV vector reference standards: focus on vector genomes titration by free ITR qPCR. Mol. Ther.—Methods Clin. Dev. 3, 16019 (2016).
[68] Furuta-Hanawa, B., Yamaguchi, T. & Uchida, E. Two-Dimensional Droplet Digital PCR as a Tool for Titration and Integrity Evaluation of Recombinant Adeno-Associated Viral Vectors. Hum. Gene Ther. Methods 30, 127-136 (2019).
[69] Hindson, B. J. et al. High-throughput droplet digital PCR system for absolute quantitation of DNA copy number. Anal. Chem. 83, 8604-8610 (2011).
[70] Dobnik, D. et al. Accurate Quantification and Characterization of Adeno-Associated Viral Vectors. Front. Microbiol. 10, (2019).
[71] Qu, G. et al. Separation of adeno-associated virus type 2 empty particles from genome containing vectors by anion-exchange column chromatography. J. Virol. Methods 140, 183-192 (2007).
[72] Xinghua, P. & Nazmul, K. Empty Capsids in Production of Viral Vector: Separation and Reassembly. J. Bioeng. Biomed. Sci. 8, (2018).
[73] Fu, X. et al. Analytical strategies for quantification of adeno-associated virus (AAV) empty capsids to support process development. Hum. Gene Ther. Methods 30, 144-152 (2019).
[74] Grieger, J. C., Soltys, S. M. & Samulski, R. J. Production of recombinant adeno-associated virus vectors using suspension HEK293 cells and continuous harvest of vector from the culture media for GMP FIX and FLT1 clinical vector. Mol. Ther. 24, 287-297 (2016).
[75] Lock, M., Alvira, M. R. & Wilson, J. M. Analysis of particle content of recombinant adeno-associated virus serotype 8 vectors by ion-exchange chromatography. Hum. Gene Ther. Methods 23, 56-64 (2012).
[76] Wang, C. et al. Developing an Anion Exchange Chromatography Assay for Determining Empty and Full Capsid Contents in AAV6.2. Mol. Ther.—Methods Clin. Dev. 15, 257-263 (2019).
[77] Nass, S. A. et al. Universal Method for the Purification of Recombinant AAV Vectors of Differing Serotypes. Mol. Ther.—Methods Clin. Dev. 9, 33-46 (2018).
[78] Burnham, B. et al. Analytical Ultracentrifugation as an Approach to Characterize Recombinant Adeno-Associated Viral Vectors. Hum. Gene Ther. Methods 26, 228-242 (2015).
[79] Grimm, D. et al. Titration of AAV-2 particles via a novel capsid ELISA: Packaging of genomes can limit production of recombinant AAV-2. Gene Ther. 6, 1322-1330 (1999).
[80] Kuck, D., Kern, A. & Kleinschmidt, J. A. Development of AAV serotype-specific ELISAs using novel monoclonal antibodies. J. Virol. Methods 140, 17-24 (2007).
[81] Kondratov, O. et al. Direct Head-to-Head Evaluation of Recombinant Adeno-associated Viral Vectors Manufactured in Human versus Insect Cells. Mol. Ther. 25, 2661-2675 (2017).
[82] Di Pasquale, G. et al. Identification of PDGFR as a receptor for AAV-5 transduction. Nat. Med. 9, 1306-1312 (2003).
[83] Duong, T. T. et al. Comparative AAV-EGFP transgene expression using vector serotypes 1-9, 7M8, and 8b in human pluripotent stem cells, RPEs, and human and rat cortical neurons. Stem Cells Int. (2019).
[84] Ellis, B. L. et al. A survey of ex vivo/in vitro transduction efficiency of mammalian primary cells and cell lines with Nine natural adeno-associated virus (AAV1-9) and one engineered adeno-associated virus serotype. Virol. J. 10, 1 (2013).
[85] Penaud-Budloo, M., François, A., Clement, N. & Ayuso, E. Pharmacology of Recombinant Adeno-associated Virus Production. Mol. Ther.—Methods Clin. Dev. 8, 166-180 (2018).
[86] Dey, S., Laredj, L., Damjanovic, K., Muller, M. & Beard, P. Growth of osteosarcoma cells in a three-dimensional bone-like matrix alters their susceptibility to adeno-associated virus. J. Gen. Virol. 95, 1539-1543 (2014).
[87] Nunez, R., Ackermann, M., Saeki, Y., Chiocca, A. & Fraefel, C. Flow cytometric assessment of transduction efficiency and cytotoxicity of herpes simplex virus type 1-based amplicon vectors. Cytometry 44, 93-99 (2001).
[88] Lorber, B., Fischer, F., Bailly, M., Roy, H. & Kern, D. Protein analysis by dynamic light scattering: Methods and techniques for students. Biochem. Mol. Biol. Educ. 40, 372-382 (2012).
[89] Stetefeld, J., McKenna, S. A. & Patel, T. R. Dynamic light scattering: a practical guide and applications in biomedical sciences. Biophys. Rev. 8, 409-427 (2016).
[90] Nikolai, N. et al. Comparative study of non-enveloped icosahedral viruses size. PLoS One 10, 1-11 (2015).
[91] Wright, J. F. Product-related impurities in clinical-grade recombinant AAV vectors: Characterization and risk assessment. Biomedicines 2, 80-97 (2014).
[92] Naso, M. F., Tomkowicz, B., Perry, W. L. & Strohl, W. R. Adeno-Associated Virus (AAV) as a Vector for Gene Therapy. BioDrugs 31, 317-334 (2017).
[93] Stieh, D. J. et al. Aggregate complexes of HIV-1 induced by multimeric antibodies. Retrovirology 11, 1-16 (2014).
[94] Vogel, R. et al. High-Resolution Single Particle Zeta Potential Characterisation of Biological Nanoparticles using Tunable Resistive Pulse Sensing. Sci. Rep. 7, 1-13 (2017).
[95] Bhattacharjee, S. DLS and zeta potential - What they are and what they are not? J. Control. Release 235, 337-351 (2016).
[96] Shao, W. et al. A novel polyethyleneimine-coated adenoassociated virus-like particle formulation for efficient siRNA delivery in breast cancer therapy: Preparation and in vitro analysis. Int. J. Nanomedicine 7, 1575-1586 (2012).
[97] Kumar, A. & Kumar Dixit, C. Methods for characterization of nanoparticles. in Advances in Nanomedicine for Delivery of Therapeutic Nucleic Acids 43-58 (2017).
[98] Wen, J., Lord, H., Knutson, N. & Wikström, M. Nano differential scanning fluorimetry for comparability studies of therapeutic proteins. Anal. Biochem. 593, 113581 (2020).
[99] Niesen, F. H., Berglund, H. & Vedadi, M. The use of differential scanning fluorimetry to detect ligand interactions that promote protein stability. Nat. Protoc. 2, 2212-2221 (2007).
[100] Ablinger, E., Leitgeb, S. & Zimmer, A. Differential scanning fluorescence approach using a fluorescent molecular rotor to detect thermostability of proteins in surfactant-containing formulations. Int. J. Pharm. 441, 255-260 (2013).
[101] Rieser, R. et al. Intrinsic Differential Scanning Fluorimetry for Fast and Easy Identification of Adeno-Associated Virus Serotypes. J. Pharm. Sci. 109, 854-862 (2020).
[102] Pacouret, S. et al. AAV-ID: A Rapid and Robust Assay for Batch-to-Batch Consistency Evaluation of AAV Preparations. Mol. Ther. 25, 1375-1386 (2017).
[103] Bennett, A. et al. Thermal Stability as a Determinant of AAV Serotype Identity. Mol. Ther.—Methods Clin. Dev. 6, 171-182 (2017).
[104] Wright, J. F., Qu, G., Tang, C. & Sommer, J. M. Recombinant adeno-associated virus: Formulation challenges and strategies for a gene therapy vector. Curr. Opin. Drug Discov. Dev. 6, 174-178 (2003).
[105] Wang, M. et al. Direct Interaction of Human Serum Proteins with AAV Virions to Enhance AAV Transduction: Immediate Impact on Clinical Applications. Gene Ther. 24, 49-59 (2017).

### 9 Appendix

TABLE 7
DLS back-scatter measurements during method evaluation

| Name | Mean | STD |
|---|---|---|
| Z-Average | 643 nm | 125 nm |
| PDI | 0.681 | 0.156 |
| Peak 1 | 26.7 nm | 2.87 |
| Peak 2 | 645 nm | 36.10 nm |

| | Back- | | Forward- |
|---|---|---|---|
| Name | scatter | Side-scatter | scatter |
| Peak 1 | 26.7 nm | 31 nm | 500 nm |
| Peak 2 | 643 nm | 1070 nm | — |

TABLE 8
Summary of all seeding densities, passages and cell viabilities for
used in the implementation of the GFP expression assay in
formulation study 2

| | Cell Stock | Cell | |
|---|---|---|---|
| | Suspension | viability | |
| Pull points | (cells/mL) | (%) | Passage |
| T0 | 1.05 · 10⁵ cells/mL | 99.3% | P4 |
| Five days, Agitation, | 1.05 · 10⁵ cells/mL | 100% | P7 |
| 200 rpm, 2-8° C. | | | |
| Two weeks, 25° C. | 1.05 · 10⁵ cells/mL | 100% | P7 |
| Two weeks, 40° C. | 1.05 · 10⁵ cells/mL | 100% | P7 |
| Freeze-Thaw, −20° C. | 1.07 · 10⁵ cells/mL | 99.8% | P10 |
| Freeze-Thaw, −80° C. | 1.07 · 10⁵ cells/mL | 99.8% | P10 |
| One month, 2-8° C. | 1.01 · 10⁵ cells/mL | 100% | P13 |
| One month, 25° C. | 1.01 · 10⁵ cells/mL | 100% | P13 |
| Three months, −80° C., | 1.07 · 10⁵ cells/mL | 100% | P5 |
| −20° C., 2-8° C., 25° C., | | | |
| Lyo 2-8° C., Lyo 25° C. | | | |

TABLE 9
Summary of all MOI used during the formulation study 2

| | MOI | | | | |
|---|---|---|---|---|---|
| | | T2W | T1M | T1M | T2W |
| | T0 | 25° C. | 2-8° C. | 25° C. | 40° C. |
| F1 | 5.32E+05 | 6.00E+05 | 1.15E+05 | 5.84E+05 | 9.37E+05 |
| F2 | 7.03E+05 | 7.91E+05 | 1.34E+05 | 5.89E+05 | 1.03E+06 |
| F3 | 2.00E+05 | 2.64E+05 | 8.90E+04 | 9.50E+04 | 3.45E+05 |
| F4 | 5.70E+05 | 9.61E+05 | 2.09E+05 | 5.84E+05 | 9.48E+05 |
| F5 | 6.07E+05 | 8.27E+05 | 1.14E+05 | 2.68E+05 | 4.16E+05 |
| F6 | 6.33E+04 | 4.86E+04 | 1.36E+05 | 9.19E+04 | 1.56E+04 |
| F7 | 7.75E+04 | 5.88E+05 | 2.04E+05 | 8.69E+04 | 6.32E+04 |
| F8 | 3.25E+05 | 5.20E+05 | 4.05E+04 | 2.77E+05 | 3.65E+05 |
| | MOI | | | | |
| | T1M Lyo | T1M | F/T | F/T | 5d, |
| | 2-8° C. | Lyo 25° C. | −80° C. | −20° C. | 200 rpm |
| F1 | — | — | 6.12E+05 | 5.07E+05 | 1.45E+05 |
| F2 | — | — | 9.99E+05 | 7.83E+05 | 5.49E+05 |
| F3 | 4.97E+05 | 1.41E+05 | 4.34E+05 | 5.07E+05 | 2.71E+04 |
| F4 | 3.95E+05 | 5.42E+05 | 7.49E+05 | 6.01E+05 | 2.38E+05 |
| F5 | — | — | 7.07E+05 | 6.75E+05 | 1.23E+05 |
| F6 | 1.41E+04 | 7.14E+04 | 2.03E+02 | 2.10E+04 | 2.60E+04 |
| F7 | 3.15E+04 | 3.43E+04 | 7.06E+04 | 5.88E+04 | 6.43E+04 |
| F8 | — | — | 4.10E+05 | 1.96E+05 | 2.22E+04 |

TABLE 10
GFP expression of formulations stored at 2-8° C.

| Pull points | F1 | STD | F2 | STD | F3 | STD | F4 | STD |
|---|---|---|---|---|---|---|---|---|
| 12 weeks −80° C. | 1.4E+06 | 2.0E+05 | 3.3E+06 | 3.3E+05 | 1.1E+06 | 1.3E+05 | 3.3E+06 | 4.5E+05 |
| 12 weeks −20° C. | 1.3E+06 | 1.1E+05 | 3.7E+06 | 3.1E+05 | 1.2E+06 | 1.8E+05 | 3.2E+06 | 4.0E+05 |
| T0 | 3.7E+06 | 1.8E+05 | 5.4E+06 | 4.2E+05 | 1.4E+06 | 1.4E+05 | 5.8E+06 | 2.6E+05 |
| 4 weeks | 1.8E+06 | 4.0E+05 | 3.5E+06 | 1.8E+05 | 4.1E+05 | 1.2E+05 | 3.4E+06 | 1.8E+05 |
| 12 weeks | 1.1E+06 | 7.5E+04 | 3.2E+06 | 1.2E+05 | 4.3E+05 | 1.0E+05 | 3.3E+06 | 9.9E+04 |
| 4 weeks lyo | — | — | — | — | 5.1E+05 | 1.1E+05 | 3.0E+06 | 2.7E+05 |
| 12 weeks lyo | — | — | — | — | 6.1E+05 | 1.1E+05 | 2.1E+06 | 5.2E+05 |
| Pull points | F5 | STD | F6 | STD | F7 | STD | F8 | STD |
| 12 weeks −80° C. | 3.7E+06 | 3.1E+05 | 4.7E+06 | 2.5E+05 | 4.3E+06 | 2.6E+05 | 4.8E+06 | 1.1E+05 |
| 12 weeks −20° C. | 4.2E+06 | 2.8E+05 | 5.0E+06 | 1.4E+05 | 4.3E+06 | 1.4E+05 | 4.5E+06 | 2.9E+05 |
| T0 | 5.7E+06 | 4.1E+05 | 5.6E+06 | 8.8E+05 | 5.0E+06 | 1.6E+05 | 4.5E+06 | 1.2E+05 |
| 4 weeks | 3.6E+06 | 3.4E+05 | 4.4E+06 | 3.8E+05 | 3.3E+06 | 1.7E+05 | 2.6E+06 | 1.7E+05 |
| 12 weeks | 3.8E+06 | 1.1E+05 | 4.6E+06 | 2.8E+05 | 3.5E+06 | 1.1E+05 | 3.1E+06 | 2.3E+05 |
| 4 weeks lyo | — | — | 3.7E+06 | 1.9E+05 | 1.8E+06 | 8.5E+04 | — | — |
| 12 weeks lyo | — | — | 3.2E+06 | 1.8E+05 | 2.3E+06 | 2.3E+05 | — | — |

TABLE 11
GFP expression of formulations stored at 25° C.

| Pull points | F1 | STD | F2 | STD | F3 | STD | F4 | STD |
|---|---|---|---|---|---|---|---|---|
| 12 weeks −80° C. | 1.4E+06 | 2.0E+05 | 3.3E+06 | 3.3E+05 | 1.1E+06 | 1.3E+05 | 3.3E+06 | 4.5E+05 |
| 12 weeks −20 | 1.3E+06 | 1.1E+05 | 3.7E+06 | 3.1E+05 | 1.2E+06 | 1.8E+05 | 3.2E+06 | 4.0E+05 |
| T0 | 3.7E+06 | 1.8E+05 | 5.4E+06 | 4.2E+05 | 1.4E+06 | 1.4E+05 | 5.8E+06 | 2.6E+05 |
| 2 weeks | 5.0E+05 | 1.7E+04 | 9.4E+05 | 1.1E+05 | 2.9E+05 | 5.6E+04 | 2.0E+06 | 2.7E+05 |
| 4 weeks | 1.7E+05 | 5.4E+04 | 9.5E+05 | 9.1E+04 | 2.9E+05 | 7.7E+04 | 2.3E+06 | 1.6E+05 |
| 12 weeks | 7.1E+04 | 1.2E+04 | 3.0E+05 | 2.2E+04 | 2.9E+05 | 5.4E+04 | 9.4E+05 | 1.1E+05 |
| 4 weeks lyo | — | — | — | — | 2.3E+05 | 5.9E+04 | 1.2E+06 | 1.4E+05 |
| 12 weeks lyo | — | — | — | — | 2.1E+05 | 4.4E+04 | 6.9E+05 | 5.6E+04 |
| Pull points | F5 | STD | F6 | STD | F7 | STD | F8 | STD |
| 12 weeks −80° C. | 3.7E+06 | 3.1E+05 | 4.7E+06 | 2.5E+05 | 4.3E+06 | 2.6E+05 | 4.8E+06 | 1.1E+05 |
| 12 weeks −20 | 4.2E+06 | 2.8E+05 | 5.0E+06 | 1.4E+05 | 4.3E+06 | 1.4E+05 | 4.5E+06 | 2.9E+05 |
| T0 | 5.7E+06 | 4.1E+05 | 5.6E+06 | 8.8E+05 | 5.0E+06 | 1.6E+05 | 4.5E+06 | 1.2E+05 |
| 2 weeks | 2.7E+06 | 3.3E+05 | 2.9E+06 | 1.1E+05 | 2.3E+06 | 1.9E+05 | 2.7E+06 | 2.5E+05 |
| 4 weeks | 2.9E+06 | 8.6E+04 | 2.9E+06 | 9.4E+04 | 2.2E+06 | 2.0E+05 | 2.2E+06 | 2.7E+05 |
| 12 weeks | 2.5E+06 | 3.5E+05 | 1.7E+06 | 6.6E+04 | 1.2E+06 | 3.9E+04 | 1.2E+06 | 9.3E+04 |
| 4 weeks lyo | — | — | 1.6E+06 | 1.1E+05 | 1.2E+06 | 1.5E+05 | — | — |
| 12 weeks lyo | — | — | 8.8E+05 | 1.1E+05 | 6.4E+05 | 2.4E+04 | — | — |

TABLE 12
GFP expression after exposure to different stresses

| Pull points | F1 | STD | F2 | STD | F3 | STD | F4 | STD |
|---|---|---|---|---|---|---|---|---|
| T0 | 3.7E+06 | 1.8E+05 | 5.4E+06 | 4.2E+05 | 1.4E+06 | 1.4E+05 | 5.8E+06 | 2.6E+05 |
| F/T −80° C. | 2.0E+06 | 2.3E+05 | 4.2E+06 | 3.5E+05 | 1.3E+06 | 1.3E+05 | 4.8E+06 | 3.6E+05 |
| F/T −20° C. | 1.7E+06 | 1.9E+05 | 3.7E+06 | 3.4E+05 | 7.2E+05 | 5.5E+04 | 3.2E+06 | 3.3E+05 |
| 5d, 200 rpm | 1.4E+06 | 1.9E+05 | 3.4E+06 | 3.4E+05 | 1.7E+05 | 5.5E+04 | 2.3E+06 | 3.3E+05 |
| 2 weeks 40° C. | −2.6E+03 | 5.1E+03 | 5.2E+03 | 1.0E+04 | 2.4E+04 | 9.0E+03 | 9.3E+04 | 1.2E+04 |
| Pull points | F5 | STD | F6 | STD | F7 | STD | F8 | STD |
| T0 | 5.7E+06 | 4.1E+05 | 5.6E+06 | 8.8E+05 | 5.0E+06 | 1.6E+05 | 4.5E+06 | 1.2E+05 |
| F/T −80° C. | 4.1E+06 | 3.6E+05 | 5.1E+06 | 3.8E+05 | 2.9E+06 | 2.6E+05 | 4.9E+06 | 2.2E+05 |
| F/T −20° C. | 2.5E+06 | 2.8E+05 | 4.8E+06 | 6.2E+05 | 2.7E+06 | 4.1E+05 | 4.5E+06 | 2.2E+05 |
| 5d, 200 rpm | 2.4E+06 | 2.8E+05 | 3.9E+06 | 6.2E+05 | 3.2E+06 | 4.1E+05 | 2.2E+06 | 2.2E+05 |
| 2 weeks 40° C. | 3.2E+05 | 4.0E+04 | 2.1E+05 | 1.9E+04 | 9.5E+04 | 2.4E+04 | 1.2E+05 | 1.6E+04 |

TABLE 13
Summary of all measured Z-Averages (in nm)

| | F1 | STD | F2 | STD | F3 | STD | F4 | STD | F5 | STD | F6 | STD | F7 | STD | F8 |
|---|---|---|---|---|---|---|---|---|---|---|---|---|---|---|---|
| TO | 1560 | 150 | 400 | 7 | 581 | 182 | 1135 | 21 | 1193 | 147 | 24 | 6 | 879 | 65 | 1170 |
| T2W 40° C. | 2046 | 189 | 405 | 17 | 982 | 67 | 1378 | 61 | 1590 | 99 | 31 | 5 | 977 | 89 | 1481 |
| T2W 25° C. | 1636 | 190 | 636 | 98 | 477 | 187 | 2367 | 113 | 1970 | 103 | 13 | 2 | 2121 | 245 | 2493 |
| TIM 2- | 1713 | 151 | 1085 | 28 | 217 | 59 | 1609 | 132 | 1146 | 186 | 17 | 2 | 371 | 39 | 1821 |
| 8° C. | | | | | | | | | | | | | | | |
| TIM 25° C. | 4042 | 304 | 255 | 28 | 886 | 170 | 3465 | 112 | 2807 | 500 | 11 | 2 | 2374 | 509 | 2423 |
| F/T −80° C. | 892 | 17 | 732 | 68 | 864 | 171 | 1250 | 223 | 1150 | 77 | 26 | 6 | 356 | 27 | 1792 |
| F/T−20° C. | 545 | 15 | 758 | 15 | 510 | 30 | 1240 | 57 | 840 | 101 | 29 | 5 | 439 | 87 | 1134 |
| TIM Lyo | — | — | — | — | 401 | 45 | 367 | 25 | — | — | 23 | 4 | 382 | 84 | — |
| 2-8° C. | | | | | | | | | | | | | | | |
| TIM Lyo | — | — | — | — | 357 | 39 | 173 | 0 | — | — | 23 | 5 | 386 | 70 | — |
| 25° C. | | | | | | | | | | | | | | | |
| 5d, 200 | 961 | 98 | 550 | 66 | 553 | 11 | 652 | 101 | 667 | 211 | 9 | 0 | 401 | 126 | 1630 |
| rpm | | | | | | | | | | | | | | | |

TABLE 14
Summary of all polydispersity indexes

| | F1 | STD | F2 | STD | F3 | STD | F4 | STD | F5 | STD | F6 | STD | F7 | STD | F8 |
|---|---|---|---|---|---|---|---|---|---|---|---|---|---|---|---|
| TO | 0.79 | 0.02 | 0.72 | 0.07 | 0.63 | 0.00 | 0.85 | 0.01 | 0.27 | 0.08 | 0.30 | 0.03 | 0.93 | 0.13 | 0.72 |
| T2W 40° C. | 0.94 | 0.29 | 0.63 | 0.12 | 0.75 | 0.06 | 0.72 | 0.06 | 0.79 | 0.05 | 0.25 | 0.01 | 0.89 | 0.21 | 0.59 |
| T2W 25° C. | 0.90 | 0.01 | 0.56 | 0.17 | 0.52 | 0.04 | 1.04 | 0.10 | 0.95 | 0.11 | 0.26 | 0.02 | 0.78 | 0.05 | 0.39 |
| TIM 2-8° C. | 0.99 | 0.05 | 0.51 | 0.07 | 0.66 | 0.09 | 0.44 | 0.22 | 0.83 | 0.05 | 0.26 | 0.01 | 0.88 | 0.04 | 0.72 |
| TIM 25° C. | 1.00 | 0.11 | 0.64 | 0.07 | 0.72 | 0.08 | 1.23 | 0.06 | 0.74 | 0.18 | 0.23 | 0.01 | 0.80 | 0.08 | 1.01 |
| F/T −80° C. | 0.47 | 0.06 | 0.63 | 0.19 | 0.61 | 0.22 | 0.81 | 0.12 | 0.66 | 0.01 | 0.30 | 0.03 | 0.56 | 0.03 | 0.45 |
| F/T −20° C. | 0.60 | 0.05 | 0.53 | 0.03 | 0.71 | 0.05 | 0.57 | 0.05 | 0.53 | 0.19 | 0.33 | 0.02 | 1.29 | 0.24 | 0.59 |
| TIM Lyo 2- | — | — | — | — | 0.54 | 0.03 | 1.03 | 0.23 | — | — | 0.59 | 0.03 | 0.60 | 0.00 | — |
| 8° C. | | | | | | | | | | | | | | | |
| TIM Lyo | — | — | — | — | 0.53 | 0.04 | 1.43 | 0.02 | — | — | 0.52 | 0.06 | 0.66 | 0.07 | — |
| 25° C. | | | | | | | | | | | | | | | |
| 5d, 200 rpm | 0.78 | 0.05 | 0.75 | 0.13 | 0.67 | 0.01 | 0.78 | 0.18 | 0.87 | 0.08 | 0.23 | 0.03 | 0.69 | 0.05 | 1.05 |

TABLE 15
Back-scatter DLS results of all Placebos at T0

| | P1 | P2 | P3 | P4 | P5 | P6 | P7 | P8 |
|---|---|---|---|---|---|---|---|---|
| Z-Average | 4.39 | 8.11 | 7.51 | 4.17 | 3.18 | 8.54 | 3.67 | 3.24 |
| (nm) | | | | | | | | |
| Polydispersity | 0.40 | 0.69 | 0.64 | 0.39 | 0.31 | 0.15 | 0.36 | 0.32 |
| index | | | | | | | | |
| Peak 1 (nm) | 1.19 | 1.16 | 1.14 | 1.25 | 1.23 | 10.00 | 1.20 | 1.27 |
| Peak 2 (nm) | 20.30 | 12.60 | 12.50 | 26.30 | 21.50 | — | 72.60 | 285.00 |
| Peak 3 (nm) | 245.30 | 242.00 | 219.00 | — | — | — | — | — |

TABLE 16
Summary of all zeta potentials measured during formulation study 2

| | Zeta potential (mV) | | | | | | | | | |
|---|---|---|---|---|---|---|---|---|---|---|
| | | | | | T2W | | | | TIM | |
| | T0 | STD | T2W40C | STD | 25° C. | STD | TIM 2-8° C. | STD | 25° C. | STD |
| F1 | −5.5 | 0.8 | −7.0 | 0.6 | −5.8 | 0.6 | −7.0 | 1.1 | − 5.2 | 0.4 |
| F2 | −15.0 | 1.3 | −8.9 | 1.1 | −13.0 | 0.4 | −9.9 | 5.1 | −15.2 | 0.3 |
| F3 | −7.1 | 3.6 | −6.7 | 0.4 | −12.8 | 0.7 | −10.3 | 3.5 | −8.7 | 1.8 |
| F4 | −4.5 | 1.1 | −8.0 | 1.4 | −15.4 | 1.0 | −2.8 | 0.9 | −13.8 | 0.9 |
| F5 | −16.0 | 1.01 | −11.0 | 0.9 | −9.6 | 0.5 | −11.2 | 3.0 | −10.5 | 1.7 |
| F6 | −12.7 | 1.2 | −12.7 | 1.3 | −17.9 | 0.8 | −6.6 | 0.2 | −13.5 | 0.8 |
| F7 | −17.5 | 0.7 | −31.3 | 7.2 | −13.5 | 0.8 | −7.3 | 0.5 | −6.0 | 3.9 |
| F8 | −9.5 | 0.4 | −9.2 | 2.2 | −11.1 | 1.0 | −11.1 | 0.8 | −11.0 | 4.2 |
| | Zeta potential (mV) | | | | | | | | | |
| | F/T | STD | F/T | | 5d, 200 | | TIM Lyo | | T1M Lyo | |
| | −80° C. | | −20° C. | STD | rpm | STD | 2-8° C. | STD | 25° C. | STD |
| F1 | −0.1 | 0.2 | −2.4 | 1.4 | −5.3 | 0.7 | | | | |
| F2 | −7.3 | 0.9 | −4.3 | 2.0 | −11.9 | 0.5 | | | | |
| F3 | −7.0 | 0.9 | −5.9 | 1.0 | −4.6 | 5.3 | −9.1 | 0.8 | −11.1 | 1.0 |
| F4 | −12.9 | 1.2 | −9.0 | 0.3 | −8.5 | 2.3 | −3.1 | 0.6 | −10.1 | 0.8 |
| F5 | −13.2 | 0.7 | −14.5 | 0.9 | −14.1 | 6.6 | | | | |
| F6 | −11.8 | 0.7 | −15.9 | 1.7 | −4.5 | 1.8 | −11.5 | 1.4 | −13.8 | 0.7 |
| F7 | −15.0 | 2.1 | −2.2 | 0.8 | −14.4 | 1.5 | −23.0 | 2.5 | −11.4 | 5.0 |
| F8 | −10.3 | 0.6 | −6.3 | 2.9 | −4.5 | 4.6 | | | | |

TABLE 17
Summary of all pH measurements of formulation study 2 4855-2211-1860, v. 1

| | | T2W | T1M | T2W | T1M | F/T | F/T | TIM Lyo | T1M Lyo | 5d, 200 |
|---|---|---|---|---|---|---|---|---|---|---|
| | T0 | 40° C. | 2-8° C. | 25° C. | 25° C. | −80° C. | −20° C. | 2-8° C. | 25° C. | rpm |
| F1 | 5.528 | 5.568 | 5.853 | 5.876 | 5.614 | 5.626 | 5.785 | — | — | 5.527 |
| F2 | 6.810 | 6.820 | 6.783 | 6.758 | 6.833 | 6.838 | 6.979 | — | — | 6.818 |
| F3 | 6.744 | 6.803 | 6.845 | 6.764 | 6.784 | 6.782 | 6.741 | 6.770 | 6.769 | 6.783 |
| F4 | 6.702 | 6.723 | 6.706 | 6.711 | 6.723 | 6.732 | 6.746 | 6.716 | 6.705 | 6.713 |
| F5 | 7.390 | 7.393 | 7.388 | 7.390 | 7.401 | 7.411 | 7.422 | — | — | 7.388 |
| F6 | 6.645 | 6.656 | 6.638 | 6.640 | 6.658 | 6.705 | 6.716 | 6.663 | 6.670 | 6.655 |
| F7 | 6.688 | 6.718 | 6.714 | 6.720 | 6.715 | 6.743 | 6.751 | 6.713 | 6.725 | 6.717 |
| F8 | 6.691 | 6.715 | 6.713 | 6.713 | 6.718 | 6.748 | 6.722 | — | — | 6.732 |
| | | T2W | T1M | T2W | T1M | F/T | F/T | TIM Lyo | TIM Lyo | 5d, 200 |
| | T0 | 40° C. | 2-8° C. | 25° C. | 25° C. | 80° C. | −20° C. | 2-8° C. | 25° C. | rpm |
| P1 | 5.486 | 5.514 | 5.549 | 5.460 | 5.520 | 5.555 | 5.654 | — | — | 5.494 |
| P2 | 6.781 | 6.769 | 6.780 | 6.756 | 6.792 | 6.832 | 6.911 | — | — | 6.784 |
| P3 | 6.724 | 6.774 | 6.770 | 6.758 | 6.780 | 6.790 | 6.770 | 6.790 | 6.790 | 6.770 |
| P4 | 6.684 | 6.698 | 6.715 | 6.699 | 6.725 | 6.735 | 6.720 | 6.727 | 6.728 | 6.716 |
| P5 | 7.379 | 7.395 | 7.390 | 7.374 | 7.391 | 7.422 | 7.422 | — | — | 7.392 |
| P6 | 6.624 | 6.660 | 6.742 | 6.723 | 6.663 | 6.684 | 6.687 | 6.676 | 6.670 | 6.791 |
| P7 | 6.685 | 6.719 | 6.708 | 6.713 | 6.719 | 6.728 | 6.745 | 6.719 | 6.717 | 6.702 |
| P8 | 6.676 | 6.713 | 6.717 | 6.709 | 6.722 | 6.745 | 6.738 | — | — | 6.722 |

1-103. (canceled)

104. A formulation comprising:
a. a viral vector;
b. a buffer; and
c. a polysaccharide, wherein the polysaccharide is sodium hyalauronate.

105. The formulation of claim 104, wherein the formulation is a liquid, and wherein the formulation comprises about 0.01 ng/mL to about 1 mg/mL, about 0.05 ng/mL to about 0.5 mg/mL, about 0.1 ng/mL to about 0.3 mg/mL, about 0.15 ng/mL to about 0.25 mg/mL, or about 0.2 ng/mL sodium hyaluronate, and/or wherein the formulation comprises about 2 mM to about 100 mM, about 5 mM to about 50 mM, about 15 mM to about 25 mM, or about 20 mM buffer.

106. A method for reducing aggregation of a viral vector in a formulation, comprising formulating the viral vector in the formulation of claim 104.

107. The formulation of claim 104, wherein the formulation further comprises a globular protein, and wherein the globular protein is albumin, alpha-fetoprotein, vitamin D-binding protein, afamin, a globin protein, alpha globulin, beta globulin, gamma globulin, or combinations thereof

108. The formulation of claim 107, wherein the formulation comprises about 0.1% to about 5.0%, about 0.5% to about 2.0%, about 0.75% to about 1.5%, about 0.8% to about 1.2%, or about 1.0% globular protein.

109. The formulation of claim 104, wherein the formulation further comprises a sugar, and wherein the sugar is sucrose, lactose, glucose, trehalose, or combinations thereof

110. The formulation of claim 109, wherein the formulation is a liquid and comprises about 50 mM to about 500 mM, about 100 mM to about 400 mM, about 250 mM to about 350 mM, or about 290 mM sugar.

111. The formulation of claim 104, wherein the formulation further comprises a surfactant, and wherein the surfactant is polysorbate 80, polysorbate 20 or Kolliphor P188.

112. The formulation of claim 111, wherein the formulation comprises about 0.01% to about 0.1% or about 0.015% to about 0.025% surfactant.

113. The formulation of claim 104, wherein the formulation is a freeze-dried solid.

114. A method of preparing a viral vector formulation, the method comprising:
a. obtaining a composition comprising the viral vector;
b. adding sodium hyaluronate to the composition of (a) to form the formulation, wherein the sodium hyaluronate is added in an amount sufficient to provide a Z-average of less than or equal to about 50 nm or a polydispersity index of less than or equal to about 0.5.

115. A formulation comprising:
a. a viral vector;
b. a buffer;
c. a globular protein; and
d. a sugar,
wherein the sugar is sucrose, lactose, glucose, trehalose, or combinations thereof

116. The formulation of claim 115, wherein the formulation further comprises a polysaccharide, and wherein the polysaccharide is sodium hyaluronate, heparan, heparan sulfate, heparin, chondroitin, chondroitin sulfate, dermatan, dermatan sulfate, keratan, keratan sulfate, algin, chitosan, chitosan sulfate, dextran, dextran sulfate or combinations thereof

117. The formulation of claim 116, wherein the formulation is a liquid and comprises about 0.01 ng/mL to about 1 mg/mL, about 0.05 ng/mL to about 0.5 mg/mL, about 0.1 ng/mL to about 0.3 mg/mL, about 0.15 ng/mL to about 0.25 mg/mL, or about 0.2 ng/mL polysaccharide.

118. The formulation of claim 117, wherein the formulation comprises about 0.01% to about 0.1% or about 0.015% to about 0.025% surfactant.

119. The formulation of claim 115, wherein the formulation further comprises a surfactant, and wherein the surfactant is polysorbate 80, polysorbate 20 or Kolliphor P188.

120. The formulation of claim 115, wherein the globular protein is albumin, alpha-fetoprotein, vitamin D-binding protein, afamin, a globin protein, alpha globulin, beta globulin, gamma globulin, or combinations thereof

121. The formulation of claim 115, wherein the formulation is a liquid and comprises about 0.1% to about 5.0%, about 0.5% to about 2.0%, about 0.75% to about 1.5%, about 0.8% to about 1.2%, or about 1.0% globular protein, and/or wherein the formulation comprises about 2 mM to about 100 mM, about 5 mM to about 50 mM, about 15 mM to about 25 mM, or about 10 mM buffer, and/or wherein the formulation comprises about 50 mM to about 500 mM, about 100 mM to about 400 mM, about 250 mM to about 350 mM. or 290 mM sugar.

122. The formulation of claim 115, wherein the viral vector is an adeno-associated viral vector, an adenoviral vector, a lentiviral vector, a retroviral vector, a herpes simplex viral vector or a hybrid vector, and wherein the sugar is sucrose or trehalose.

123. The formulation of claim 115, wherein the buffer is sodium phosphate, L-histidine, tris, succinate, sodium citrate or combinations thereof.

124. A formulation comprising:
a. a viral vector;
b. a buffer;
c. polysorbate 80; and
d. a sugar,
wherein the sugar is sucrose, lactose, glucose, trehalose, or combinations thereof

125. The formulation of claim 124, wherein the formulation further comprises a polysaccharide, globular protein, and a surfactant.

126. A method of preparing a viral vector formulation, the method comprising:
a. obtaining a composition comprising the viral vector;
b. adding a globular protein to the composition of (a) to form the formulation,
wherein the globular protein is added in an amount sufficient to provide a Z-average of less than or equal to about 50 nm or to provide a polydispersity index of less than or equal to about 0.5.


SEQUENCE LISTING (2 sequences)

SEQ ID NO: 1 - DNA, 21 bp, Artificial Sequence
  Forward Primer
ggaacccctagtgatggagtt

SEQ ID NO: 2 - DNA, 16 bp, Artificial Sequence
  Reverse Primer
cggcctcagtgagcga